SEQUENCE LISTING (3 sequences)

SEQ ID NO: 1 - protein, 355 aa, Homo sapiens
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMBNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 2 - protein, 353 aa, mouse
MALFSAQSPYINPIIPFTGPIQGGLQEGLQVTLQGTTKSFAQRFVVNFQNSFNGNDIAFHFNPRFEEGGYVVCNTKQNGQWGPEERKMQMPFQKGMPEFELCFLVQRSEFKVMVNKKFVQYQHRVPYHLVDTIAVSGCLKLSFITFQNSAAPVQHVFSTVQFSQPVQFPRTPKGRKQKTQNFRPAHQAPMAQTTIHMVHSTPGQMFSTPGIPPVVYPTPAYTIPFYTPIPNGLYPSKSIMISGNVLPDATRFHINLRCGGDIAFHLNPRFNENAVVRNTQINNSWGQEERSLLGRMPFSRGQSFSVWIICEGHCFKVAVNGQHMCEYYHRLKNLQDINTLEVAGDIQLTHVQT

SEQ ID NO: 3 - protein, 247 aa, artificial sequence
  protein alignment
MAQPYPPFGIQGGLQGLQTGTSRFVNFQFGNDIAFHFNPRFEGGYVVCNTQNGWGPEERKMPFQKGMPFLCFLVQSFKVMVNFVQYHRVPHVDTIVGLSIFQNPVQFSTVFSQPVFPPGRQKPAAPQTIHVSPGQMFSTPIPPYPPAYPFTIGLYPSKSISGVLPARFHINLGIAFHLNPRFENAVVRNTQINSWGEERSLMPFRGQSFSVWICEHCKVAVGQHEYYHRLNLINLEVGDIQLTHVQT

(19) United States
(12) Patent Application Publication
(10) Pub. No.: US 2018/0044422 A1
(43) Pub. Date: Feb. 15, 2018

(54) TREATING SOLID TUMOR BY TARGETING DECTIN-1 SIGNALING

(71) Applicant: New York University, New York, NY (US)

(72) Inventors: George Miller, Englewood, NJ (US); Donnele Daley, New York, NY (US); Vishnu R. Mani, New York, NY (US)

(21) Appl. No.: 15/664,720

(22) Filed: Jul. 31, 2017

Related U.S. Application Data
(60) Provisional application No. 62/368,434, filed on Jul. 29, 2016.
(60) Provisional application No. 62/483,161, filed on Apr. 7, 2017.

(51) Int. Cl. C07K 16/28; G01N 33/574; A61K 45/06; A61K 31/713; A61K 39/395; C12N 15/113

(52) U.S. Cl. CPC C07K 16/2851; A61K 39/39558; C07K 16/2818; C12N 15/1138; A61K 45/06; A61K 31/713; G01N 33/57438; A61K 2039/507

(57) ABSTRACT

Compositions and methods for treating solid tumors such as pancreatic ductal adenocarcinoma (PDA) via interfering with the Dectin-1 signaling pathway, for example, using Dectin-1 or Galectin-9 inhibitors.

### CROSS-REFERENCE TO RELATED APPLICATIONS

This application claims priority to U.S. Provisional Patent Application No. 62/368,434, filed on Jul. 29, 2016, and U.S. Provisional Patent Application No. 62/483,161, filed on Apr. 7, 2017, the disclosures of which are incorporated herein by reference.

### FEDERALLY SPONSORED RESEARCH

This invention was made with government support under Grant Nos. CA-155649, CA-168611, and CA-193111, awarded by the National Institutes of Health. The Government has certain rights in the invention.

### BACKGROUND OF DISCLOSURE

Pancreatic ductal adenocarcinoma (PDA) is a devastating disease with few long-term survivors (Yadav et al., Gastroenterology, 2013, 144, 1252-1261). Inflammation is paramount in PDA progression as oncogenic mutations alone, in the absence of concomitant inflammation, are insufficient for tumorigenesis (Guerra et al., Cancer Cell, 2007, 11, 291-302). Innate and adaptive immunity cooperate to promote tumor progression in PDA. In particular, specific innate immune subsets within the tumor microenvironment (TME) are apt at educating adaptive immune effector cells towards a tumor-permissive phenotype. Antigen presenting cell (APC) populations, including M2-polarized tumor-associated macrophages (TAMs) and myeloid dendritic cells (DC), induce the generation of immune suppressive Th2 cells in favor of tumor-protective Th1 cells (Ochi et al., J of Exp Med., 2012, 209, 1671-1687; Zhu et al., Cancer Res., 2014, 74, 5057-5069). Similarly, it has been shown that myeloid derived suppressor cells (MDSC) negate anti-tumor CD8⁺ cytotoxic T-Lymphocyte (CTL) responses in PDA and promote metastatic progression (Connolly et al., J Leuk Biol., 2010, 87, 713-725; Pylayeva-Gupta et al., Cancer Cell, 2012, 21, 836-847; Bayne et al., Cancer Cell, 2012, 21, 822-835). However, the drivers of monocytic cellular differentiation toward an immune-suppressive phenotype remain uncertain.

Dectin-1 is a member of the C-type lectin family of pattern recognition receptors and is expressed on the surface of macrophages and other cells of the myeloid-monocytic lineage (Goodridge et al., Nature, 2011, 472, 471-475). Dectin-1 is a crucial component of the innate immune system's ability to recognize beta-glucan polysaccharides derived from fungal cell walls (Taylor et al., Nature Immunol., 2007, 8, 31-38). Ligation of Dectin-1 by β-glucans recruits the CARD9 adaptor protein, which phosphorylates Syk, thereby initiating an anti-fungal immune response (Strasser et al., Immunity, 2012, 36, 32-42; Gross et al., Nature, 2006, 442, 651-656). Recent work suggested that chronic inflammatory injury upregulates Dectin-1 expression in the liver (Seifert et al., Cell Reports, 2015, 13, 1909-1921). Further, it was found that the PDA TME is rife with damage associated molecular patterns (DAMPs) generated as byproducts of inflammation and necrotic cell death (Ochi et al., J Exp Med., 2012, 209, 1671-1687; Seifert et al., Nature, 2016, 532, 245-249). However, unlike Toll-like receptors, which influence oncogenic progression when ligated by DAMPs (Ochi et al., J Exp Med., 2012, 209, 1671-1687; Zambirinis et al., J Exp Med., 2015, 212, 2077-2094; Ochi et al., J Clin Invest., 2012, 122, 4118-4129), the role of Dectin-1 in non-pathogen mediated inflammation or oncogenesis is not well-defined and sterile Dectin-1 ligands have not been characterized.

### SUMMARY OF DISCLOSURE

The present disclosure is based, at least in part, on the findings that Dectin-1 is overly expressed in PDA TME and Dectin-1 ligation in macrophages drives their immune suppressive cellular differentiation in PDA and thereby governs the tolerogenic T cell program in the TME, which facilitates oncogenic progression. It was further found that Dectin-1 antagonists successfully blocked tumor growth in PDA animal models. Accordingly, the Dectin-1 signal pathway would be a reliable biomarker and treatment target for PDA, as well as other solid tumors.

Thus, aspects of the present disclosure relate to methods for treating or diagnosing a solid tumor such as PDAD by targeting Dectin-1, its ligand, or downstream components in the Dectin-1-mediated signaling pathway.

In one aspect, the present disclosure includes a method of treating a solid tumor (e.g., pancreatic ductal adenocarcinoma or colorectal cancer) in a subject by administering a therapeutically effective amount of a Dectin-1 antagonist, which can be an agent capable of suppressing the signaling pathway mediated by Dectin-1.

In some embodiments, the Dectin-1 antagonist is a Dectin-1 inhibitor, which inhibits the activity of Dectin-1 either directly or via reducing the level of Dectin-1. In some examples, a Dectin-1 inhibitor can be a small molecule compound that binds Dectin-1 and inhibits its activity. In other examples, a Dectin-1 inhibitor can be an anti-Dectin-1 antibody, which may not be conjugated with a second therapeutic agent such as an antigenic peptide or a TLR agonist. In yet other examples, a Dectin-1 inhibitor can be an interfering RNA (RNAi) that targets nucleic acids coding for Dectin-1 and thus blocks Dectin-1 expression.

In other embodiments, the Dectin-1 antagonist is an inhibitor of a Dectin-1 ligand (e.g., Galectin-9 or Galectin inhibitor), which inhibits the activity of the ligand. In some examples, a Galectin 9 inhibitor is a small molecule compound capable of binding to Galectin 9 and inhibits its activity. In other examples, the Galectin 9 inhibitor is an anti-Galectin 9 antibody, for example, an antibody that binds the cysteine-rich domain 1 (CRD1) or the cysteine-rich domain 2 (CRD2) of Galectin-9. In yet other examples, the Galectin 9 inhibitor is an interfering RNA (RNAi), which targets nucleic acids coding for Galectin 9 and thus suppresses the expression of Galectin 9.

In some embodiments, the Dectin-1 antagonist is an inhibitor of a downstream component in the Dectin-1 signaling pathway, for example the spleen tyrosine kinase (Syk). In some examples, the inhibitor of Syk may block phosphorylation of the Syk. Exemplary of such inhibitors include, but are not limited to, piceatannol, cerdulatinib (P505-15, PRT062607), fostamatinib disodium (R788), nilvadipine, ASN-002, MK-8457, entospletinib, GS-9876, TAK-659, TOP-1288, GSK-2646264, HMPL-523, SKIO-703, TOP-1630, AB-8779, CC-509, CVXL-0074, FF-10102, LAS-189386, PRT-2761, RO-9021, TAS-5567, TOP-1210, CG-103065, DNX-2000, Excellair, HM-029, HMPL-281, Jak3/Syk Dual Inhibitor, PRT-060318, PRT-2607, R-112, R-348, SKI-O-282, SKIO-592, R-333, R-343, C-13, RO9021, and R-406.

Any of the methods described herein may further comprise administering to the subject a second therapeutic agent, which may be an inhibitor of a checkpoint molecule, an activator of a co-stimulatory receptor, or an inhibitor of an innate immune cell target. Exemplary checkpoint molecules include PD-1, PD-L1, PD-L2, CTLA-4, LAG3, TIM-3, or A2aR. For example, the checkpoint inhibitor may be an anti-PD-1 antibody. Exemplary co-stimulatory receptors include OX40, GITR, CD137, CD40, CD27, or ICOS. Exemplary innate immune cell targets include KIR, NKG2A, CD96, TLR, or IDO.

In another aspect, the present disclosure provides a kit for treating a solid tumor, the kit comprising a first pharmaceutical composition that comprises a Dectin-1 antagonist (e.g., those described herein) and a second pharmaceutical composition that comprises the second therapeutic agent described herein, for example, an inhibitor of a checkpoint molecule, an activator of a co-stimulatory receptor, or an inhibitor of an innate immune cell target. In addition, described herein are pharmaceutical compositions which comprise the Dectin-1 antagonist, the second therapeutic agent, and a pharmaceutically acceptable carrier. Such a pharmaceutical composition can be for use in treating a solid tumor, for example PDA or CRC. Also within the scope of the present disclosure are uses of any of the Dectin-1 antagonists as described herein, either take alone or in combination with the second therapeutic agent, in manufacturing a medicament for use in treating a solid tumor, e.g., PDA or CRC.

An additional aspect of the present disclosure provides a method for analyzing a biological sample of a subject, comprising collecting a biological sample from a subject suspected of having pancreatic ductal adenocarcinoma (PDA) and measuring the level of Dectin-1 in the biological sample. Such a method may further comprise identifying whether or not the subject is at risk of PDA based on the level of Dectin-1 measured. An elevated level of Dectin-1 relative to that of a control subject (e.g., a subject of the same species and free of PDA) is indicative of presence or risk of PDA in the subject. If the subject is found to have, or be at risk of having, PDA, a treatment of PDA can be performed on that subject.

In some embodiments, the biological sample can be a blood sample containing cells and the level of Dectin-1 expressed on the cells can be measured. The biological sample may also be a tissue sample, which may be collected from a suspected tumor site in the subject. In some embodiments, the level of Dectin-1 in any of the methods described herein can be measured by an immune assay, which may involve an antibody specific to Dectin-1.

Further, the present disclosure provides an isolated antibody, which binds an epitope in CDR1 or CDR2 of a Galectin-9 polypeptide. In some embodiments, the Galectin-9 is human Galectin-9. Such an antibody may cross-reacts with a Galectin-9 polypeptide from another species (for example, mouse). In other embodiments, the isolated antibody binds human Galectin-9 more effectively than mouse Galectin-9 by a factor of at least 10-fold as determined under same assay conditions.

The details of one of more embodiments of the disclosure are set forth in the description below. Other features or advantages of the present disclosure will be apparent from the detailed description of several embodiments and also from the appended claims.

### BRIEF DESCRIPTION OF THE DRAWINGS

The following drawing forms part of the present specification and are included to further demonstrate certain aspects of the present disclosure, which can be better understood by reference to one or more of these drawings in combination with the detailed description of specific embodiments presented herein.

FIG. 1 shows high Dectin-1 expression in mouse and human PDA and Dectin-1 ligation acceleration of PDA progression. Panel A: photographs showing immunohistochemical (IHC) staining of frozen sections of 6 month-old KC; Dectin-1^{+/+} and KC; Dectin-1^{-/-} pancreata (scale bar=100 μm). Panel B diagrams showing representative contour plots and quantitative data of PDA-infiltrating and splenic leukocytes from KC and aged-matched WT mice (n=5/group) tested for expression of Dectin-1 in CD11c⁻Gr1⁻ CD11b⁺F4/80⁺ macrophages, Gr1⁺CD11b⁺ neutrophils and inflammatory monocytes, and CD11c⁺MHCII⁺ dendritic cells. Panel C diagrams showing representative contour plots and quantitative data from five mice. PDA-infiltrating and splenic leukocytes from mice bearing KPC-derived tumors were tested for expression of Dectin-1 compared with isotype control in CD11c⁻Gr1⁻CD11b⁺F4/80⁺ macrophages, Gr1⁺CD11b⁺ neutrophils and inflammatory monocytes, and CD11c⁺MHCII⁺ dendritic cells. Murine flow cytometry experiments were repeated more than 3 times. For each set of bars for M+, Neu/Mono, and DC, the bars from left to right are PDA, and Spleen. Panel D: charts showing flow cytometry of Dectin-1 expression in macrophages from normal pancreas compared with macrophages infiltrating orthotopic PDA tumors (n=5/group). In D, for each set of bars for M+, Neu/Mono, and DC, the bars from left to right are: PDA, PDA Spl, and WT Spl. Panel E diagrams showing representative contour plots and quantitative data from 5 PDA patients. In E, for each set of bars for CD14+, CD15+, and CD11c+, the bars from left to right are: PDA, and PBMC. Panels F to I: photos showing six week-old KC mice treated with the Dectin-1 ligands d-Zymosan, HKCA, or vehicle for 8 weeks before sacrifice (n=5/group). Panel F shows representative H&E (scale bar=100 μm) and Trichrome (scale bar=200 μm) stained sections. Panel G is a graph showing the percentage of ducts exhibiting normal morphology, ADM, graded PanIN lesions, or foci of invasive cancer are shown. Panel H is a graph showing the percentage of pancreatic area occupied by fibrotic tissues calculated based on trichrome staining. Panel I is a graph showing the percentage of pancreatic area occupied by normal acinar structures. Panel J is a photograph showing representative gross images of pancreatic tumors and a graph showing quantitative data of WT mice administered orthotopic KPC-derived tumor cells and serially treated with the Dectin-1 ligands d-Zymosan or HKCA or vehicle control. (n=5/group; scale bar=1 cm; *p<0.05; **p<0.01; ***p<0.001; ****p<0.0001).

FIG. 2 shows that Dectin-1 deletion or blockade is protective against PDA. Panel A photos showing representative H&E-stained sections and graphs of KC; Dectin-1^{+/+} (n=10) and KC; Dectin-1^{-/-} (n=6) mice sacrificed at 3, 6, or 9 months of life. The percentage of pancreatic area occupied by intact acinar structures, and the fractions of ductal structures exhibiting normal morphology, acino-ductal metaplasia (ADM), or graded PanIN I-III lesions were calculated (scale bar=200 μm). Panel B charts showing a Kaplan-Meier survival analysis comparing KC; Dectin-1^{+/+} (n=29) and KC; Dectin-1^{-/-} (n=41) mice (p=0.01). Panel C diagrams showing whole pancreas lysate from 3 month-old KC; Dectin-1^{+/+} and KC; Dectin-1^{-/-} mice assayed for expression of select oncogenic and tumor suppressor genes. Panel D diagrams showing representative H&E stained sections and a graph of pancreatic weights of six week-old KC; Dectin-1^{+/+} and KC; Dectin-1^{-/-} mice serially treated with the p-Syk inhibitor Piceatannol or vehicle for 8 weeks before sacrifice (n=5-10/group) (scale bar=200 μm). Each point represents data from a single mouse. Panel E is a graph showing the median fluorescence index (MFI) of WT mice bearing orthotopic PDA serially treated with the p-Syk inhibitor Piceatannol or vehicle for 3 weeks. Tumor-infiltrating APC were harvested and tested for p-Syk expression (n=5/group; *p<0.05; **p<0.01; ***p<0.001).

FIG. 3 shows that Dectin-1^{-/-} PDA-infiltrating monocytic cells exhibit diminished T cell suppressive properties. CD4⁺ and CD8⁺ T cell activation, respectively, were determined at 72h by ICOS expression (Panels A and B), the fraction of cells exhibiting the CD62L⁻CD44⁺ phenotype (Panels C and D), CD4⁺ T cell expression of IL-10 (Panel E), and CD8⁺ T cell co-expression of IFN-γ and TNF-α (Panel F). Experiments were performed in quadruplicate and repeated 3 times (*p<0.05; **p<0.01; ***p<0.001; ****p<0.0001).

FIG. 4 shows that Dectin-1 signaling regulates macrophage infiltration and phenotype in PDA. Panels A and B show immunohistochemical analyses and graphical representations of F4/80⁺ (Panel A) and Arg1⁺ (Panel B) macrophage infiltration (n=5/group) (scale bar=100 μm). Panel C shows flow cytometry analysis of CD11c⁻Gr1⁻CD11b⁺F4/80⁺ macrophages in the pancreata of three-month old KC; Dectin-1^{+/+} and KC; Dectin-1^{-/-} mice (n=5/group). Panel D shows CD11c⁻Gr1⁻CD11b⁺F4/80⁺ macrophage infiltration was determined on day 21 by flow cytometry. PDA-infiltrating macrophages were gated and tested for expression of MHC II (Panel E), CD206 (Panel F), TNF-α (Panel G), and iNOS (n=5/group). Panel H shows a representative contour plot and quantitative data of p-Syk expression in PDA-infiltrating macrophages in d-Zymosan-treated mice compared with vehicle-treated mice. Panel I shows a representative contour plat and quantitative data of tumor-infiltrating macrophages determined by flow cytometry on day 21. PDA-infiltrating macrophages were gated and tested for expression of CD206 (Panel J) and MHC II (Panel K). Experiments were repeated more than 3 times with similar results (n=5/group). Panel L is a graph depicting tumor volume recorded at serial intervals. This experiment was repeated twice with similar results (n=4/group; *p<0.05; **p<0.01; ****p<0.0001).

FIG. 5 shows that Dectin-1 signaling prevents immunogenic T cell differentiation in PDA. Panels A to C show flow cytometry data from KC; Dectin-1^{+/+} and KC; Dectin-1^{-/-} mice was assayed for CD44 (Panel A), Ox40 (Panel B), and PD-1 (Panel C) expression in CD4⁺ and CD8⁺ T cells (n=5/group). Panel D is a graph showing the CD8⁺:CD4⁺ T cell ratio (Panel D). Panel E is a graph showing the CD8⁺:CD4⁺ ratio determined on day 21 by flow cytometry. Panel F shows flow cytometry analyses of PDA-infiltrating CD8⁺ T cell expression of PD-1, T-bet, TNF-α, CD107a, and Granzyme B. Panel G shows flow cytometry analyses of PDA-infiltrating CD4⁺ T cell expression of CD44, CD107a, ICOS, T-bet, and TNF-α (n=5/group). Panel H shows tumor weights were measured on Day 21. Panel I shows the fraction of CD4⁺ T cells expressing IFN-γ, as determined by flow cytometry (n=5/group). All experiments were repeated at least twice (*p<0.05, **p<0.01, ***p<0.001).

FIG. 6 shows Galectin-9 is a novel Dectin-1 ligand in PDA. Panel A shows representative contour plots and quantitative data of PDA-infiltrating and splenic Gr1⁺CD11b⁺ neutrophils and inflammatory monocytes, CD11c⁻Gr1⁻CD11b⁺F4/80⁺ macrophages, and CD11c⁺MHCII⁺DC from mice harboring orthotopic KPC tumors tested for expression of Galectin-9. Gates are based on respective isotype control (not shown) (data from 5 mice shown). Panel B shows a representative contour plot of CD45⁻CD133⁺ pancreatic cancer cells from orthotopic KPC tumors tested for expression of Galectin-9 compared with isotype control. Representative data from >3 experiments is shown. Panel C shows representative contour plots of CD45⁺ and CD45⁻ cells from human PDA tumor tissue tested for expression of Galectin-9 compared with PBMC. Representative data from one of three patients are shown. Panel D is a representative confocal microscopy image of frozen sections of orthotopic KPC-derived pancreatic tumors co-stained for CD45 and Galectin-9 or isotype control (scale bar=50 μm). Panel E is a representative confocal microscopy image of frozen sections of orthotopic KPC-derived pancreatic tumors co-stained for CK19 and Galectin-9 or isotype control (scale bar=50 μm). Panel F shows flow cytometry data from bead-IgG Fc complexes incubated with recombinant Galectin-9 and then stained with fluorescently-conjugated anti-Galectin-9 and tested for fluorescence. Panels G and H are graphs quantifying Galectin-9-Dectin-1 binding using Galectin-9-coated ELISA plates incubated with increasing doses of murine (Panel G) or human (Panel H) Dectin-1 IgG Fc or control IgG Fc. Averages of triplicates are shown. ELISA assays was repeated twice with similar results. Panel I is a graph quantifying the results of Galectin-3, Galectin-4, and Galectin-9 coated ELISA assays. Averages of triplicates are shown. Panel J is a Western blot, showing the results of Dectin-1 ligand precipitation in pancreatic tissue extract probing for Galectin-9. This assay was repeated twice with similar results. Panel K is a gel showing Dectin-1 IgG Fc treated with either buffer or PNGase F, incubated with recombinant mouse Galectin-9. Treated and control samples were analyzed by SDS-PAGE and gels were stained with Coomassie Blue. This experiment was repeated twice with similar results. Panel L is two graphs and representative histogram overlays showing Syk phosphorylation of WT and Dectin-1^{-/-} macrophages were treated with Galectin-9 (10 ug/ml) for 3 hours. Data from 5 separate experiments are shown. Panel M is a graph showing Dectin-1 activation measured by detection of secreted embryonic alkaline phosphatase. This assay was performed in triplicate (*p<0.05; **p<0.01; ****p<0.0001).

FIG. 7 shows Dectin-1 expression in murine and human PDA. Panels A and B show representative confocal microscopy images of frozen sections of 6 month-old KC; Dectin-1^{+/+} and KC; Dectin-1^{-/-} pancreata co-stained for Dectin-1 and CD45 (Panel A) or Dectin-1 and CK19 (Panel B)(scale bar=50 nm). Panels C and D show representative confocal microscopy images of frozen sections of orthotopic KPC-derived pancreatic tumors co-stained for Dectin-1 and CD45 (Panel C) or Dectin-1 and CK19 (Panel D)(scale bar=50 nm). Panel E shows a flow cytometry analysis of KPC-derived tumor cells tested for expression of Dectin-1 by compared with isotype control. Panels F and G show representative confocal microscopy images of frozen sections from human PDA tested for co-expression of Dectin-1 and EPCAM (Panel F) and Dectin-1 and CD11b (Panel G) (scale bar=50 μm). Panel H is a graph showing the percent of Dectin-1-positive bone marrow-derived macrophages (BMDM) after eight days of co-culture with the indicated cells, supernatant, or cytokine. Experiments were repeated twice (*p<0.05, **p<0.01, ***p<0.001, ****p<0.0001).

FIG. 8 shows evidence for Dectin-1 signaling and Dectin-1 ligand expression in PDA. Panel A is a composite Western blot depicting the presence or absence of phosphorylated and non-phosphorylated signaling intermediates downstream of Dectin-1 activation. Proteins were equally loaded. Ponceau staining is shown. Panel B is images and a graph that show the expression of p-Syk by IHC. Data was quantified by examining 10 HPFs per slide (n=3/group) (scale bar=100 μm). Panel C shows flow cytometry screening for p-Syk. Data was quantified based on results from 5 mice. Panel D shows a confocal microscopy image of frozen sections of pancreata from 6 month-old KC mice co-stained using αCD68 and a human IgG Fc-conjugated Dectin-1 fusion protein (scale bar=50 μm). Panel E shows the results of flow cytometry in 6 month-old KC mice using the IgG Fc-conjugated Dectin-1 fusion protein and a fluorescently conjugated α-human IgG. Data was quantified based on results from 5 mice. Panel F shows the results of flow cytometry in orthotopic KPC tumor-bearing mice tested for expression of Dectin-1 agonists. Quantitative data from 5 mice is shown. CD45⁻CD133⁺ epithelial cells from KC (Panel G) and orthotopic KPC (Panel H) tumors were gated and tested for expression of Dectin-1 ligands compared with isotype control. Representative data are shown. Panel I shows representative flow cytometry data of KPC-derived tumor cells cultured in vitro tested for Dectin-1 ligand expression using the human IgG Fc-conjugated Dectin-1 fusion protein and fluorescently-conjugated anti-human IgG. All experiments were reproduced at least twice (**p<0.01; ***p<0.001; ****p<0.0001).

FIG. 9 shows that Dectin-1 ligation is associated with accelerated epithelial cell proliferation and Dectin-1 deletion mitigates peri-tumoral fibrosis in PDA. Panel A shows immunohistochemical images of KC mice treated with Dectin-1 ligands and a graph representing the rate of epithelial cell proliferation (n=5/group; scale bar=100 μm). Panel B shows representative Trichrome-stained sections of KC (n=10) and KC; Dectin-1^{-/-} (n=6) mice sacrificed at 3, 6, or 9 months of life. The fraction of fibrotic pancreatic area was calculated for each cohort (scale bar=200 μm; *p<0.05, **p<0.01, ***p<0.001, ****p<0.0001).

FIG. 10 shows Dectin-1 ligation or knockdown in PDA cells does not alter tumor phenotype whereas Dectin-1 deletion in inflammatory cells is protective. Panels A to C are graphs depicting cellular proliferation (Panel A), expression of MCP-1 (Panel B), and expression of IL-10 (Panel C) in cell culture supernatant from KPC-derived tumor cells. Panel D shows a Western blot used to confirm the efficiency of Dectin-1 knockdown using shRNA. Panel E shows representative images and quantification of tumors from WT mice orthotopically implanted with KPC-derived tumor cells that had been treated with shRNA directed against Dectin-1 or with control scrambled shRNA (scale bar=1 cm; n=5/group). Panel F is a graph showing cellular proliferation in vitro using the XTT assay. Panel G shows representative gross images and quantification of in vivo cellular proliferation (scale bars=1 cm for tumor pictures and 100 μm for H&E; n=7/group). Panel H show representative gross images and a graph of average tumor weights (scale bar=1 cm; n=5/group; ***p<0.001). Panel I shows a Kaplan-Meier analysis of WT and Dectin-1^{-/-} mice (n=8-10/group; p=0.02). Panel J shows representative H&E-stained pancreatic sections and the calculated fractions of ductal structures exhibiting normal morphology, acino-ductal metaplasia (ADM), or graded PanIN I-III lesions (n=5/group; scale bar=100 μm). Panel K shows a Kaplan-Meier survival analysis of human PDA tumors with high vs. low tertile levels of Dectin-1 expression using the UCSC RNAseq database (p=ns). Panel L is a graph showing the association between expression of Dectin-1 and CD11b (ITGAM) in human PDA utilizing the UCSC RNAseq database (***p<0.001, ****p<0.0001).

FIG. 11 shows that Dectin-1 deletion abrogates the immune-suppressive properties in PDA-infiltrating macrophages and results in expansion of antigen-restricted cytotoxic T cells. Panel A shows flow cytometry data and a graph from CD4⁺ splenic T cells that were either unstimulated, or stimulated with αCD3/αCD28 alone or in co-culture with PDA-infiltrating CD11c⁻Gr1⁻CD11b+F4/80⁺ macrophages from WT or Dectin-1^{-/-} hosts. Panel B shows flow cytometry from the same experiment as in Panel A, but with CD8⁺ splenic T cells. Experiments were repeated twice with similar results using TAMs harvested from 5 animals per group. Panel C is a graph depicting T cell activation in CD8⁺ splenic T cells after no stimulation, or 72 hours following stimulation with αCD3/αCD28 alone or in co-culture with PDA-infiltrating Gr1⁺CD11b⁺ neutrophils and inflammatory monocytes harvested from WT or Dectin-1^{-/-} hosts. This experiment was repeated twice using 5 replicates. Panel D is a heat map is shown based on results of a nanostring array testing inflammatory gene expression in orthotopic PDA tissues in WT and Dectin-1^{-/-} mice (n=3/group). The fraction of CD11c⁺MHCII⁺DC in orthotopic PDA tumors in WT and Dectin-1^{-/-} mice (Panel E) and DC expression of CD86, CD103, and 1-6 (Panel F) were determined on day 21 (n=5/group). Panels G and H: WT or Dectin-1^{-/-} mice were orthtopically implanted with KPC-derived tumor cells engineered to express OVA. On day 21, tumors were harvested and the percentage of CD8⁺ T cells among all CD3⁺ T cells (Panel G) and the percentage of OVA Pentamer⁺ cells among all CD8⁺ T cells (Panel H) were determined by flow cytometry (n=4-5/group; *p<0.05; **p<0.01; ***p<0.001).

FIG. 12 shows that Dectin-1 signaling in PDA-infiltrating TAMs induces an immune-suppressive T cell phenotype which promotes tumor growth. Panel A shows representative contour plots and quantitative data of tumor-infiltrating CD4⁺ T cells interrogated on day 21 for expression of T-bet, TNF-α, IL-5, IL-10, and IL-13 compared with isotype control. Panel B shows representative contour plots and quantitative data of CD8⁺ T cells were tested for T-bet and TNF-α expression. Experiments were repeated 3 times using 5 mice per group. Mice were sacrificed on day 21 (n=5/group). Panel C shows representative contour plots and quantification data of tumor-infiltrating CD4⁺ T cell. Panel D shows representative contour plots and quantification data of tumor-infiltrating CD8⁺ T cell adoption of an activated CD44+CD62L-phenotype. Experiments were repeated twice with similar results. Panel E: Panel E shows representative gross images of tumors and quantitative data. Mice were sacrificed at 21 days and pancreatic tumors were weighed (n=5/group). Panels F and G: Dectin-1^{-/-} mice were challenged with orthotopic PDA and serially treated with neutralizing αCD4 (Panel F) or αCD8 (Panel G) mAbs or isotype. Mice were sacrificed at 21 days and pancreatic tumors weighed (n=5/group; *p<0.05; **p<0.01).

FIG. 13 shows that Galectin-9 promotes pancreatic oncogenesis and immune suppression in PDA. Panel A shows a Kaplan-Meier survival analysis of WT mice orthotopically implanted with KPC-derived tumor cells and serially treated with a neutralizing α-Galectin-9 mAb or isotype control (n=8-10/group; p=0.005). Panel B shows a Kaplan-Meier survival analysis of WT mice orthotopically implanted with KPC-derived tumor cells and serially treated with a neutralizing α-Galectin-9 mAb or isotype control beginning on day 8 after tumor implantation (n=20/group; p=0.0013). Panel C shows a Kaplan-Meier survival analysis of human PDA patients with respective high or low tertile levels of Gal9 expression (p=0.07). Panel D is a graph showing the percentage increase or regression in tumor size compared between day 8 and day 14 (n=10/group). Panel E shows representative images and quantitative data on tumor weights (scale bar=1 cm; n=5/group). Panel F: Shows quantitative data on tumor weights are shown (n=5/group). Panel G shows flow cytometry data from day 21, when CD11c⁻Gr1⁻CD11b⁺F4/80⁺ TAMs were tested for expression of MHC II and TNF-α (n=5/group). Panel H shows representative H&E staining of tumor sections (scale bar=100 μm). Panel I is a graph showing the fraction of TAMs among CD45⁺ tumor-infiltrating leukocytes in each cohort. Panel J is a graph showing expression of MHC II in TAMs in each cohort. Panel K is a graph showing the CD8/CD4 ratio on day 21 and day 42. Panel L is a graph showing CD8⁺ T cell expression of IFN-γ on day 21 and day 42. Panel M is a graph showing T-bet on day 21 and day 42. In vivo experiments were repeated at least twice (*p<0.05; **p<0.01).

FIG. 14 shows the pro-tumorigenic effects of the Dectin-1-Galectin-9 signaling axis in PDA. Panel A is representative contour plots and a graph showing CD44 expression in PDA-infiltrating CD4⁺ T cells on day 21. Panel B is representative contour plots and a graph showing PDA-infiltrating CD8⁺ T cells tested for expression of T-bet. This experiment was repeated twice (n=5/group; *p<0.05; **p<0.01; ***p<0.001). Panel C is a schematic depicting immune-suppressive effects of Dectin-1 signaling or immunogenic consequences of targeting Dectin-1 in PDA.

FIG. 15 shows schematics of three Galectin-9 isoforms and an alignment (SEQ ID NO:3) of human (SEQ ID NO:1) and mouse (SEQ ID NO:2) sequences. The CRD1 region is presented in orange (numbers 1-148 for human, 1-147 for mouse) and the CRD2 region is presented in green (numbers 227-355 for human, and 225-353 for mouse).

FIG. 16 is a gel showing purification of human and mouse Galectin-9 CRD2.

FIG. 17 is a graph showing that anti-Galectin-9 antibody binds both human and mouse Galectin-9 CRD2.

### DETAILED DESCRIPTION OF DISCLOSURE

The progression of pancreatic oncogenesis requires immune-suppressive inflammation in cooperation with oncogenic mutations. However, the drivers of intra-tumoral immune tolerance are uncertain. Dectin-1 is an innate immune receptor critical in anti-fungal immunity, but its role in sterile inflammation and oncogenesis is not well-defined. Further, non-pathogen-derived ligands for Dectin-1 have not been characterized.

The present disclosure is based, at least in part, on the unexpected discoveries that Dectin-1 is highly expressed on macrophages in pancreatic ductal adenocarcinoma (PDA); Dectin-1 ligation accelerated PDA; and Dectin-1 deletion or blockade of its downstream signaling was protective of PDA. Further, Dectin-1 was found to ligate the lectin Galectin-9 in the PDA tumor microenvironment, resulting in tolerogenic macrophage programming and adaptive immune suppression. Upon interruption of the Dectin-1-Galectin-9 axis, CD4⁺ and CD8⁺ T cells—which are dispensable to PDA progression in hosts with an intact signaling axis—become reprogrammed into indispensable mediators of anti-tumor immunity. These data suggest that targeting Dectin-1 signaling is an attractive strategy for the immunotherapy of solid tumors such as PDA.

Accordingly, described herein are methods for treating or diagnosing solid tumors such as PDA, via targeting Dectin-1, a ligand thereof, or a downstream component of the Dectin-1 signaling pathway.

### Methods of Treatment

The present disclosure provides methods of treating a solid tumor in a subject by administering a therapeutically effective amount of a Dectin-1 antagonist.

### (i) Dectin-1 Antagonists

The term “Dectin-1 antagonist” as used herein refers to a compound that is capable of suppressing the signaling pathway mediated by Dectin-1. A Dectin-1 antagonist for use in the method described herein may be an inhibitor that targets Dectin-1, a ligand of Dectin-1 (e.g., Galectin-9), or a downstream component of the Dectin-1 mediated signaling pathway (e.g., Syk or PLCγ). An inhibitor of a target refers to an agent capable of reducing the bioactivity of the target (e.g., by at least 30%, 40%, 50%, 80%, 90% or above) or eliminating the bioactivity of the target (e.g., no biologically significant activity can be detected in a conventional assay in the presence of the inhibitor). It may directly interact with the target and inhibits its bioactivity. Alternatively, it may reduce the expression level of the target, leading to the decrease of the bioactivity of the target.

Dectin-1, also known as C-type lectin domain family 7 member A, is a member of the C-type lectin family of pattern recognition receptors (PRRs) and is required for inflammatory responses to fungal pathogens. Various isoforms of Dectin-1 are identified, all of which are within the scope of the present disclosure. Examples include human isoform a (GenBank accession no. NP_922938.1), human isoform 6 (GenBank accession no. NP_072092.2), human isoform c (GenBank accession no. NP_922939.1), human isoform d (GenBank accession no. NP_922940.1), human isoform e (GenBank accession no. NP_922941.1) and human isoform f (GenBank accession no. NP_922945.1). Other examples of Dectin-1 (e.g., those of other mammals, for example, mouse, rat, and monkey) are well known in the art; their amino acid sequence information can be obtained from GenBank.

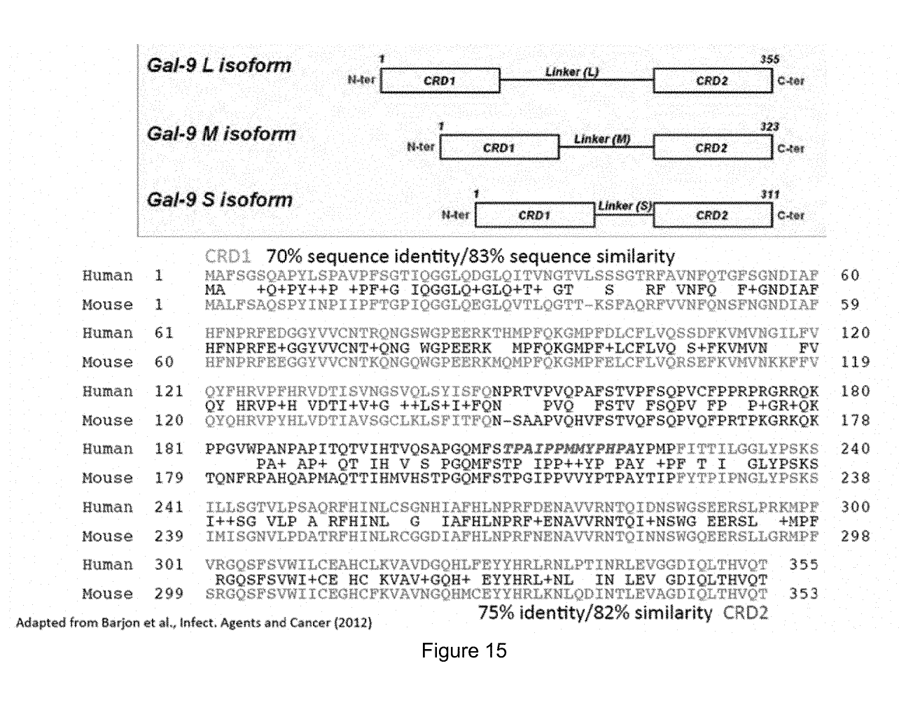

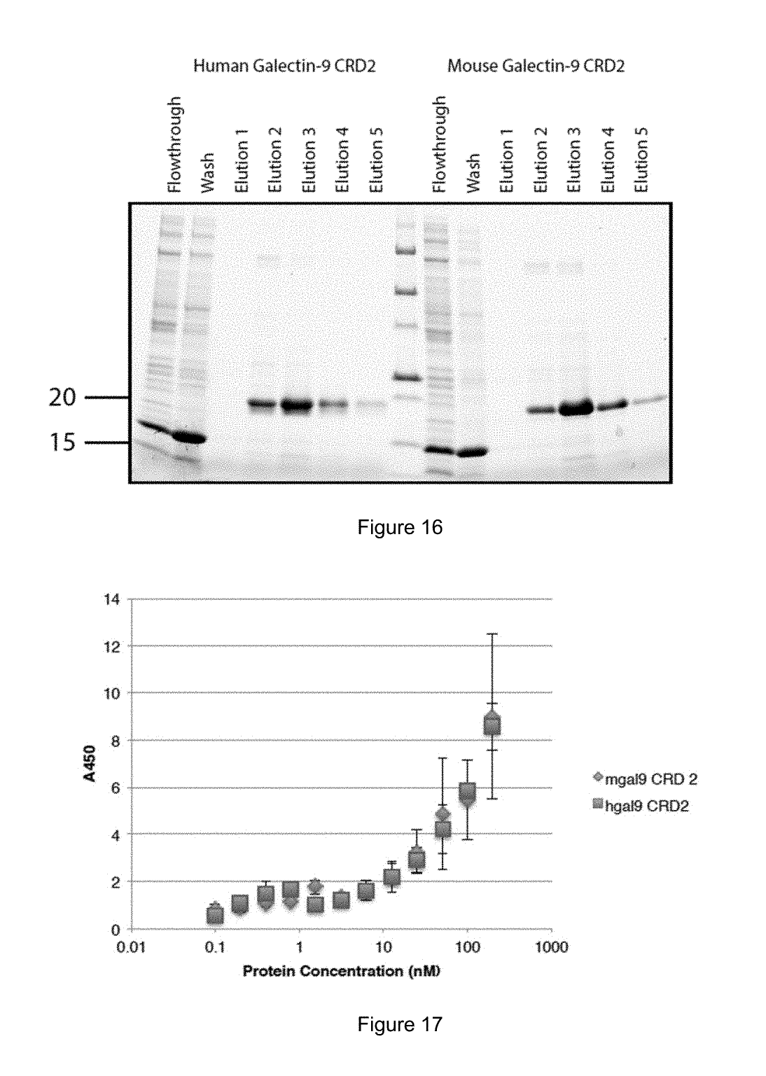

Galectin-9 is a member of the Galectins family, which has high binding affinity to β-galactoside sugars. Galectin-9 has three different isoforms which differ in the length of the linker region. FIG. 15. Exemplary human Galectin-9 polypeptides include those described under GenBank accession no. 000182.2, GenBank accession no. BAB83624.1, and GenBank accession no. BAB83623.1. In some examples, the anti-Galectin-9 antibodies described herein binds the CRD1 domain or the CRD2 domain as illustrated in FIG. 15.

The Dectin-1 signaling pathway is well characterized, which uses the spleen tyrosine kinase (SYK) as a downstream kinase regulator. Brown, Nature Reviews Immunology 6, 33-43 (January 2006). In addition, phospholipase Cγ (PLCγ) has been shown to play an essential role in Dectin-1-mediated calcium flux as well. Xu et al., J Biol Chem., 284, 7038-7046 (2009).

A. Antibodies Suppressing the Dectin-1 Signaling Pathway

In some embodiments, the Dectin-1 antagonists described herein are antibodies that bind Dectin-1, a Dectin-1 ligand such ad Galectin-9, or a downstream component of the Dectin-1 signaling pathway and thus neutralize the activity of the target antigen.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

In some embodiments, an antibody as described herein can bind and inhibit a target antigen (e.g., Dectin-1 or Galectin-9) by at least 50% (e.g., 60%, 70%, 80%, 90%, 95% or greater). The apparent inhibition constant (Ki^{app} or K_{i,app}), which provides a measure of inhibitor potency, is related to the concentration of inhibitor required to reduce enzyme activity and is not dependent on enzyme concentrations. The inhibitory activity of the antibody described herein can be determined by routine methods known in the art.

The K_{i,}^{app} value of an antibody may be determined by measuring the inhibitory effect of different concentrations of the antibody on the extent of the reaction (e.g., enzyme activity); fitting the change in pseudo-first order rate constant (ν) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of K_{i,}^{app} versus substrate concentration.

$$v = A \cdot \frac{\begin{matrix}( [ E ] - [ I ] - {K}_{i}^{app} ) + \\ \sqrt{{( [ E ] - [ I ] - {K}_{i}^{app} )}^{2} + 4 [ E ]} \cdot {K}_{i}^{app}\end{matrix}}{2} \qquad ( Equation 1 )$$

where A is equivalent to νₒ/E, the initial velocity (νₒ) of the enzymatic reaction in the absence of inhibitor (1) divided by the total enzyme concentration (E).

In some embodiments, the antibody described herein may have a Ki^{app} value of 1000, 900, 800, 700, 600, 500, 400, 300, 200, 100, 50, 40, 30, 20, 19, 18, 17, 16, 15, 14, 13, 12, 11, 10, 9, 8, 7, 6, 5 pM or less for the target antigen or antigen epitope as described herein. In some embodiments, the antibody may have a lower Ki^{app} for a first target (e.g., a human Dectin-1 or human Galectin-9) relative to a second target (e.g., a mouse Dectin-1 or a mouse Galectin-9). Differences in Ki^{app} (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some examples, the antibody inhibits a first antigen (e.g., a first protein in a first conformation or mimic thereof) better relative to a second antigen (e.g., the same first protein in a second conformation or mimic thereof, or a second protein). In some embodiments, any of the antibodies may be further affinity matured to reduce the Ki^{app} of the antibody to the target antigen or antigenic epitope thereof.

The antibodies described herein can be murine, rat, human, or any other origin (including chimeric or humanized antibodies). Such antibodies are non-naturally occurring, i.e., would not be produced in an animal without human act (e.g., immunizing such an animal with a desired antigen or fragment thereof).

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, and/or six) which are altered with respect to the original antibody, which are also termed one or more CDRs “derived from” one or more CDRs from the original antibody. Humanized antibodies may also involve affinity maturation.

In another example, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the antibodies described herein specifically bind to the corresponding target antigen or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (e.g., Dectin-1 or Galectin-9) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen. In some embodiments, the antibodies described herein specifically bind to a Dectin-1 polypeptide, for example, human Dectin-1. In some embodiments, the antibodies described herein specifically bind to a Galectin-9 polypeptide, for example, human Galectin-9 or an epitope therein (e.g., the CRD1 or CRD2 regions therein).

In some embodiments, an antibody as described herein has a suitable binding affinity for the target antigen (e.g., Dectin-1 or Galectin-9) or antigenic epitopes thereof (e.g., CRD1 or CRD2 of Galectin-9). As used herein, “binding affinity” refers to the apparent association constant or K_{A}. The K_{A} is the reciprocal of the dissociation constant (K_{D}). The antibody described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some embodiments, any of the antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Antibodies capable of binding to Dectin-1, Galectin-9, or a downstream component of the Dectin-1 signaling pathway as described herein can be made by any method known in the art. See, for example, Harlow and Lane, (1998) Antibodies: A Laboratory Manual, Cold Spring Harbor Laboratory, New York.

In some embodiments, antibodies specific to a target antigen as described herein can be made by the conventional hybridoma technology. The full-length target antigen or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the monoclonal antibodies specific to the target antigens described herein. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies capable of interfering with the Dectin-1 signaling. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups, can yield a population of antibodies (e.g., monoclonal antibodies).

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy in inhibiting the activity of the target antigen. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, Calif.) and HuMAb-Mouse® and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Alternatively, antibodies capable of binding to the target antigens as described herein may be isolated from a suitable antibody library via routine practice, for example, using the phage display, yeast display, ribosomal display, or mammalian display technology known in the art.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of“chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of VH and VL of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human VH and VL chains having amino acid sequences that are homologous to those of the parent non-human antibody are identified from any antibody gene database using the parent VH and VL sequences as search queries. Human VH and VL acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a target antigen can be identified from the library following routine procedures. Positive clones can be subjected to further screening to identify those that inhibit the activity of the target antigen.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of the target polypeptide have been replaced (swapped) with sequences from a closely related, but antigenically distinct protein (such as another member of the neurotrophin protein family). By assessing binding of the antibody to the mutant target antigen, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some examples, an antibody as described herein can be prepared by the conventional recombinant technology using a suitable host cell, for example, a mammalian cell line (e.g., CHO cells).

Any of the antibodies capable of binding to Lectin-1, Galectin-9, or a downstream component of the Dectin-1 signaling pathway is within the scope of the present disclosure. The antibodies for use in the methods described herein may be free antibodies, for example, not be conjugated with a second therapeutic agent (e.g., antigenic peptides or TLR agonists), including those that are capable of activating immune cells such as dendritic cells.

B. Other Dectin-1 Antagonists

In addition to the antibodies described herein, Dectin-1 antagonists may be antisense nucleic acid molecules capable of blocking or decreasing the expression of Dectin-1, Galectin-9, or a downstream component of the Dectin-1 signaling pathway (e.g., Syk). Nucleotide sequences encoding those target molecules are known and are readily available from publicly available databases. See above disclosures. It is routine to prepare antisense oligonucleotide molecules that will specifically bind a target mRNA without cross-reacting with other polynucleotides. Exemplary sites of targeting include, but are not limited to, the initiation codon, the 5′ regulatory regions, the coding sequence and the 3′ untranslated region. In some embodiments, the oligonucleotides are about 10 to 100 nucleotides in length, about 15 to 50 nucleotides in length, about 18 to 25 nucleotides in length, or more. The oligonucleotides can comprise backbone modifications such as, for example, phosphorothioate linkages, and 2′-0 sugar modifications well known in the art.

Alternatively, the expression and/or release of any of the target antigens described herein can be decreased using gene knockdown, morpholino oligonucleotides, small interfering RNA (siRNA or RNAi), microRNA or ribozymes, methods that are well-known in the art. RNA interference (RNAi) is a process in which a dsRNA directs homologous sequence-specific degradation of messenger RNA. In mammalian cells, RNAi can be triggered by 21-nucleotide duplexes of small interfering RNA (siRNA) without activating the host interferon response. The dsRNA used in the methods disclosed herein can be a siRNA (containing two separate and complementary RNA chains) or a short hairpin RNA (i.e., a RNA chain forming a tight hairpin structure), both of which can be designed based on the sequence of the target gene. Alternatively, it can be a microRNA.

Optionally, a nucleic acid molecule to be used in the method described herein (e.g., an antisense nucleic acid, a small interfering RNA, or a microRNA) as described above contains non-naturally-occurring nucleobases, sugars, or covalent internucleoside linkages (backbones). Such a modified oligonucleotide confers desirable properties such as enhanced cellular uptake, improved affinity to the target nucleic acid, and increased in vivo stability.

In one example, the nucleic acid has a modified backbone, including those that retain a phosphorus atom (see, e.g., U.S. Pat. Nos. 3,687,808; 4,469,863; 5,321,131; 5,399,676; and 5,625,050) and those that do not have a phosphorus atom (see, e.g., U.S. Pat. Nos. 5,034,506; 5,166,315; and 5,792,608). Examples of phosphorus-containing modified backbones include, but are not limited to, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having 3′-5′ linkages, or 2′-5′ linkages. Such backbones also include those having inverted polarity, i.e., 3′ to 3′,5′ to 5′ or 2′ to 2′ linkage. Modified backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In another example, the nucleic acid used in the disclosed methods includes one or more substituted sugar moieties. Such substituted sugar moieties can include one of the following groups at their 2′ position: OH; F; O-alkyl, S-alkyl, N-alkyl, O-alkenyl, S-alkenyl, N-alkenyl; O-alkynyl, S-alkynyl, N-alkynyl, and O-alkyl-O-alkyl. In these groups, the alkyl, alkenyl and alkynyl can be substituted or unsubstituted C₁ to C₁₀ alkyl or C₂ to C₁₀ alkenyl and alkynyl. They may also include at their 2′ position heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide. Preferred substituted sugar moieties include those having 2′-methoxyethoxy, 2′-dimethylaminooxyethoxy, and 2′-dimethylaminoethoxyethoxy. See Martin et al., Helv. Chim. Acta, 1995, 78, 486-504.

In yet another example, the nucleic acid includes one or more modified native nucleobases (i.e., adenine, guanine, thymine, cytosine and uracil). Modified nucleobases include those described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition, 1991, 30, 613, and Sanghvi, Y. S., Chapter 15, Antisense Research and Applications, pages 289-302, CRC Press, 1993. Certain of these nucleobases are particularly useful for increasing the binding affinity of the antisense oligonucleotide to its target nucleic acid. These include 5-substituted pyrimidines, 6-azapyrimidines and N-2, N-6 and 0-6 substituted purines (e.g., 2-aminopropyl-adenine, 5-propynyluracil and 5-propynylcytosine). See Sanghvi, et al., eds., Antisense Research and Applications, CRC Press, Boca Raton, 1993, pp. 276-278).

Any of the nucleic acids can be synthesized by methods known in the art. See, e.g., Caruthers et al., 1992, Methods in Enzymology 211, 3-19, Wincott et al., 1995, Nucleic Acids Res. 23, 2677-2684, Wincott et al., 1997, Methods Mol. Bio. 74, 59, Brennan et al., 1998, Biotechnol Bioeng., 61, 33-45, and Brennan, U.S. Pat. No. 6,001,311. It can also be transcribed from an expression vector and isolated using standard techniques.

In other embodiments, the Dectin-1 antagonists described herein can be non-antibody compounds that directly or indirectly reduce, inhibit, neutralize, or abolish the biological activity of Dectin-1, a ligand thereof such as Galectin-1, or a downstream component such as Syk. Such an inhibitory compound should exhibit any one or more of the following characteristics: (a) binds to the target molecule and inhibits its biological activity and/or downstream pathways mediated by Dectin-1 signaling function; (b) prevents, ameliorates, or treats any aspect of tumor growth, including, e.g., enhance immune surveillance by, e.g., CD4+ and/or CD8+ T cells, and/or suppressing the activity of γδ T cells infiltrated in to the tumor microenvironment.

In some embodiments, an inhibitory compound can be a Dectin-1 mutant or a Galectin-9 mutant, which can bind to a cell surface Galectin-9 or Dectin-1, respectively, but cannot elicit signal transduction. Such a mutant may block binding of wild type Dectin-1 to a wild-type Galectin-9, thus suppressing the Dectin-1 signal transduction.

In other embodiments, the inhibitory compounds described herein are small molecules, which can have a molecular weight of about any of 100 to 20,000 daltons, 500 to 15,000 daltons, or 1000 to 10,000 daltons. Libraries of small molecules are commercially available. For example, Piceatannol, P505-15 (PRT062607) and fostamatinib disodium (R788) can be used in any of the methods described herein for blocking phosphorylation of Syk, thereby suppressing the Dectin-1 signaling.

The above-mentioned small molecules can be obtained from compound libraries. The libraries can be spatially addressable parallel solid phase or solution phase libraries. See, e.g., Zuckermann et al. J. Med. Chem. 37, 2678-2685, 1994; and Lam Anticancer Drug Des. 12:145, 1997. Methods for the synthesis of compound libraries are well known in the art, e.g., DeWitt et al. PNAS USA 90:6909, 1993; Erb et al. PNAS USA 91:11422, 1994; Zuckermann et al. J. Med. Chem. 37:2678, 1994; Cho et al. Science 261:1303, 1993; Carrell et al. Angew Chem. Int. Ed. Engl. 33:2059, 1994; Carell et al. Angew Chem. Int. Ed. Engl. 33:2061, 1994; and Gallop et al. J. Med. Chem. 37:1233, 1994. Libraries of compounds may be presented in solution (e.g., Houghten Biotechniques 13:412-421, 1992), or on beads (Lam Nature 354:82-84, 1991), chips (Fodor Nature 364:555-556, 1993), bacteria (U.S. Pat. No. 5,223,409), spores (U.S. Pat. No. 5,223,409), plasmids (Cull et al. PNAS USA 89:1865-1869, 1992), or phages (Scott and Smith Science 249:386-390, 1990; Devlin Science 249:404-406, 1990; Cwirla et al. PNAS USA 87:6378-6382, 1990; Felici J. Mol. Biol. 222:301-310, 1991; and U.S. Pat. No. 5,223,409).

C. Identification of Dectin-1 Antagonists

Dectin-1 antagonists can be identified or characterized using methods known in the art, whereby reduction, amelioration, or neutralization of the Dectin-1 biological activity is detected and/or measured. For example, an ELISA-type assay may be suitable for qualitative or quantitative measurement of Dectin-1 mediated kinase activation by measuring the phosphorylation of proteins activated through a Dectin-1 cascade, for example, Syk.

The Dectin-1 antagonists can also be identified by incubating a candidate agent with Dectin-1, Galectin-9, or a downstream component and monitoring any one or more of the following characteristics: (a) binding between Dectin-1 and Galectin-9 and inhibiting the signaling transduction mediated by the binding; (b) preventing, ameliorating, or treating any aspect of a solid tumor bone fracture; (c) blocking or decreasing Dectin-1 activation; (d) increasing clearance of Dectin-1, Galectin-9, or the downstream component; (e) inhibiting (reducing) synthesis, production or release of any of the target antigen.

### (ii) Pharmaceutical Compositions

Any of the Dectin-1 antagonists (e.g., antibodies, antisense nucleic acids, polypeptide mutants, and small molecule inhibitors) as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The Dectin-1 antagonists may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(v nylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

(iii) Therapeutic Applications

To practice the method disclosed herein, an effective amount of the Dectin-1 antagonist described herein, formulated in a suitable pharmaceutical composition as also described herein, can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a solid tumor, such as pancreatic duct adenocarincoma (PDA), colorectal cancer (CRC), melanoma, breast cancer, lung cancer (for example, non-small cell lung cancer, NSCLC, and small cell lung cancer, SCLC), upper and lower gastrointestinal malignancies (including, but not limited to, esophageal, gastric, and hepatobiliary cancer), squamous cell head and neck cancer, genitourinary, and sarcomas. A subject having a solid tumor can be identified by routine medical examination, e.g., laboratory tests, organ functional tests, CT scans, or ultrasounds. Such a subject may also be identified by the diagnostic method described herein. A subject suspected of having any of such target disease/disorder might show one or more symptoms of the disease/disorder. A subject at risk for the disease/disorder can be a subject having one or more of the risk factors for that disease/disorder. In some embodiments, the subject to be treated by the method described herein may be a human cancer patient who has undergone or is subjecting to an anti-cancer therapy, for example, chemotherapy, radiotherapy, immunotherapy, or surgery.

As used herein, “an effective amount” refers to the amount of each active agent required to confer therapeutic effect on the subject, either alone or in combination with one or more other active agents. In some embodiments, the therapeutic effect is reduced Dectin-1 signaling, including reduced Dectin-1 activity or expression, reduced Galectin-9 activity or expression, reduced phosphorylation of Syk or reduced expression of Syk, or enhanced anti-tumor immunity via, e.g., enhanced αβ T cell activity and/or reduced activity of γδ T cells infiltrated into the TME. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for a Dectin-1 antagonist such as an antibody as described herein may be determined empirically in individuals who have been given one or more administration(s) of the antagonist. Individuals are given incremental dosages of the antagonist. To assess efficacy of the antagonist, an indicator of the disease/disorder can be followed.

Generally, for administration of any of the antibodies described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 gig/mg to about 2 mg/kg (such as about 3 gig/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

Small molecule antagonists can be administered using any means known in the art, including inhalation, intraperitoneally, intravenously, intramuscularly, subcutaneously, intrathecally, intraventricularly, orally, enterally, parenterally, intranasally, or dermally. In general, when the Dectin-1 antagonist described herein is a small molecule, it will be administered at the rate of 0.1 to 300 mg/kg of the weight of the patient divided into one to three or more doses. For an adult patient of normal weight, doses ranging from 1 mg to 5 g per dose can be administered.

For the purpose of the present disclosure, the appropriate dosage of a Dectin-1 antagonist as described herein will depend on the specific antagonist employed, the type and severity of the solid tumor, whether the antagonist is administered for preventive or therapeutic purposes, previous therapy, the patient's clinical history and response to the antagonist, and the discretion of the attending physician. Typically the clinician will administer an antibody, until a dosage is reached that achieves the desired result. In some embodiments, the desired result is a decrease in thrombosis. Methods of determining whether a dosage resulted in the desired result would be evident to one of skill in the art.

Administration of one or more antagonists can be continuous or intermittent, depending, for example, upon the recipient's physiological condition, whether the purpose of the administration is therapeutic or prophylactic, and other factors known to skilled practitioners. The administration of the antagonist may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

In some embodiments, the antibodies described herein are administered to a subject in need of the treatment at an amount sufficient to inhibit the Dectin-1 signaling by at least 20% (e.g., 30%, 40%, 50%, 60%, 70%, 80%, 90% or greater) in vivo. In other embodiments, the antibodies are administered in an amount effective in reducing the activity level of a target antigen (e.g., Dectin-1, Galectin-9, or phosphorylation of Syk) by at least 20% (e.g., 30%, 40%, 50%, 60%, 70%, 80%, 90% or greater).

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., those encoding the antibodies described herein) are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

The particular dosage regimen, i.e., dose, timing and repetition, used in the method described herein will depend on the particular subject and that subject's medical history.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents.

Treatment efficacy for a target disease/disorder can be assessed by methods well-known in the art.

### (iv) Combined Therapy

Any of the Dectin-1 antagonists described herein may be utilized in conjunction with other types of therapy for cancer, such as chemotherapy, surgery, radiation, gene therapy, and so forth. Such therapies can be administered simultaneously or sequentially (in any order) with the immunotherapy according to the present disclosure.

When co-administered with an additional therapeutic agent, suitable therapeutically effective dosages for each agent may be lowered due to the additive action or synergy.

In some embodiments, the Dectin-1 antagonist can be combined with other immunomodulatory treatments such as, e.g., inhibitors of a checkpoint molecule (e.g., PD-1, PD-L1, PD-L2, CDLA-4, LAG3, TIM-3, or A2aR), activators of a co-stimulatory receptor (e.g., DX40, GITR, CD137, CD40, CD27, and ICOS), inhibitors of an innate immune cell target (e.g., KIR, NKG2A, CD96, TLR, and IDO). Without being bound by theory, it is reported herein that Dectin-1 antagonist can reprogram immune responses against tumor cells via, e.g., inhibiting the activity of γδ T cells infiltrated into tumor microenvironment, and/or enhancing immune surveillance against tumor cells by, e.g., activating CD4+ and/or CD8+ T cells. Thus, combined use of a Dectin-1 antagonist and an immunomodulatory agent such as those described herein would be expected to significantly enhance anti-tumor efficacy.

In other embodiments, the Dectin-1 antagonist described herein can also be co-used with a chemotherapeutic agent, including alkylating agents, anthracyclines, cytoskeletal disruptors (Taxanes), epothilones, histone deacetylase inhibitors, inhibitors of topoisomerase I, inhibitors of topoisomerase IL, kinase inhibitors, nucleotide analogs and precursor analogs, peptide antibiotics, platinum-based agents, retinoids, vinca alkaloids and derivatives thereof. Non-limiting examples include: (i) anti-angiogenic agents (e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000)); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones, and navelbine, epidipodophyllotoxins (etoposide and teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycin, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, mitoxantrone, topotecan, and irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; and chromatin disruptors.

Additional useful agents can be found in, e.g., Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

It was reported that chemotherapy and/or immune therapy of solid tumor could enhance the level of immune modulators such as checkpoint molecules, resulting in suppressed immunity against tumor cells. Erisson et al., J. Translational Medicine (2016), 14:282; Grabosch et al., J. ImmunoTherapy of Cancer (2015), 3(suppl 2): P302; and Azad et al., EMBO J. (2016). Here, Dectin-1 antagonists were found to reprogram immune responses targeting tumor cells, particularly in PDA. As such, the co-use of a Dectin-1 and a chemotherapeutic agent (e.g., gemcitabine) or immunotherapeutic agent (e.g., anti-PD-L1 antibody) would be expected to result in significantly enhanced therapeutic activity against solid tumors such as PDA.

### Kits for Use in Treating Solid Tumor

The present disclosure also provides kits for use in treating or alleviating a solid tumor such as PDA and CRC. Such kits can include one or more containers comprising a Dectin-1 antagonist, e.g., any of those described herein, and optionally a second therapeutic agent to be co-used with the Dectin-1 antagonist, which is also described herein.

In some embodiments, the kit can comprise instructions for use in accordance with any of the methods described herein. The included instructions can comprise a description of administration of the Dectin-1 antagonist, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of a Dectin-1 antagonist generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a solid tumor such as PDA or CRC. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a Dectin-1 antagonist as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### Methods for Diagnosing Solid Tumors

Also described herein are methods for determining the presence and/or measuring the level of Dectin-1 in a biological sample obtained from a subject who is suspected of having a solid tumor, for example, PDA. The Dectin-1 level thus determined may be used as a biomarker for assessing whether the subject has or is at risk for the solid tumor, or for assessing treatment efficacy of a treatment against the solid tumor on that subject.

Such an assay method can comprise at least the following steps: (i) obtaining a biological sample from a subject (e.g., a human patient) suspected of having solid tumor such as PDA; and (ii) measuring the level of Dectin-1 in the biological sample. The method may further comprise identifying the subject as having or at risk for the solid tumor if the Dectin-1 level thus measured is higher than the Dectin-1 level of a control subject (e.g., a solid tumor-free subject of the same species such as a PDA-free subject of the same species). A therapy for solid tumor such as PDA, e.g., those described herein or known in the art, can then be applied to the subject, if the subject is identified as having or at risk for the solid tumor such as PDA.

A subject suspected of having a solid tumor such as PDA may exhibit one or more symptoms associated with the solid tumor, for example, jaundice and related symptoms, dark urine, light-colored or greasy stools, itchy skin, belly or back pain, weight loss and poor appetite, nausea and vomiting, gallbladder or liver enlargement, and/or blood clots. Such a subject (e.g., a human patient) may be identified by routine medical procedures.

A suitable biological sample can be obtained from a subject as described herein via routine practice. Non-limiting examples of biological samples include fluid samples such as blood (e.g., whole blood, plasma, or serum), urine, and saliva, and solid samples such as tissue (e.g., skin, lung, nasal) and feces. Such samples may be collecting using any method known in the art or described herein, e.g., buccal swab, nasal swab, venipuncture, biopsy, urine collection, or stool collection. In some embodiments, the biological sample is a blood sample comprising one or more populations of immune cells. In other embodiments, the biological sample may be a tissue biopsy sample, which may be obtained from a suspected tumor site from the subject.

For prognosis purposes, any of the exemplary samples as described herein (e.g., blood samples or tissue samples) can be obtained from a subject prior to a treatment of a solid tumor (e.g., PDA), after the treatment, and/or during the course of the treatment.

In some embodiments, the sample may be processed or stored. Exemplary processing includes, for example, cell lysis and extraction of materials from the lysate (e.g., DNA, RNA, or protein). Exemplary storage includes, e.g., adding preservatives to the sample and/or freezing the sample.

The level of Dectin-1 in a biological sample can be represented by the level of Dectin-1 protein in the sample, the level of Dectin-1 mRNA in the sample, or the activity level of the Dectin-1 protein, or a combination thereof. Assays for measuring levels of mRNA, protein and Dectin-1 activity are known in the art and described herein, e.g., including probe-based assays, array-based assays, PCR-based assays, bead-based assays, immuno-based assays, sequencing, bisulfate assays, etc. (see, e.g., Molecular Cloning: A Laboratory Manual, J. Sambrook, et al., eds., Fourth Edition, Cold Spring Harbor Laboratory Press, Cold Spring Harbor, N.Y., 2012; Current Protocols in Molecular Biology, John Wiley & Sons, Inc., New York; Current Protocols in Gene Expression, John Wiley & Sons, Inc., New York; Microarray Methods and Protocols, R. Matson, CRC Press, 2012; Antibodies: A Laboratory Manual, Cold Spring Harbor Laboratory Press, 2^{nd} ed., 2013).

In some examples, the level of Dectin-1 protein in a biological sample (e.g., cellular, membrane, or both), such as a blood sample of a tissue sample, is measured via a suitable method. Exemplary protein level assays include, but are not limited to, immunoassays (e.g., Western blot or enzyme-linked immunosorbent assay (ELISA)) and multiplex bead-based assays. Such assays are known in the art and commercially available. In some examples, the cell-surface expression level of Dectin-1 is measured using a suitable method known in the art or described herein. Such assays may involve the use of a suitable antibody specific to Dectin-1, e.g., those described herein.

In other examples, a level of Dectin-1 mRNA is determined in a conventional method or a method described herein. Exemplary mRNA level assays include, but are not limited to probe-based assays (e.g., northern blots, nuclease protection assays, in situ hybridization), array-based assays (e.g., microarrays), PCR-based assays (e.g., quantitative PCR), multiplex bead-based assays (e.g., commercially-available Luminex® technology such as xMAP® and xTAG®, Illumina), and sequencing-based assays. Such assays are known in the art and commercially available.

In some examples, the activity level of Dectin-1 protein in a biological sample is measured via a suitable method. Exemplary activity level assays include assays for measuring activation of one or more of the downstream components in the Dectin-1 pathway, for example, phosphorylation of Syk.

In a further example, the level of nuclear Dectin-1 of a sample can be assessed using the Histo (H)-score approach, which is a method known in the art to assess the extent of nuclear immunoreactivity. Briefly, the staining intensity (0, 1+, 2+, or 3+) is determined for each cell in a fixed field. The percentage of cells at each staining intensity level is calculated, and an H-score is assigned using the following formula:

[1×(% cells1+)+2×(% cells2+)+3×(% cells3+)]

Therefore, the H-score can range from 0-300. A program, such as X-tile, is then used to establish cutoffs within the calculated range of the data. For example, H score cutoffs that correlate with survival can be determined, which can then be validated in a validation data set.

In another example, the level of Dectin-1, particularly the level of Dectin-1 in cellular membranes and/or cytoplasm, in a fixed field can be assessed using the intensity score method, which is also well developed in the art.

The Dectin-1 level of a biological sample obtained from a subject as described herein can be relied on to determine whether the subject has or at risk for the solid tumor such as PDA. If the subject is a patient having a solid tumor and is under a treatment of the solid tumor, the change of Dectin-1 levels before and after the treatment, or during the course of the treatment, could be relied on to evaluate the treatment efficacy on that subject. In some examples, the Dectin-1 level of the candidate subject can be compared with a pre-determined value as described herein, or the Dectin-1 level of a control subject, which can be a subject of the same species and free of the solid tumor such as PDA. Optionally, the control subject has matched age, gender, and other physical features as the candidate subject. An elevated level of Dectin-1 in the biological sample as compared with the pre-determined value or the Dectin-1 level of the control subject indicates that the subject has or at risk for the solid tumor such as PDA. Alternatively, a decrease of Dectin-1 in a subject undergoing an anti-tumor treatment (e.g., anti-PDA treatment) after the treatment of along the course of the treatment is indicative of treatment efficacy.

As used herein, “an elevated level of Dectin-1” means that the level of Dectin-1 is above a pre-determined value, such as a pre-determined threshold or the level of Dectin-1 in a control subject as described herein, e.g., at least 20%, 30%, 40%, 50%, 60%, 70%, 80%, 90%, 100%, 2-fold, 5-fold, 10-fold, 50-fold, or 100-fold higher than the pre-determined value or the level of the control subject. An elevated level of Dectin-1 also includes increasing a phenomenon from a zero state (e.g., no or undetectable Dectin-1 in a control) to a non-zero state (e.g., some Dectin-1 or detectable Dectin-1 in a sample).

A pre-determined value can be the Dectin-1 level in a control sample (a controlled level), which can be measured using any of the methods known in the art or described herein. In some examples, the pre-determined value is measured by the same method applied for measuring the Dectin-1 level in a biological sample. The control level may be a level of the Dectin-1 in a control sample, control subject, or a population of control subjects.

The control may be (or may be derived from) a normal subject (or normal subjects). Normal subjects, as used herein, refer to subjects that are apparently healthy and show no signs or symptoms of a solid tumor such as PDA (free of the solid tumor). The population of control subjects may therefore be a population of normal subjects.

It is to be understood that the methods provided herein do not require that a control level be measured every time a subject is tested. Rather, in some embodiments, it is contemplated that control levels are obtained and recorded and that any test level is compared to such a pre-determined level. The pre-determined level may be a single-cutoff value or a range of values.

By comparing the Dectin-1 level(s) of one or more biological samples obtained from a subject and the pre-determined value as described herein, the subject can be identified as having or at risk for the solid tumor such as PDA. Further, decrease of Dectin-1 during a course of treatment is indicative that the treatment is effective on the subject.

A subject identified by any of the diagnostic methods described herein may be treated by a conventional anti-tumor therapy (e.g., anti-PDA therapy) or any of the treatment methods described herein.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Dectin-1 Signaling Drives Pancreatic Oncogenesis by Promoting Adaptive Immune Suppression

Pancreatic ductal adenocarcinoma (PDA) is a devastating disease in which the mortality rate approaches the incidence rate (Yadav et al., Gastroenterology, 2013, 144, 1252-1261). Specifically, PDA is almost invariably associated with a robust inflammatory infiltrate which can have divergent influences on disease progression by either combating cancer growth via antigen-restricted tumoricidal immune responses or by promoting tumor progression via induction of immune suppression (Zheng et al., Gastroenterology, 2013, 144, 1230-1240; Clark et al., Cancer Res., 2007, 67, 9518-9527; Andren-Sandberg et al., Scand J Gastroenterol., 1997, 32, 97-103). For example, CD8⁺ T cells and Th1-polarized CD4⁺ T cells mediate tumor protection in murine models of PDA and are associated with prolonged survival in human disease (Fukunaga et al., Pancreas, 2004, 28, e26-e31). Conversely, it has been reported that Th2-polarized CD4⁺ T cells promote PDA progression in mice and intra-tumoral CD4⁺ Th2 cell infiltrates correlate with reduced survival in human disease (Ochi et al., J Exp Med., 2012, 207, 1671-1687; Fukunaga et al., Pancreas, 2004, 28, e26-e31; De Monte et al., J Exp Med 2011, 208, 469-478). Similarly, Foxp3⁺ Tregs facilitate tumor immune escape and shorten survival in PDA (Hiraoka et al., Clin Cancer Res., 2006, 12, 5423-5434; Jiang et al., PloS One, 2014, 9, e106741). Hence, T cell programing influences disease outcome in PDA. However, regulation of the balance between immunogenic and immune-suppressive T cell populations is uncertain. Data of the present disclosure suggest that Dectin-1 signaling plays a critical role in the capacity of macrophages to educate CD4⁺ and CD8⁺ T cells toward immunogenic or tolerogenic phenotypes.

### Methods

### Animals and In Vivo Models

C57BL/6 (H-2Kb) mice were purchased from Jackson Labs (Bar Harbor, Me.) and bred in-house. KC and KPC mice develop pancreatic neoplasia endogenously by expressing mutant Kras alone or mutant Kras and p53, respectively, in the progenitor cells of the pancreas (Hingorani et al., Cancer Cell, 2003, 4, 437-450; Hingorani et al., Cancer Cell, 2005, 7, 469-483). Tumor progression and survival in control KC mice has been previously detailed (Daley et al., Cell, 2016, 166, 1485-1499). Dectin-1^{-/-} mice were crossed with KC mice to generate KC; Dectin-1^{-/-} animals. For orthotopic pancreatic tumor challenge, mice were administered intra-pancreatic injections of either Kras^{G12D} PDEC or FC1242 tumor cells derived from KPC mice. Kras^{G12D} PDEC and FC1242 cells were generated as previously described (Pylayeva-Gupta et al., Cancer Cell, 2012, 21, 836-847; Zambirinis et al., J Exp Med., 2015, 212, 2077-2094). In select experiments, KPC-derived tumor cells (1×10⁶) engineered to express OVA using pCI-neo-cOVA (Addgene plasmid #25097) were used as previously described (Daley et al., Cell, 2016, 166, 1485-1499). Both male and female mice were used but animals were sex- and age-matched in each experiment. For orthotopic tumor experiments, 8-10 week old mice were used. In preparation for intra-pancreatic injection, cells were suspended in PBS with 50% Matrigel (BD Biosciences, Franklin Lakes, N.J.) and 1×10⁵ tumor cells were injected into the body of the pancreas via laparotomy. Mice were sacrificed 3 weeks later and tumor weight recorded. In select experiments, KPC-derived tumor cells (5-10×10⁵) were administered subcutaneously alone or mixed with macrophages (2×10⁵). To study the effects of Dectin-1 ligation, mice were administered d-Zymosan (500 ug) or HKCA (5×10⁷ cells; both Invivogen, San Diego, Calif.) by intraperitoneal (i.p.) injection five times weekly for 8 weeks in endogenous tumor models and for 3 weeks in the orthotopic tumor models. PDEC were harvested from pancreata of KC mice and passaged in vitro as previously described (Pylayeva-Gupta et al., Cancer Cell, 2012, 21, 836-847). PDEC proliferation was measured using the XTT assay according to the manufacturer's protocol (Roche, Nutley, N.J.). In select experiments, cohorts of mice were treated five times weekly with the p-Syk inhibitor Piceatannol (20 mg/kg, i.p.; Selleck Chemicals, Houston, Tex.). Pan-T cells (CD90, T24/31), CD4 T cells (GK1.5), CD8 T cells (53-6.72), and macrophages (F4/80, CI:A3-1, all BioXcell, West Lebanon, N.H.) were depleted with neutralizing mAbs using regimens previously described (Seifert et al., Nature, 2016, 532, 245-249; Bedrosian et al., Gastroenterol., 2016, 141, 1915-1926, e1911-1914). In other experiments, animals were treated twice weekly with i.p. injection of neutralizing mAbs directed against PD-1 (29F.1A12, 6 mg/kg), or Galectin-9 (RG9-1, 6 mg/kg; both BioXCell) or respective isotype controls. Bone marrow chimeric animals were created by irradiating mice (9 Gy) followed by i.v. bone marrow transfer (lx 10⁷ cells) from non-irradiated donors as previously described (Ochi et al., J Clin Invest., 2012, 122, 4118-4129). Chimeric mice were used in experiments 7 weeks later. All animal procedures were approved by the New York University School of Medicine IACUC.

### Cellular Harvest and Flow Cytometry

Human or murine single cell suspensions for flow cytometry were prepared as described previously with slight modifications (Seifert et al., Nature, 2016, 532, 245-249). Briefly, pancreata were placed in cold RPMI 1640 with Collagenase IV (1 mg/mL; Worthington Biochemical, Lakewood, N.J.) and DNAse I (2 U/mL; Promega, Madison, Wis.) and minced with scissors to sub-millimeter pieces. Tissues were then incubated at 37° C. for 30 minutes with gentle shaking every 5 minutes. Specimens were passed through a 70 μm mesh, and centrifuged at 350 g for 5 minutes. The cell pellet was resuspended in cold PBS with 1% FBS. Single cell splenocyte suspensions were prepared as previously described (Seifert et al., Nature, 2016, 532, 245-249). Cell labeling was performed after blocking FcγRIII/II with an anti-CD16/CD32 mAb (eBioscience, San Diego, Calif.) by incubating 1×10⁶ cells with 1 μg of fluorescently conjugated mAbs directed against murine CD44 (IM7), CD206 (C068C2), PD-1 (29F.1A12), CD3 (17A2), CD4 (RM4-5), CD8 (53-6.7), CD45 (30-F11), CD11b (M1/70), Gr1 (RB6-8C5), CD11c (N418), MHC II (M5/114.15.2), IL-6 (MP5-20F3), IL-5 (TRFK5), IL-10 (JES5-16E3), IFN-γ (XMG1.2), TNFα (MP6-XT22), F4/80 (BM8), ICOS (15F9), OX40 (OX86), CD133 (315-2c11), CD62L (MEL-14), CD107a (1D4B), Galectin-9 (RG9-35; all Biolegend, San Diego, Calif.), Thet (eBio4B10), iNOS (CXNFT), IL-13 (eBio13A), Granzyme B (NGZB), FoxP3 (FJK-16s), p-Syk (moch1ct; all eBioscience), Dectin-1 (2A11, Abcam), and Dectin-1 Fc (fc-mdec1a; InvivoGen). Human PDA-infiltrating cells and PBMC were stained with mAbs directed against CD45 (HI30), CD14 (HCD14), CD15 (W6D3), Galectin-9 (9M1-3), and CD11c (3.9; all Biolegend). OVA-restricted CD8⁺ T cells were identified using an OVA-Pentamer (Proimmune, Oxford, UK). Dead cells were excluded from analysis using zombie yellow (Biolegend). Intracellular cytokine staining was performed using the Fixation/Permeabilization Solution Kit (BD Biosciences) for cytokines, transcription factors, and Granzyme B. Flow cytometry was carried out on the LSR-II flow cytometer (BD Biosciences). Data were analyzed using FlowJo v.10.1 (Treestar, Ashland, Oreg.). BMDM were prepared as previously described (Greco et al., J Leukocyte Biol., 2016, 100, 185-194). In select experiments, IL-12 (0.1 ng/ml; both R&D Systems, Minneapolis, Minn.), TNF-α (8 μg/ml; Cell Signaling, Beverly, Mass.), or TGF-β (0.2 ng/ml; Biolegend) were added to day 8 BMDM cultures for 24h. Alternatively, BMDM were cocultured with KPC-derived tumor cells (50:1 ratio).

### Histology, Immunohistochemistry, and Microscopy

For histological analysis, pancreatic specimens were fixed with 10% buffered formalin, dehydrated in ethanol, embedded with paraffin, and stained with H&E or Gomori's Trichrome. The fraction of preserved acinar area was calculated as previously described (Seifert et al., Nature, 2016, 532, 245-249). Pancreatic ductal dysplasia was graded according to established criteria (Hruban et al., Am J Surg Pathol., 2001, 25, 579-586). Immunohistochemistry on frozen or paraffin embedded mouse tissues was performed using antibodies directed against F4/80 (CI:A3-1, Conc: 10 μg/ml), Arginasel (Polyclonal, Conc: 2 μg/ml), p-Syk (Polyclonal, Conc: 10 μg/ml), Ki67 (Polyclonal, Conc: 3 μg/ml), and Dectin-1 (2A11, Conc: 5 μg/ml, all Abcam). For paraffin-embedded samples (F4/80, p-Syk, Ki67, Arginasel), samples were dewaxed in ethanol followed by antigen retrieval with 0.01M Sodium Citrate with 0.05% Tween. For frozen specimen (Dectin-1), samples did not undergo antigen retrieval prior to incubation with the primary antibody. Immunofluorescent staining on frozen mouse tissues was performed using antibodies against Dectin-1 (2A11; Conc: 20 μg/ml, Abcam), CD45 (30-F11; Conc: 6.25 μg/ml, BD Biosciences), CK19 (Troma-III; University of Iowa), CD68 (FA-11, Conc: 5 μg/ml, Abcam), Dectin-1 Fc (fc-mdec1a; InvivoGen), Galectin-9 (Polyclonal; Conc: 20 μg/ml, Bioss) and DAPI (Vector Labs, Burlingame, Calif.). Immunofluorescent staining in human tissue was performed using antibodies against Dectin-1 (Polyclonal; Conc: 20 μg/ml, Abcam), CD11b (M1/70, Cone: 5 μg/ml), Ep-CAM (G8.8; Conc: 5 μg/ml, both Biolegend) and DAPI (Vector Labs, Burlingame, Calif.). Immunofluorescent images were acquired using the Zeiss LSM700 confocal microscope with ZEN 2010 software (Carl Zeiss, Thornwood, N.Y.). All human tissues were collected using an IRB approved protocol and donors of de-identified specimens gave informed consent. Quantifications were performed by assessing 10 high-power fields (HPF; 40×) per slide in a blinded manner.

### Western Blotting and RNA Analysis

For protein extraction, tissues were homogenized in ice-cold RIPA buffer. Total protein was quantified using the DC Protein Assay according to the manufacturer's instructions (BioRad, Hercules, Calif.). Western blotting was performed as previously described with minor modifications (Seifert et al., Nature, 2016, 532, 245-249). Briefly, 10% Bis-Tris polyacrylamide gels (NuPage, Invitrogen) were equiloaded with 10-30 μg of protein, electrophoresed at 200V, and electrotransferred to PVDF membranes. After blocking with 5% BSA, membranes were probed with primary antibodies to β-actin (8H10D10), p53 (7F5), PLC-γ (polyclonal), p-PLC-γ (polyclonal), Bcl-XL (54H6; all Cell Signaling), JNK (2C6), p-JNK (G9), Smad4 (polyclonal), p16 (polyclonal), c-Myc (9E10), CARD9 (polyclonal), Syk (polyclonal), p-Syk (polyclonal), Rb (C-15; all Cell Signalling), Dectin-1 (polyclonal; Abcam), Galectin-9 (polyclonal), and Dectin-1 Fc (fc-mdec1a; InvivoGen). Blots were developed by ECL (Thermo Scientific, Asheville, N.C.). RNA extraction was performed using the RNeasy Mini kit (Qiagen, Germantown, Md.) as per manufacturer's instructions. For Nanostring analysis, the nCounter mouse inflammation panel was employed using the nCounter Analysis System (both Nanostring, Seattle, Wash.).

### Enrichment of Endogenous Dectin-1 Ligands and Mass Spectroscopy

Cell lysate of KPC-derived tumor cells were prepared and protein was quantified as above. Lysate (2 mg) was mixed overnight at 4° C. with a human IgG Fc-conjugated Dectin-1 fusion protein (3 mg) and protein-G magnet beads (10 ml; 10003D, Dynabeads, FisherThermo, Grand Island, N.Y.). Magnet beads were then washed once with 1% NP40 lysis buffer, three times with 0.5M NaCl, and once with H₂O. The affinity purified sample was eluted off the beads by boiling with SDS loading buffer. Samples were reduced with DTT at 57° C. for 1 hour and then alkylated with iodoacetamide at 37° C. in the dark for 45 minutes (2 μl of 0.5M in 100 mM ammonium bicarbonate). After alkylation, samples were loaded onto a NuPAGE 4-12% Bis-Tris Gel 1.0 mm (Life Technologies Corporation, Grand Island, N.Y.) and run for 15 minutes at 200 V. The gel was stained using GelCode Blue Stain Reagent (Thermo Scientific, Rockford, Ill.). The short gel lane was cut into approximately 1 mm³ pieces. The gel pieces were destained in 1:1 v/v solution of methanol and 100 mM ammonium bicarbonate at 4° C. with agitation. The destain solution was changed every 15 minutes at least 5 times and until pieces had no visibly blue stain left. Gel pieces were partially dehydrated with an acetonitrile rinse and further dried in a SpeedVac concentrator for 20 minutes. Sequencing grade-modified trypsin (300 ng; Promega, Madison, Wis.) was added to the dried gel pieces. After the trypsin was absorbed, 200 μl of 100 mM ammonium bicarbonate was added to cover the gel pieces and digestion proceeded overnight on a shaker at 37° C. Peptide extraction was performed by adding a slurry of R2 20 μm POROS® beads (Life Technologies Corporation) in 5% formic acid; 0.2% trifluoroacetic acid (TFA) to each sample at an volume equal to that of the ammonium bicarbonate. Samples were incubated with agitation at 4° C. for 4 hours. The beads were loaded onto equilibrated C18 ZIPTIPS® (Millipore) using a microcentrifuge for 30 sec at 6000 RPM. Gel pieces were rinsed three times with 0.1% TFA and each rinse was added to the corresponding ZIPTIP® followed by microcentrifugation. Extracted POROS® beads were further washed with 0.5% acetic acid. Peptides were eluted off the beads by addition of 40% acetonitrile in 0.5% acetic acid followed by the addition of 80% acetonitrile in 0.5% acetic acid. The organic solvent was removed using a SpeedVac concentrator and the samples were reconstituted in 0.5% acetic acid. An aliquot of each sample was loaded onto the EASY spray 50 cm C18 analytical HPLC column with <2 μm bead size using the auto sampler of an EASY-nLC 1000 HPLC (ThermoFisher) in solvent A (2% acetonitrile, 0.5% acetic acid). The peptides were gradient eluted directly into a Q Exactive (Thermo Scientific) mass spectrometer using a one hour gradient from 2% to 31% solvent B (95% acetonitrile, 0.5% acetic acid), followed by 10 minutes from 31% to 40% solvent B, and 10 minutes from 40% to 100% solvent B. The Q Exactive mass spectrometer acquired high resolution full MS spectra with a resolution of 70,000, an AGC target of 1×10⁶, with a maximum ion time of 120 ms, and scan range of 400 to 1500 m/z. HCD MS/MS spectra were acquired using the following instrument parameters: resolution of 17,500, AGC target of 5×10⁴, maximum ion time of 120 ms, one microscan, 2 m/z isolation window, fixed first mass of 150 m/z, and Normalized Collision Energy of 27, dynamic exclusion of 30 seconds. The MS/MS spectra were searched against the Uniprot Mouse database combined with mammalian IgG database using Sequest within Proteome Discoverer (ThermoFisher). The results were filtered using a<1% False Discovery Rate searched against a decoy database and all the proteins with less than two unique peptides were excluded. Proteins identified in the control were subtracted from the proteins identified in the Dectin-1 affinity purification and a shortened list interrogated for potential Dectin-1 ligands.

### Analysis of Dectin-1-Galectin-9 Interaction

To investigate if Galectin-9 can bind with Dectin-1, Protein G-magnet beads (2 ml; Dynabeads) were loaded with Dectin-1 IgG Fc (1 mg) or control IgG Fc before washing and blocking. Subsequently, beads were incubated with recombinant Galectin-9 (2 mg; R&D Systems) for 30 min, washed, and stained with PE-conjugated anti-Galectin-9 (Biolegend, San Diego Calif.). Galectin-9 specific staining was determined by flow cytometry. To investigate the interaction between Galectin-9 and Dectin-1 by ELISA, plates (Maxisorp, Nunc, St. Louis, Mo.) were coated with recombinant mouse Galectin-9 (2 mg; R&D Systems, Minneapolis, Minn.), Galectin-3, or Galectin-4 (both 2 mg; Biolegend) for 16 hours at 4° C., blocked with 1% BSA/PBS for 1 hour, and incubated with increasing doses of Dectin-1 IgG Fc or control IgG Fc for 2 hours. The Galectin-bound Dectin-1 IgG Fc was detected with anti-IgG-HRP. For affinity precipitation experiments, protein G-beads were loaded with Dectin-1 IgG Fc and were then incubated with the extract of 6 month old KC pancreata. Bead-bound precipitates were resuspended with loading buffer, resolved by SDS-PAGE under reduced conditions for western blotting using mAbs specific for Galectin-9. To determine whether Galectin-9 induces Dectin-1 signaling, the Dectin-1 reporter HEK293 cell line¹⁶ (Invivogen) was treated with recombinant Galectin-9 (1-10 μg/ml; R&D Systems), d-Zymosan (1-10 μg/ml), or Curdlan (10-100 μg/ml; both Invivogen). Dectin-1 signaling was measured by detection of secreted embryonic alkaline phosphatase. In other experiments, WT and Dectin-1^{-/-} splenic macrophages were treated in vitro with recombinant Galectin-9 (10 ug/ml). Syk phosphorylation was measured by flow cytometry at 3 hours.

Galectin-9 Immunoprecipitation Experiments with PNGase F or Lactulose Treatment

Dectin-1 IgG Fc (8 μg; InvivoGen) was treated with PNGase F (20 μl, ˜10,000 units; New England Biolabs) following the company's non-denaturing protocol (37° C., 24 hr). PNGase F treated and untreated Dectin-1 IgG Fc samples (4 μg; InvivoGen) were added to protein G beads (25 μl, Dynabeads Protein G; novex) and the mixtures were incubated at room temperature for 10 min. The beads were washed 2× with PBS-T (pH 7.4 with 0.02% tween). Recombinant mouse Galectin-9 (4 μg; R&D Systems) was resuspended in PBS-T or a solution of 100 mM lactose in PBS-T. Galectin-9 samples were then incubated with the Dynabead/Dectin-1 IgG Fc complex for 20 min at room temperature. The Dynabeads were then washed 3× with PBS-T and transferred to a clean tube. Galectin-9 and Dectin-1 IgG Fc were eluted in SDS-PAGE buffer (PBS with 10% BME) by heating at 98° C. for 10 min. The samples were then analyzed by SDS-PAGE and stained with Coomassie Blue.

### Dectin-1 Knockdown

Lentivirus was prepared by infecting 293T cells with the either a Scrambled or shDectin (NM_020008.1-298s1c1) plasmid, Δ8.9CR2 plasmid, and vesicular stomatitis virus glycoprotein plasmid (3:1:4 ratio). Supernatant were collected for 3 days post infection. KPC cells were then infected with supernatant in the presence of polybrene (8 μg/ml) for 12 hours×2 and selected with puromycin (2 μg/ml). The efficacy of gene knockdown was confirmed by PCR, flow cytometry, and western blotting.

### Statistical Analysis

Data is presented as mean+/−standard error. Human RNAseq data and clinical correlations were performed using the UCSC Cancer Genomics Browser (genomecancer.ucsc.edu) (Chiba et al., eLife, 2014, 3, e04177). Survival was measured according to the Kaplan-Meier method. Statistical significance was determined by the Student's t test (two-tailed) and the log-rank test using GraphPad Prism 7 (GraphPad Software, La Jolla, Calif.). P-values <0.05 were considered significant.

### Results

### High Dectin-1 Expression in Murine and Human PDA

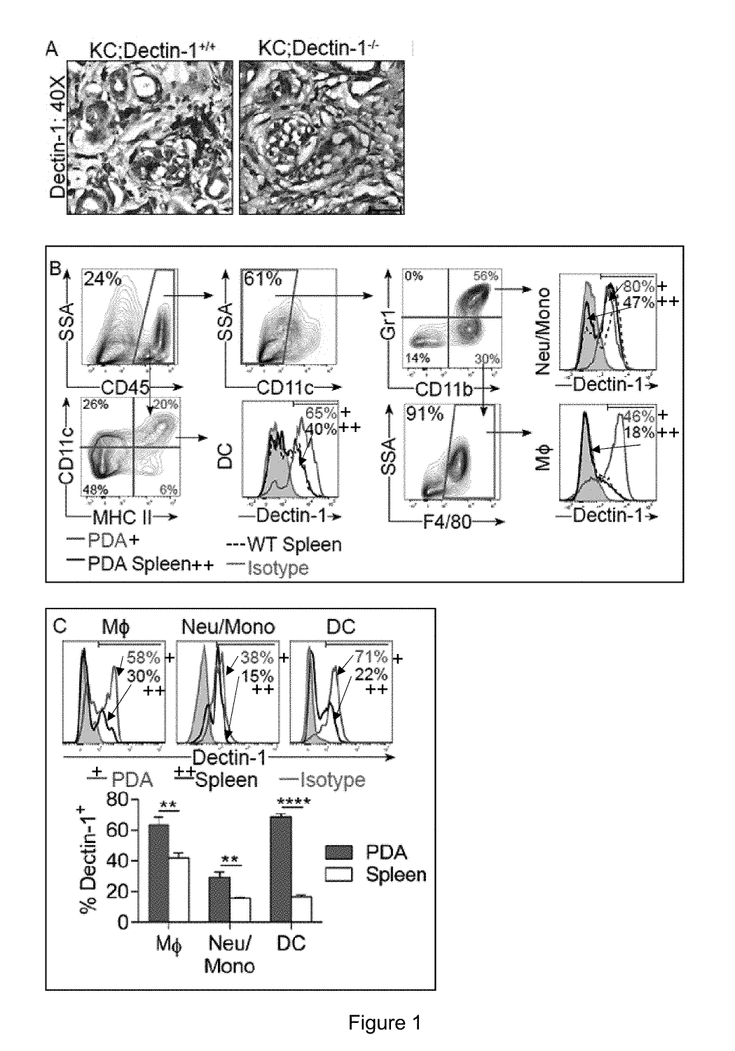

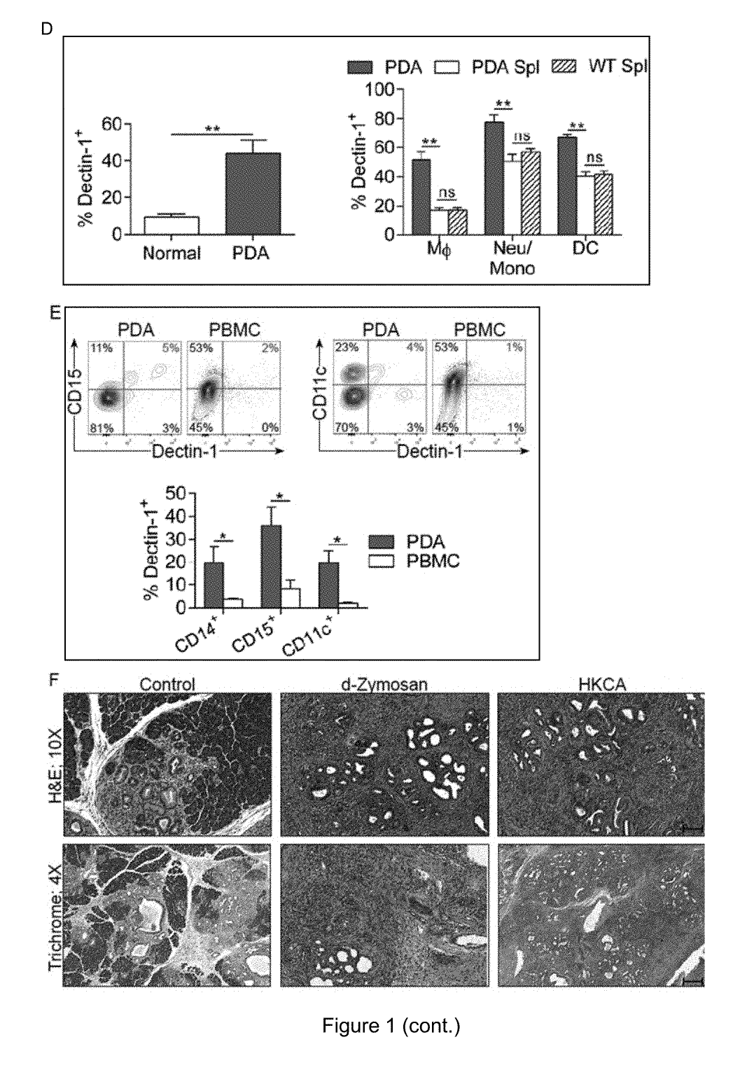

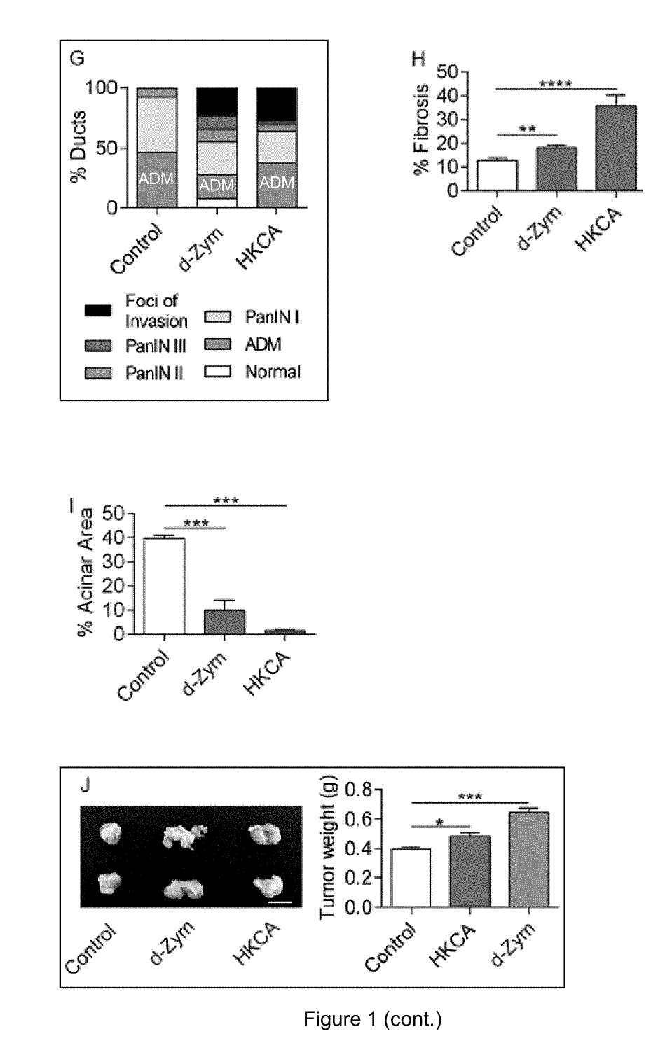

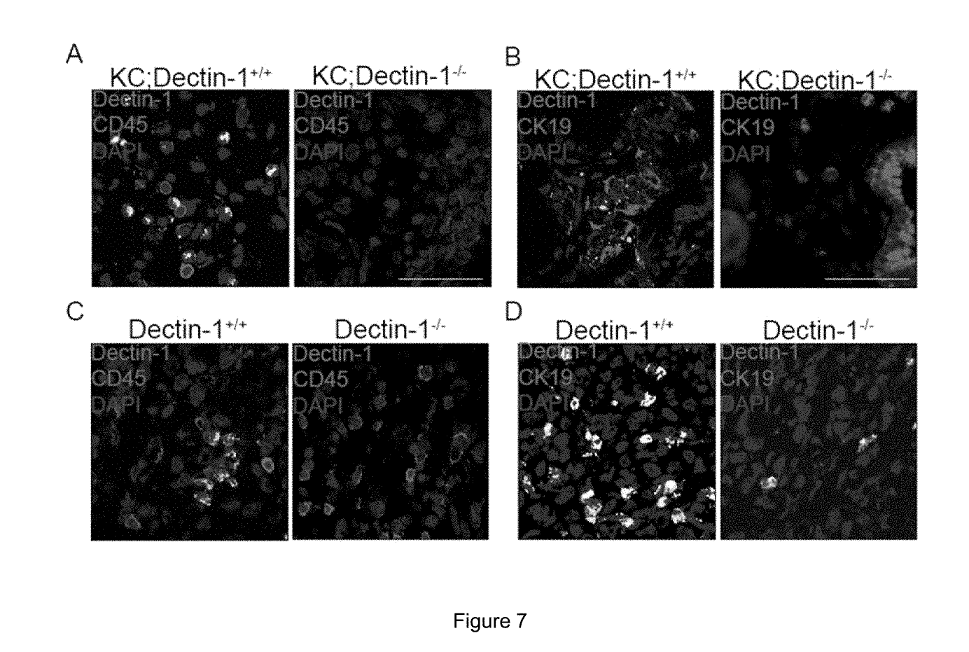

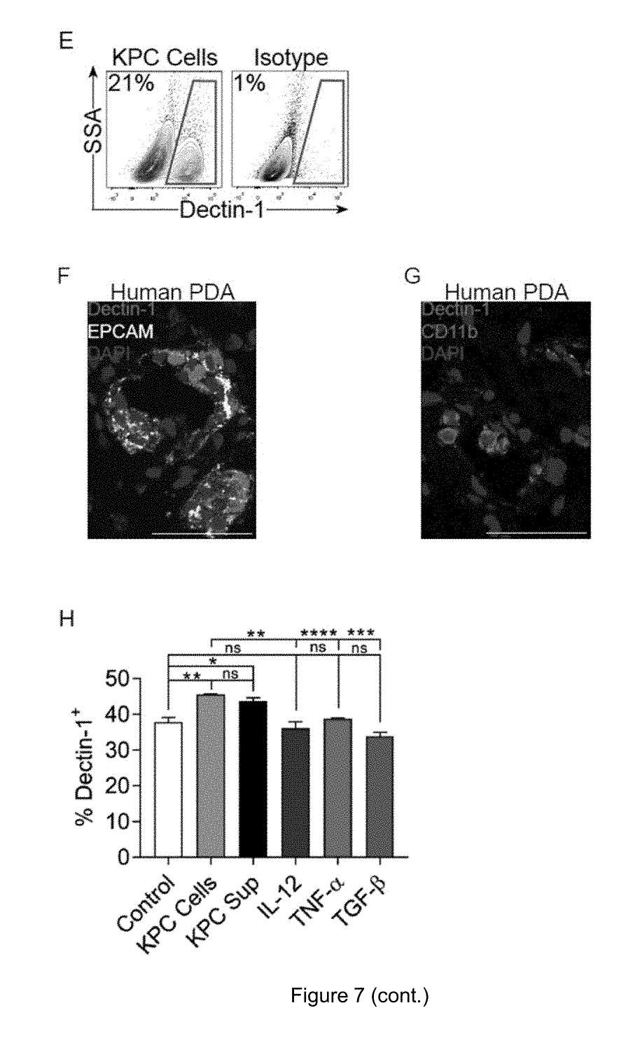

To test the relevance of Dectin-1 signaling to pancreatic ductal adenocarcinoma (PDA), Dectin-1 expression was examined in two PDA mouse models: the slowly progressive PDA model p48^{Cre}; LSL-Kras^{G12D} (KC) in which mice express oncogenic Kras in their pancreatic progenitor cells, and a more aggressive orthotopic PDA model utilizing tumor cells from Pdx1^{Cre}; LSL-Kras^{G12D}; Tp53^{R172H} (KPC) mice, which expresses mutant Kras and p53; as well as in human PDA (Hingorani et al., Cancer Cell, 2003, 4, 437-450; Hingorani et al., Cancer Cell, 2005, 7, 469-483). Immunohistochemical analysis suggested high Dectin-1 expression in leukocytes in KC pancreata and in transformed epithelial cells (FIG. 1, Panel A, and FIG. 7, Panels A and B). FIG. 1, Panel A shows frozen sections of 6 month-old KC; Dectin-1^{+/+} and KC; Dectin-1^{-/-} pancreata tested for expression of Dectin-1 by inmmunohistochemistry (IHC). FIG. 7, Panels A and B show frozen sections of 6 month-old KC; Dectin-1^{+/+} and KC; Dectin-1^{-/-} pancreata co-stained for Dectin-1 and CD45 (Panel A) or Dectin-1 and CK19 (Panel B) and imaged by confocal microscopy. Pancreata from KC mice crossed with Dectin-1^{-/-} animals (KC; Dectin-1^{-/-}) served as controls. Flow cytometry revealed ˜2-fold higher Dectin-1 expression in CD11c⁻Gr1⁻CD11b⁺F4/80⁺ macrophages (46%), Gr1⁺CD11b⁺ neutrophils and inflammatory monocytes (80%), and CD11c⁺MHCII⁺ dendritic cells (DC) (65%) within the KC TME compared with their cellular counterparts in spleen (18%, 47% and 40% respectively) (FIG. 1, Panel B). Similarly, in orthotopically implanted KPC tumors, Dectin-1 was highly expressed on leukocytes and in malignant epithelial cells (FIG. 1, Panel C and FIG. 7, Panels C to E). Dectin-1 was also expressed higher in leukocytes in PDA (44%) compared with normal pancreas (9%) (FIG. 1, Panel D). Immune-fluorescent microscopy in human PDA similarly suggested high Dectin-1 expression in transformed epithelial cells and in tumor-infiltrating myeloid cells (FIG. 7, Panels F and G). Parallel to mice, human PDA-infiltrating CD14⁺ and CD15⁺ monocytes and macrophages and CD11c DC expressed higher Dectin-1 compared with their cellular counterparts in peripheral blood mononuclear cells (PBMC) (FIG. 1, Panel E).

To investigate the stimulus for upregulation of Dectin-1 expression in leukocytes in PDA, bone-marrow derived macrophages (BMDM) were co-cultured with pancreatic tumor cells derived from KPC mice. PDA cells and PDA cellular supernatant upregulated Dectin-1 expression in BMDM; however, select cytokines (IL-12, TNF-α, and TGF-3) associated with PDA did not influence Dectin-1 expression (FIG. 7, Panel H). Collectively, these results suggest upregulated Dectin-1 expression in the tumor and peritumoral inflammatory compartment in PDA.

### Evidence of Dectin-1 Signaling and the Presence of Dectin-1 Ligands in PDA

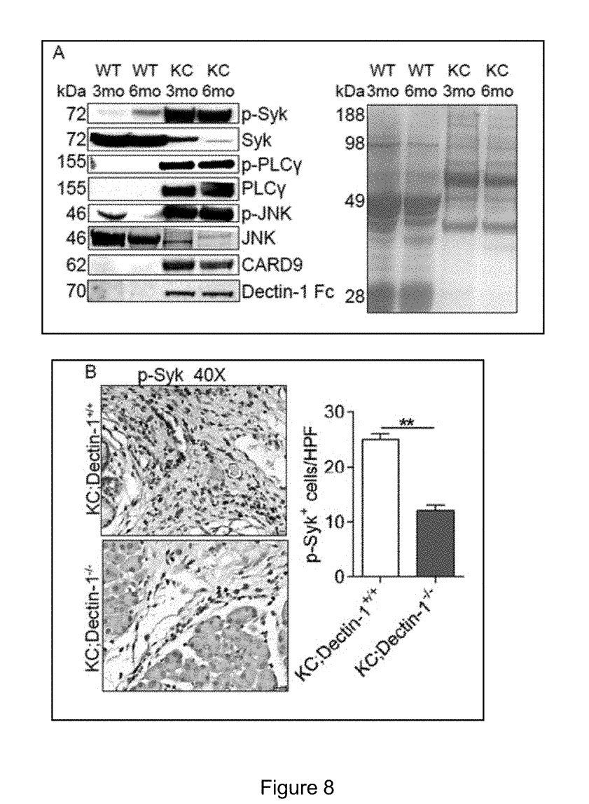

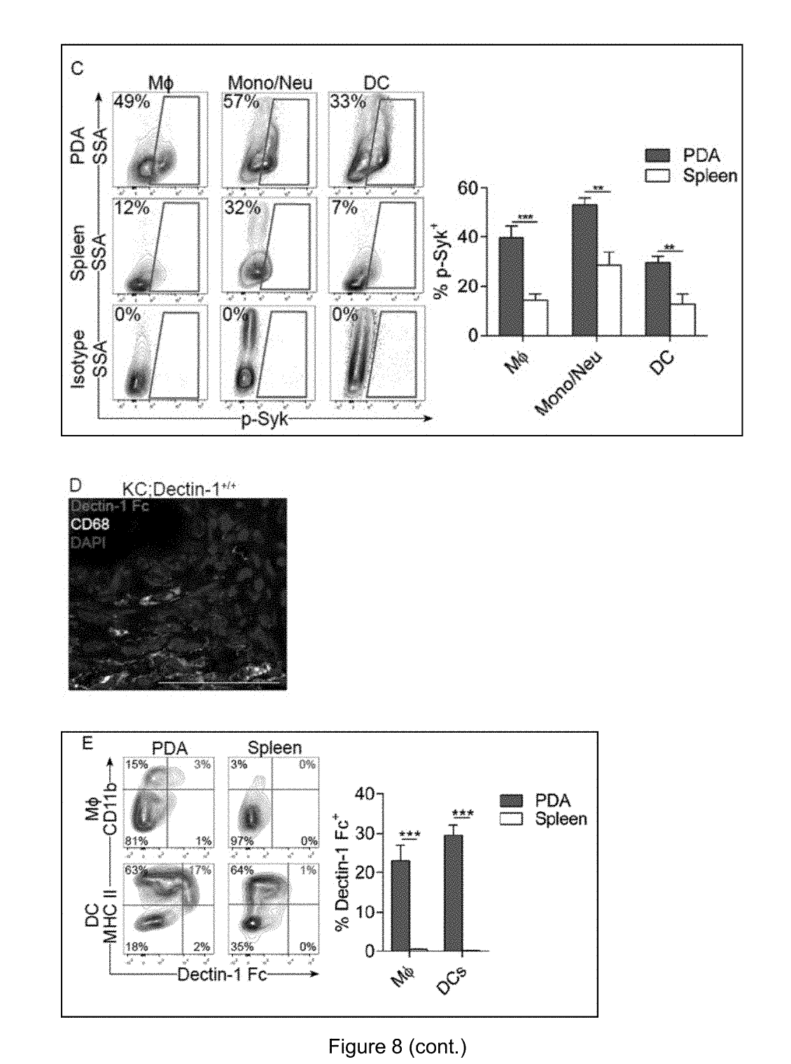

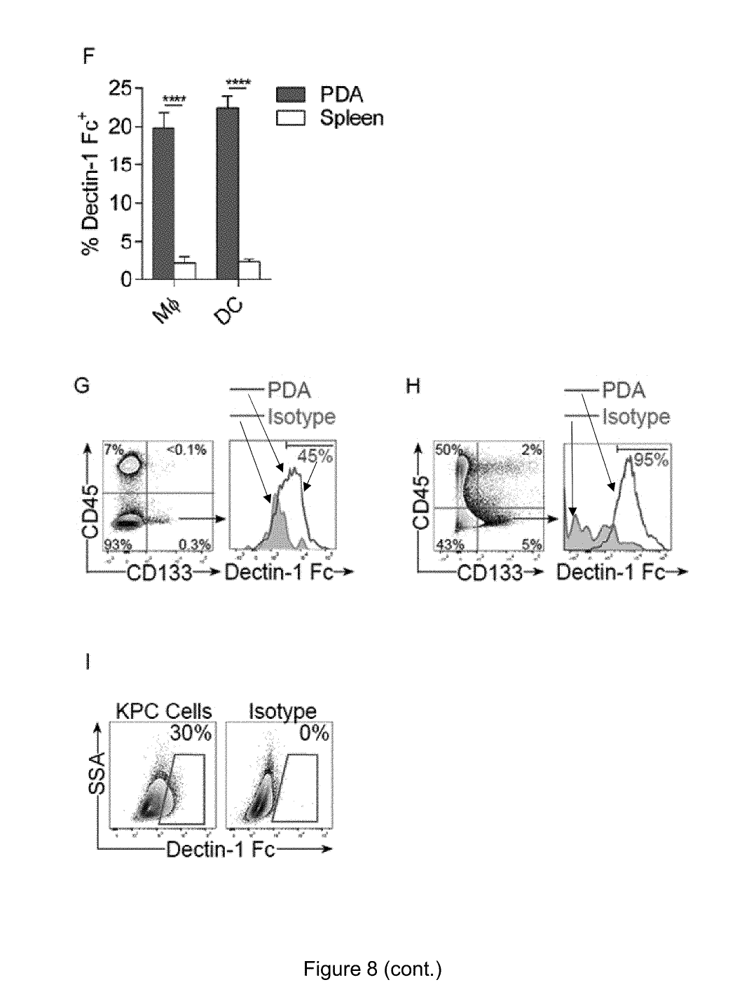

To investigate the relevance of Dectin-1 signaling in pancreatic oncogenesis, 3 and 6 month-old WT and KC pancreata lysates were assayed for the presence of activated signaling intermediates downstream of Dectin-1 ligation. In addition, intra-pancreatic Dectin-1 ligands were bound using a human IgG Fc conjugated Dectin-1 fusion protein and assayed for using α-human IgG. It was found that, compared with age-matched WT pancreata, KC pancreata expressed elevated p-Syk and p-PLCγ as well as high CARD9 and evidence of robust JNK pathway activation (FIG. 8, Panel A). Pancreata from 6 month-old KC and KC; Dectin-1^{-/-} mice were tested for expression of p-Syk by IHC, which confirmed high p-Syk expression in KC pancreata, whereas Syk signaling was reduced in PDA in the context of Dectin-1 deletion (FIG. 8, Panel B). Similarly, flow cytometry in CD11c⁻Gr1⁻CD11b⁺ macrophages, Gr1⁺CD11b⁺ neutrophils and inflammatory monocytes, and Gr1⁻CD11c⁺MHC II⁺DC from orthotopic KPC tumor or from the spleen of the same mice showed elevated p-Syk expression in diverse myeloid cellular subsets in PDA compared with their counterparts in the spleen (FIG. 8, Panel C).

To investigate for the presence of Dectin-1 ligands within the pancreatic TME, a human IgG Fc-conjugated Dectin-1 fusion protein was used. Whereas Dectin-1 ligands were absent in WT pancreata, high levels of Dectin-1 ligands were identified in pancreata of KC mice by Western blotting (FIG. 8, Panel A) and immune fluorescence microscopy (FIG. 8, Panel D). Flow cytometric analysis confirmed expression of Dectin-1 ligands on tumor-infiltrating macrophages and DC in KC pancreata compared with no expression in leukocytes in spleen, as the expression of Dectin-1 ligands in macrophages and DC in 6 month-old KC mice was tested by flow cytometry using the IgG Fc-conjugated Dectin-1 fusion protein and a fluorescently conjugated α-human IgG. (FIG. 8, Panel E). Expression of Dectin-1 ligands was similarly elevated in APCs infiltrating orthotopic KPC tumors (FIG. 8, Panel F). Further, CD133⁺ transformed epithelial cells in KC (FIG. 8, Panel G) and KPC tumors (FIG. 8, Panel H) expressed Dectin-1 ligands in vivo as did KPC-derived tumor cells grown in culture (FIG. 8, Panel I). Collectively, the data suggest high expression of the Dectin-1 receptor and Dectin-1 ligands in the epithelial and inflammatory compartments of PDA along with upregulation of associated signaling intermediates.

### Dectin-1 Ligation Accelerates Pancreatic Oncogenesis

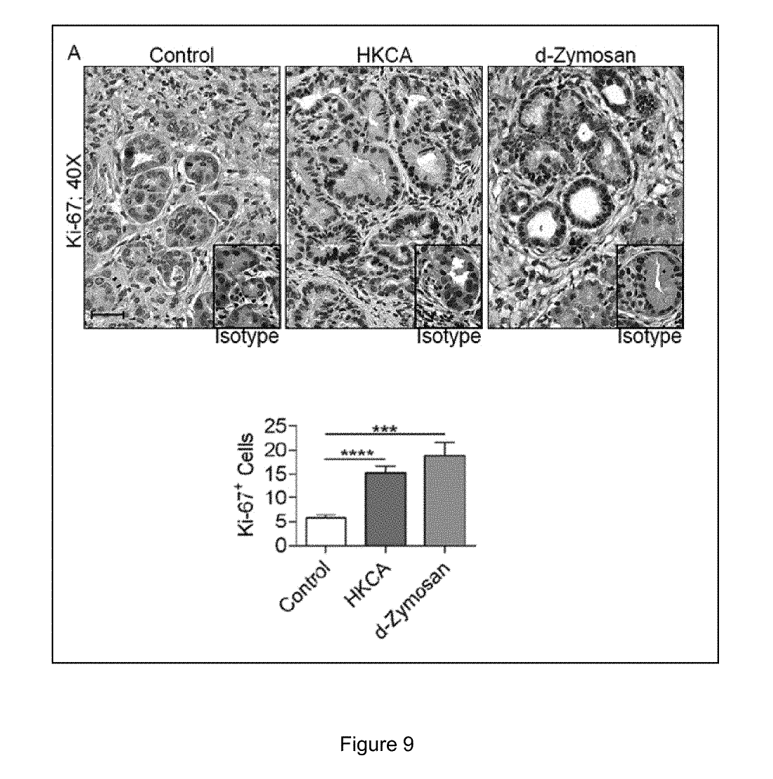

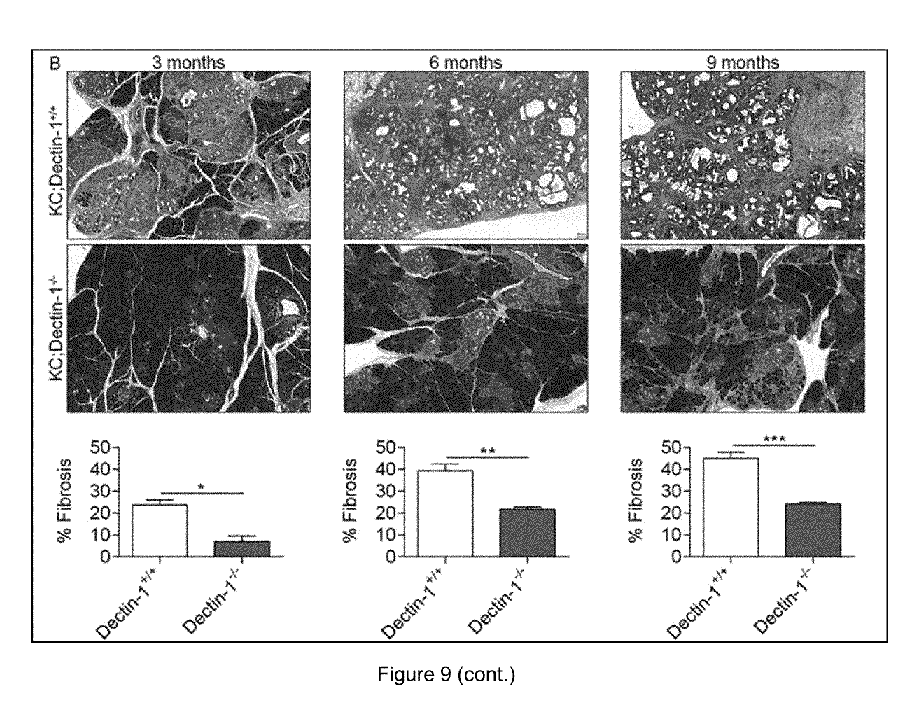

Since Dectin-1 and its cognate ligands are highly expressed in PDA, it was postulated that Dectin-1 signaling may promote immune-suppressive inflammation leading to accelerated tumorigenesis. To test this, six week-old KC mice were serially treated with the Dectin-1 specific agonists depleted Zymosan (d-Zymosan) or Heat-killed Candida albicans (HKCA) and tumor progression was assessed eight weeks later compared to vehicle-treated animals. Ligation of Dectin-1 vigorously accelerated tumorigenesis (FIG. 1, Panels F to I). Whereas pancreata in vehicle-treated KC mice harbored large areas of residually normal acinar architecture, mice treated with Dectin-1 agonists exhibited near-complete effacement of their pancreatic acini with more advanced PanIN lesions and numerous foci of invasive carcinoma embedded in dense fibro-inflammatory stroma (FIG. 1, Panels F to I). Ki67 proliferative rates were examined in six week-old KC mice treated with the Dectin-1 ligands d-Zymosan, HKCA, or vehicle for 8 weeks before sacrifice. The rate of epithelial cell proliferation was calculated based on Ki67 staining. Kras-transformed ductal epithelial cells in Dectin-1 agonist-treated KC mice also exhibited elevated Ki67 proliferative rates (FIG. 9, Panel A). Similarly, in vivo administration of Dectin-1 agonists d-Zymosan or HKCA accelerated tumor growth in orthotopically implanted KPC-derived tumors compared to administration of vehicle control, as shown by pancreatic tumor weight three weeks after administration (FIG. 1, Panel J). These data suggest that Dectin-1 signaling promotes PDA progression.

### Dectin-1 Deletion is Protective Against PDA

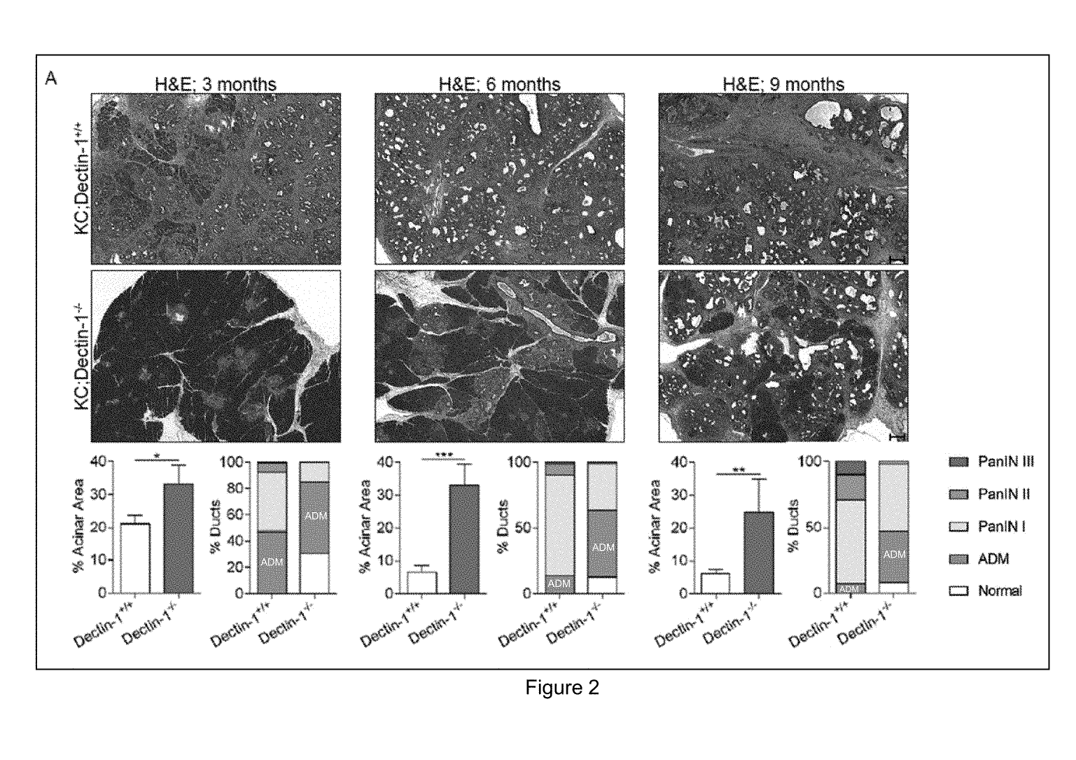

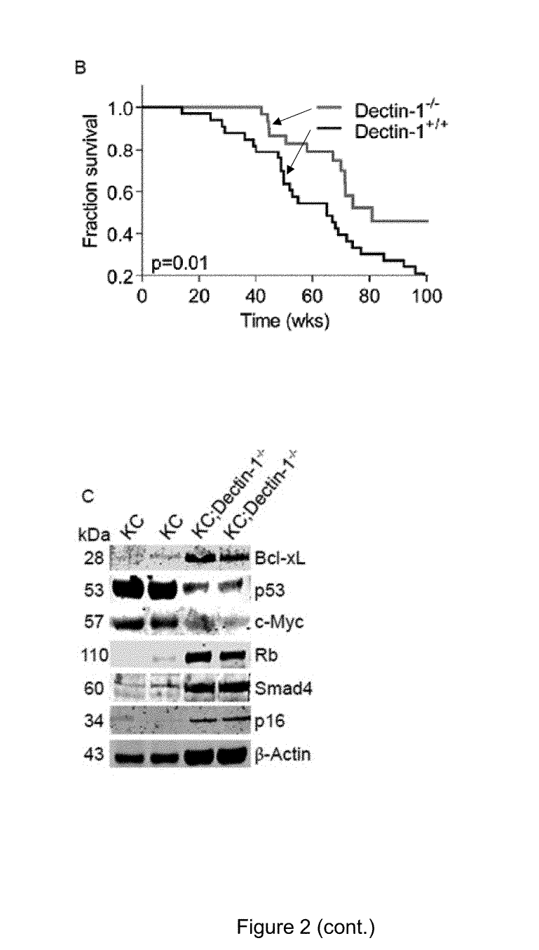

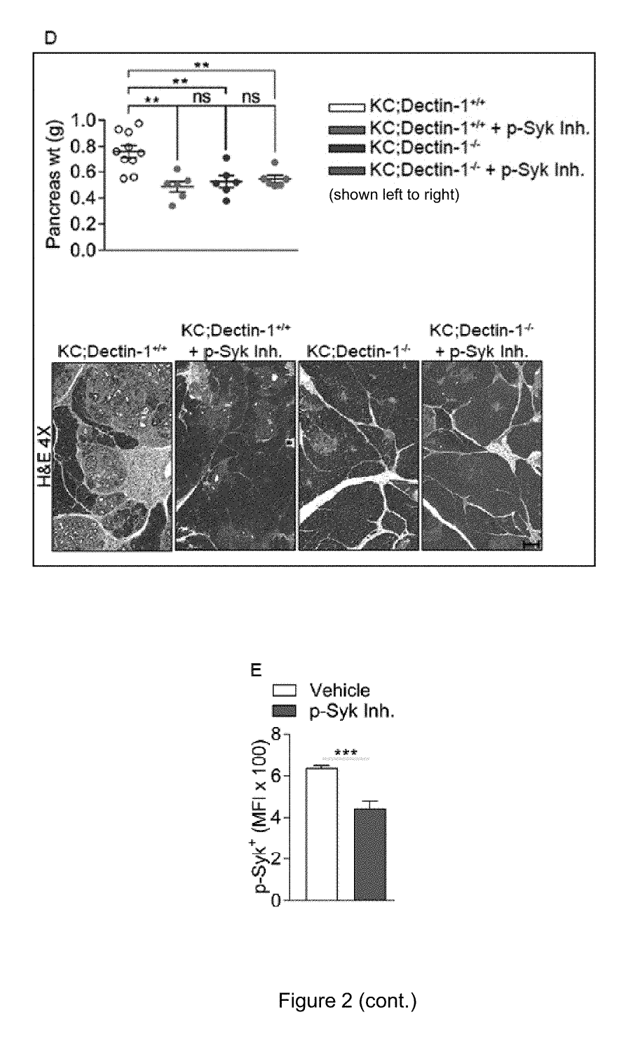

To determine whether Dectin-1 signaling is required for the normal progression of pancreatic oncogenesis, the tumor-phenotype in KC; Dectin-1^{-/-} mice was examined over time. Dectin-1 deletion delayed malignant progression and stromal expansion. Compared with KC controls, age-matched KC; Dectin-1^{-/-} pancreata exhibited delayed development of pancreatic dysplasia and fibrosis (FIG. 2, Panel A and FIG. 9, Panel B) and extended survival (FIG. 2, Panel B). To determine whether Dectin-1 deletion influences molecular oncogenesis, pancreata from KC and KC; Dectin-1^{-/-} mice were probed for select cell cycle regulatory, oncogenic, and tumor suppressor genes. KC; Dectin-1^{-/-} pancreata exhibited higher expression of Bcl-xL, Rb, Smad4, and p16 but reduced p53 and c-Myc expression suggesting a distinct oncogenic phenotype (FIG. 2, Panel C). Collectively, these data imply that Dectin-1 contributes to the normal progression of pancreatic neoplasia in the context of a driving Kras mutation.

### Syk Inhibition is Protective Against PDA

Since Dectin-1 signals via Syk phosphorylation, and it was shown that Syk activation is reduced in KC; Dectin-1^{-/-} pancreata, it was postulated that Syk blockade would be protective against pancreatic oncogenesis. KC mice were treated from 6-14 weeks of life with Piceatannol, a p-Syk inhibitor, and tested for tumor progression compared with vehicle-treated controls. It was confirmed that Piceatannol prevented Syk activation in vivo in PDA. Six week-old KC; Dectin-1^{+/+} and KC; Dectin-1^{-/-} mice were serially treated with the p-Syk inhibitor Piceatannol or vehicle for 8 weeks before sacrifice (n=5-10/group) (FIG. 2, Panel D). Syk inhibition was shown to reduce pancreatic tumor weight in the KC; Dectin-1^{+/+} group, but had no effect on the KC; Dectin-1^{-/-} mice. Similarly, WT mice bearing orthotopic PDA were serially treated with the p-Syk inhibitor Piceatannol or vehicle for 3 weeks. Tumor-infiltrating APC were harvested and tested for p-Syk expression by flow cytometry (FIG. 2, Panel E). Syk inhibition reduced pancreatic tumor weights and mitigated dysplastic changes but was not protective in KC; Dectin-1^{-/-} mice (FIG. 2, Panels D and E), suggesting that blockade of signaling pathways downstream of Dectin-1 may be an attractive therapeutic strategy in pancreatic oncogenesis.

Dectin-1 does not have Direct Pro-Tumorigenic Effects on Transformed Pancreatic Ductal Epithelial Cells

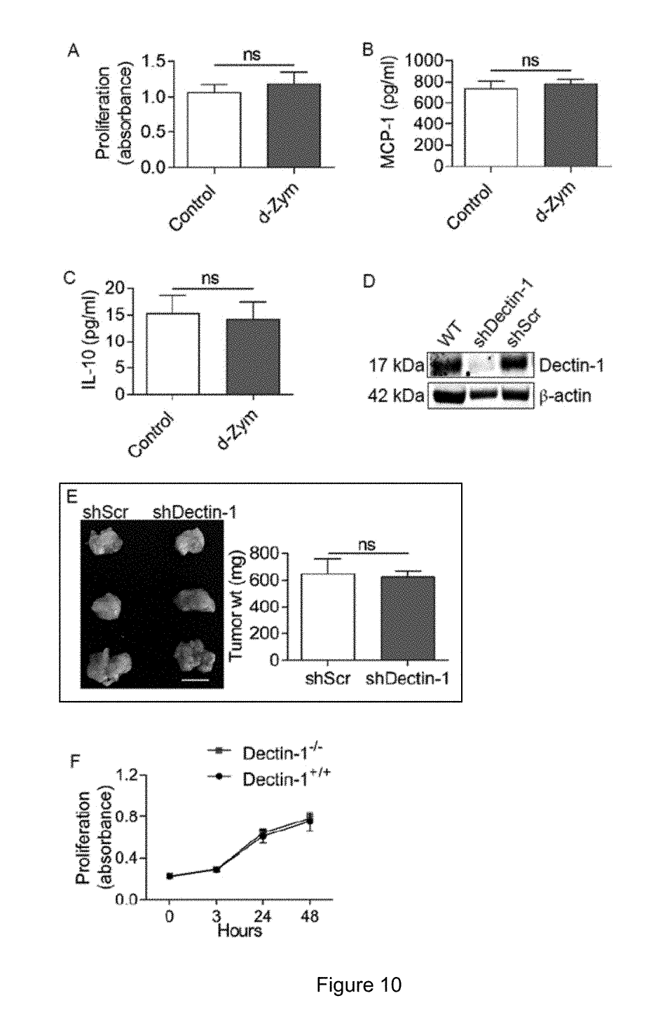

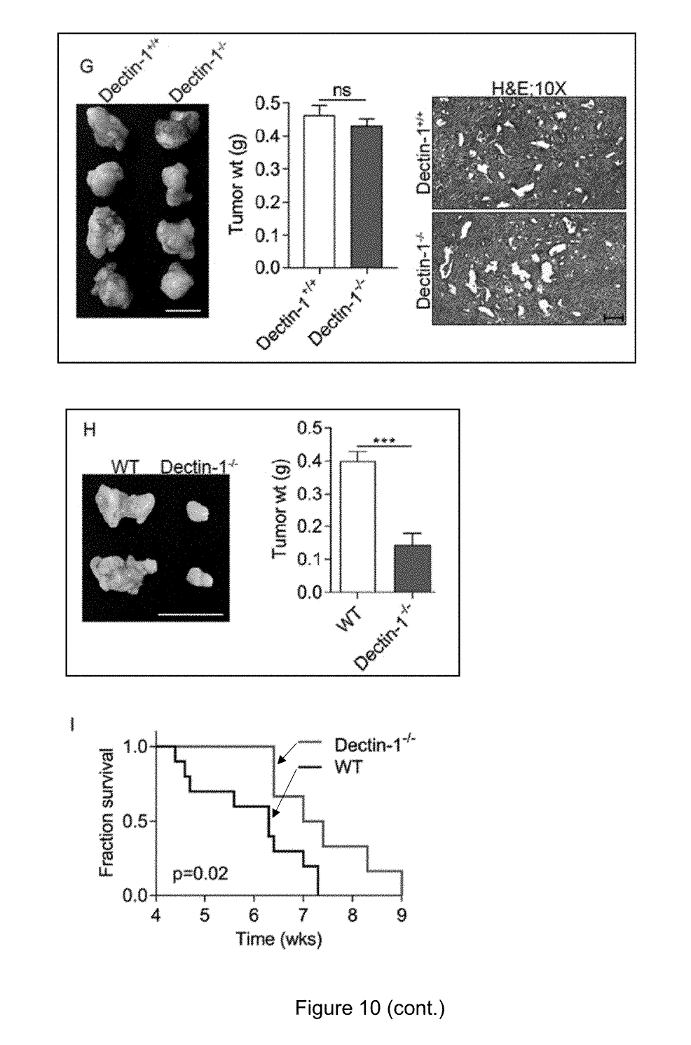

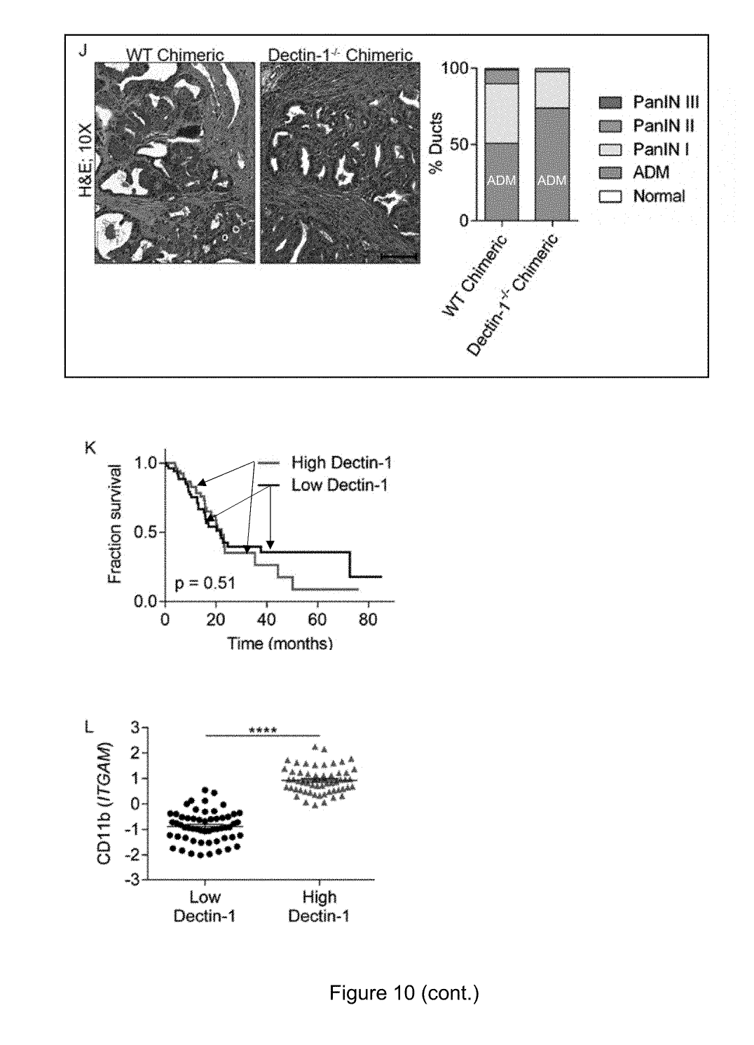

To determine whether Dectin-1 ligation has direct mitogenic or activating effects on transformed pancreatic epithelial cells, KPC-derived tumor cells were treated with vehicle or the Dectin-1 agonist d-Zymosan in vitro. The cellculcture supernatant was tested 48 hours later, and showed that Dectin-1 ligation failed to induce proliferation or cytokine production in PDA tumor cells (FIG. 10, Panels A to C). Experiments were performed in quadruplicate and repeated three times. HKCA similarly failed to induce proliferation or cytokine production in KPC cells. To further test whether Dectin-1 has direct oncogenic or pro-inflammatory effects in PDA cells, Dectin-1 expression was silenced in KPC-derived tumor cells using shRNA and confirmed the efficiency of the Dectin-1 knockdown with Western blotting (FIG. 10, Panel D). However, Dectin-1 knockdown did not alter the growth rate of tumor cells in vivo (FIG. 10, Panel E), as wild type mice orthotopically implanted with KPC-derived tumor cells treated with shRNA against Dectin-1 or with control scrambled shRNA did not show an appreciable difference. This finding suggests that Dectin-1 signaling in the transformed epithelial compartment is not critical in modulating PDA. Similarly, transformed pancreatic ductal epithelial cells (PDEC) harvested from 3 month-old KC and KC; Dectin-1^{-/-} pancreata proliferated at equal rates in vitro as measured by the XTT assay (FIG. 10, Panel F). To investigate proliferation rates in vivo, PDEC harvested from 3 month-old KC and KC; Dectin-1^{-/-} pancreata were cultured in vitro and then orthotopically implanted in pancreata of wild type mice. Mice were sacrificed at 21 days. Weights of Dectin-1^{+/+} and Dectin-1^{-/-} PDEC-derived tumors were compared. There was no appreciable difference between the two groups (FIG. 10, Panel G).

### Dectin-1 Deletion in the Extra-Epithelial Compartment Alone is Protective Against Oncogenesis

Since Dectin-1 ligation or knockdown does not influence the proliferative capacity of transformed pancreatic epithelial cells, it was postulated that Dectin-1 deletion in the extra-epithelial compartment alone would be protective against PDA. To investigate this, WT and Dectin-1^{-/-} mice were challenged with orthotopic injections of KPC-derived tumor cells with intact Dectin-1 expression. Pancreatic tumors harvested at 3 weeks were markedly smaller in Dectin-1^{-/-} hosts, suggesting that Dectin-1 deletion in the extra-tumoral compartment alone is protective against PDA (FIG. 10, Panel H). Dectin-1^{-/-} mice also exhibited extended survival after orthotopic PDA tumor implantation compared with WT mice (FIG. 10, Panel I). Similarly, KC mice made chimeric using Dectin-1^{-/-} bone marrow were protected against oncogenesis compared with KC mice made chimeric using WT bone marrow, confirming that deletion of Dectin-1 in leukocytes alone is protective (FIG. 10, Panel J). Dectin-1 expression was not associated with adverse survival in human PDA as shown by high vs. low tertile levels of Dectin-1 expression using the UCSC RNAseq database (FIG. 10, Panel K); however, Dectin-1 was a surrogate for total myeloid cell infiltration (FIG. 10, Panel L).

### Dectin-1 Deletion Induces Immunogenic Reprogramming of Tumor-Infiltrating Macrophages

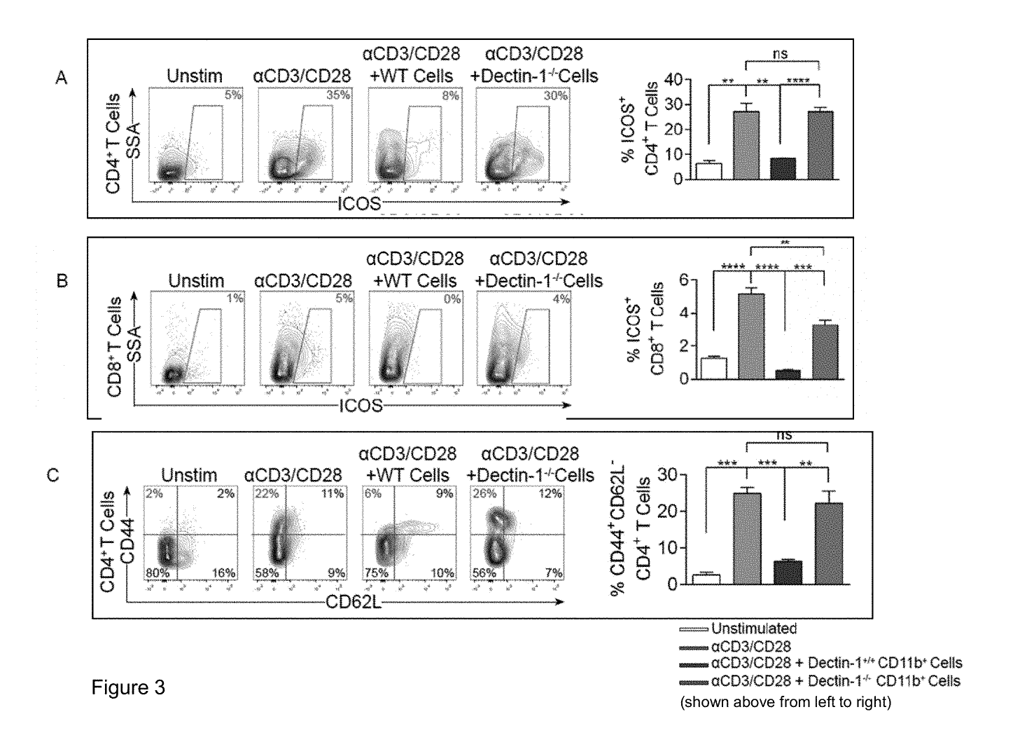

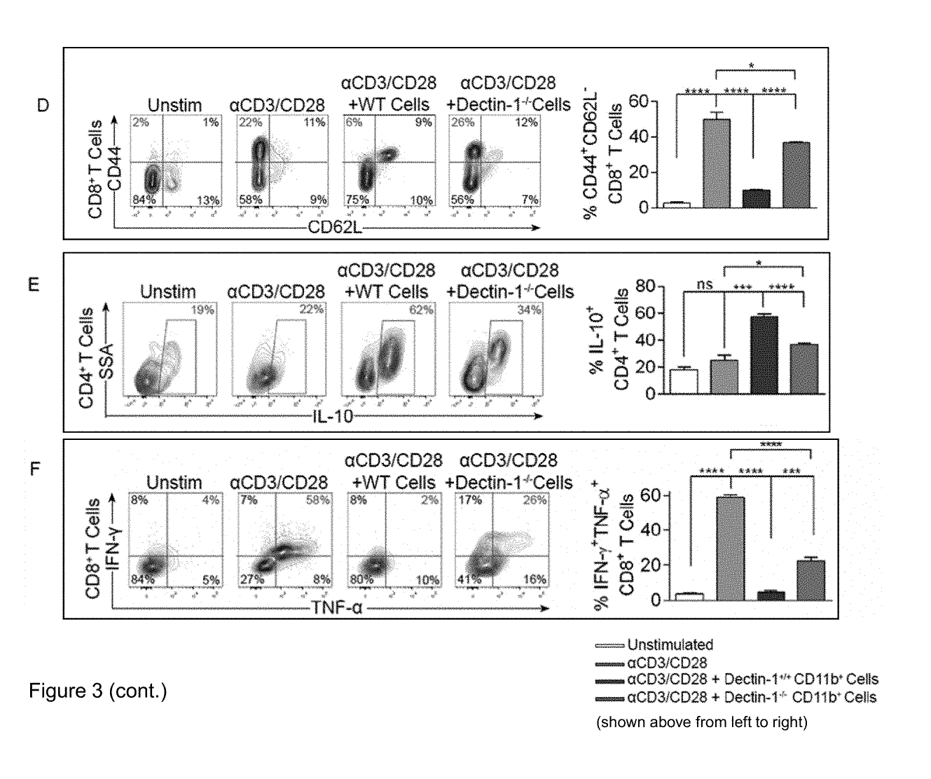

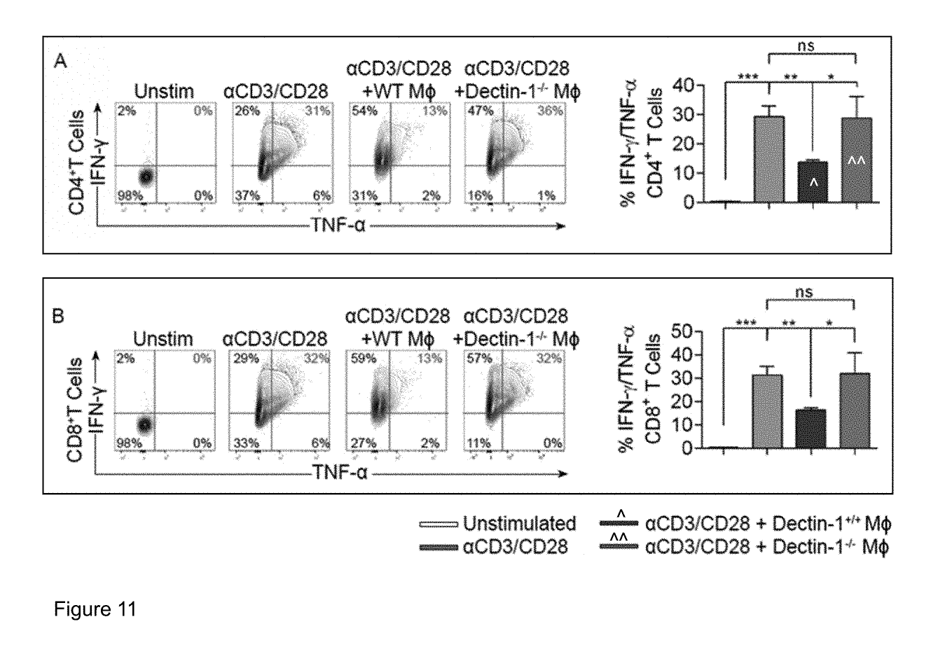

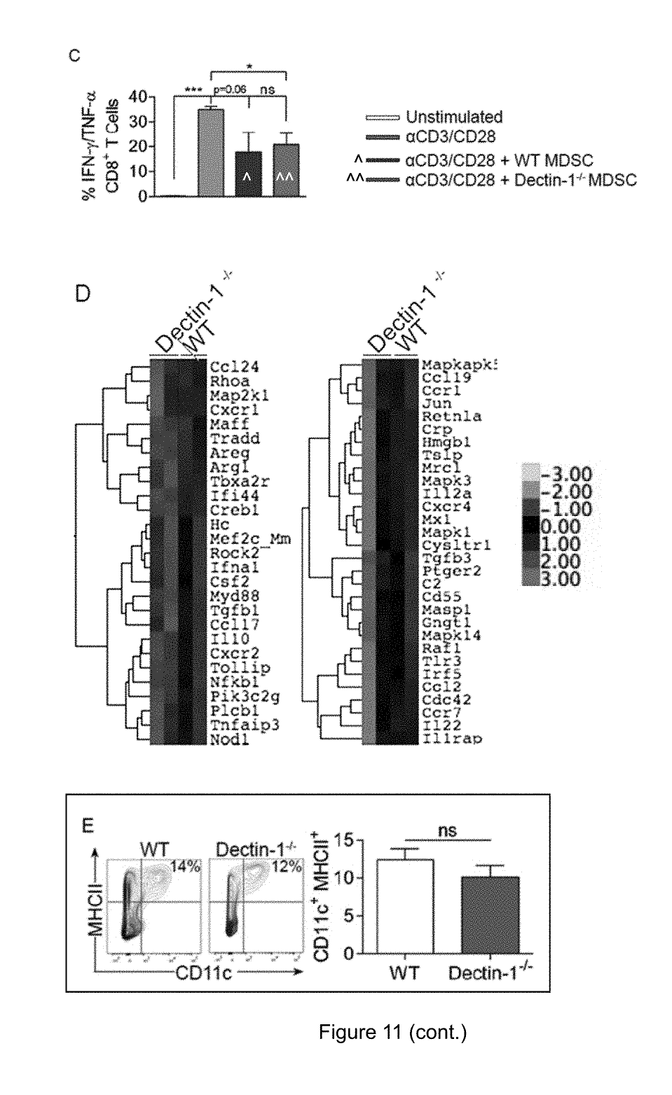

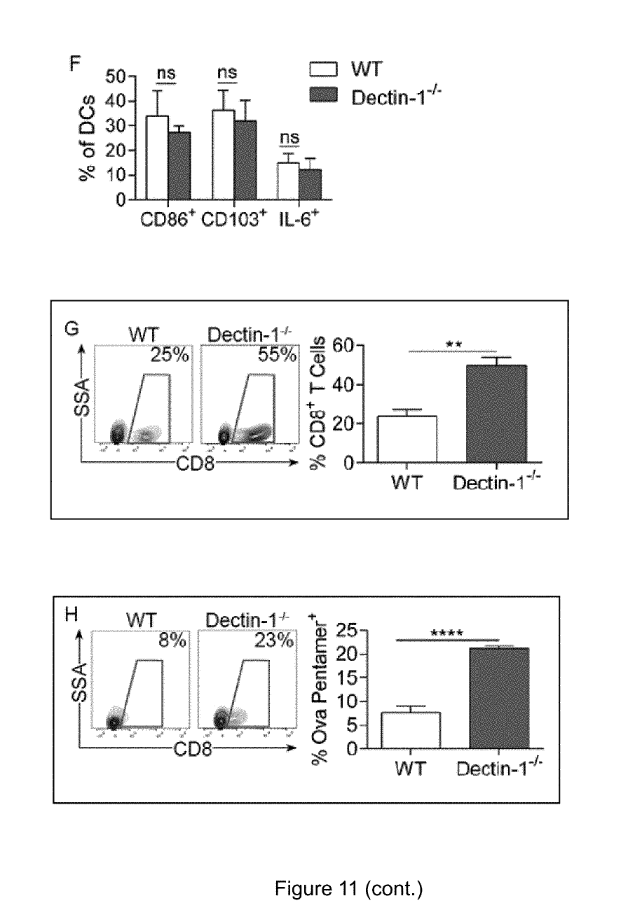

It was speculated that blockade of Dectin-1 signaling in the stroma leads to protection against PDA by bolstering anti-tumor immunity. Specifically, it was postulated that Dectin-1 deletion leads to immunogenic reprogramming of macrophages resulting in the reversal of the immune-suppressive phenotype of PDA-infiltrating T cells. To test this in vitro, naïve CD4⁺ and CD8⁺ T cells were stimulated using CD3/CD28 co-ligation. The expression of ICOS, CD44, IFN-γ, and TNF-α, which are upregulated on activated T cells, and the expression of CD62L and IL-10, which are downregulated upon T cell activation, were measured. Dectin-1^{+/+} or Dectin-1^{-/-} CD11b⁺ cells harvested from KPC-derived tumors were added to select wells. Whereas tumor-infiltrating WT myeloid cells abrogated ICOS upregulation in αCD3/CD28-activated CD4⁺ and CD8⁺ T cells, PDA-infiltrating Dectin-1^{-/-} myeloid cells exhibited minimal inhibitory effects (FIG. 3, Panels A and B). Similarly, whereas WT CD11b⁺ cells prevented CD4⁺ and CD8⁺ T cell adoption of a CD44+CD62L⁻ effector memory phenotype in response to CD3/CD28 ligation, Dectin-1^{-/-} cells were non-inhibitory (FIG. 3, Panels C and D). Moreover, in contrast to tumor-infiltrating WT CD11b⁺ cells which promoted 1-10 production from CD4⁺ T cells (FIG. 3, Panel E) and negated IFN-γ and TNF-α expression in CD8⁺ T cells (FIG. 3, Panel F), Dectin-1^{-/-} cells only minimally induced IL-10 production in CD4⁺ T cells and were permissive of CD8⁺ cytotoxic T cell activation (FIG. 3, Panels E and F). Similar differential effects on CD4⁺ and CD8⁺ T cell inhibition, determined at 72h by co-expression of IFN-γ and TNF-α, were observed when using purified Gr1⁻CD11c⁻CD11b⁺F4/80⁺ PDA-associated macrophages (TAMs) harvested from WT versus Dectin-1^{-/-} hosts (FIG. 11, Panels A and B). Conversely, the T cell inhibitory capacity of Gr1⁺CD11b⁺ neutrophils and inflammatory monocytes was not diminished in the context of Dectin-1 deletion. CD8⁺ splenic T cells were either unstimulated, or stimulated with αCD3/αCD28 alone or in co-culture with PDA-infiltrating Gr1⁺CD11b⁺ neutrophils and inflammatory monocytes harvested from WT or Dectin-1^{-/-} hosts. T cell activation was determined at 72h as described above (FIG. 11, Panel C). These data suggest that only Dectin-1 signaling in TAMs influences T cell function.

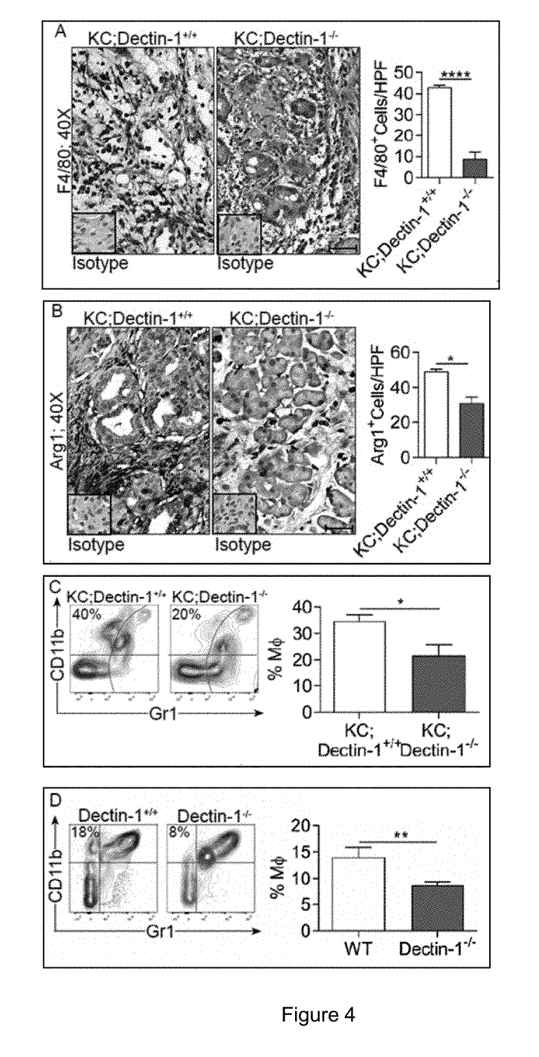

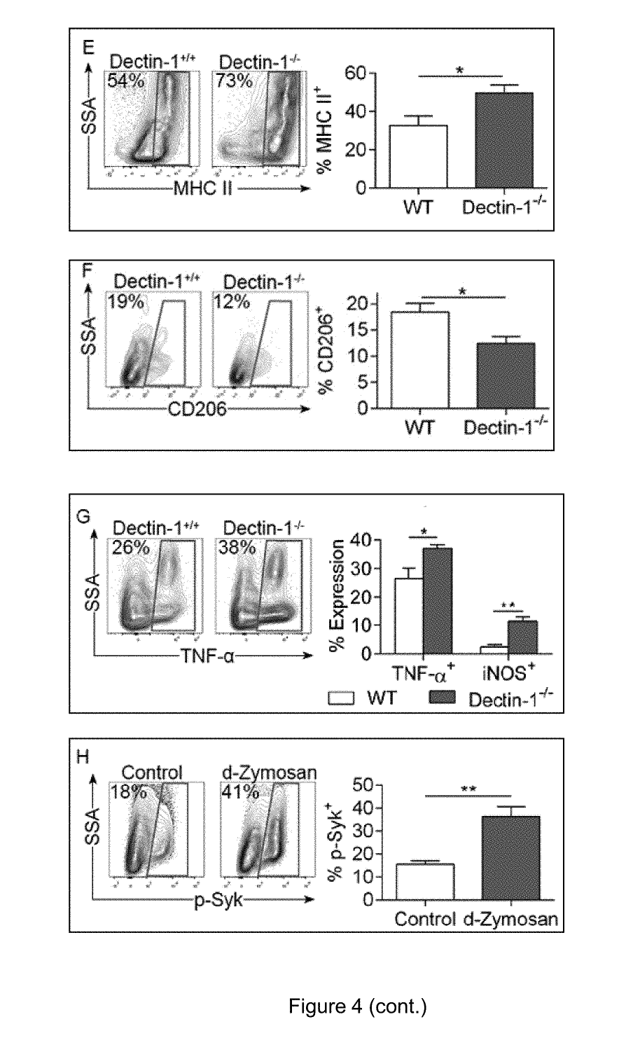

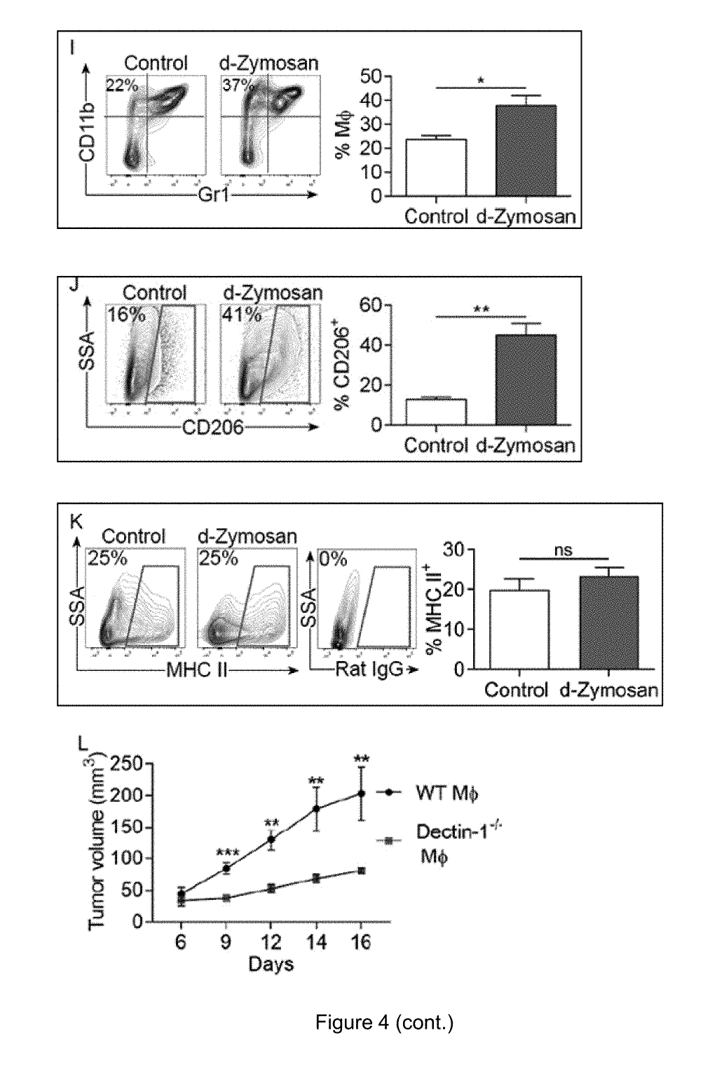

To investigate whether Dectin-1 signaling promotes macrophage-mediated immune-suppression in situ in PDA, macrophage recruitment and phenotype were assessed in both the slowly progressive (KC) and invasive (KPC) models of PDA in the contexts of either Dectin-1 deletion or activation. Six month-old KC; Dectin-1^{+/+} and KC; Dectin-1^{-/-} mice were tested for F4/80⁺ (FIG. 4, Panel A) and Arg1⁺ (FIG. 4, Panel B) macrophage infiltration. KC; Dectin-1^{-/-} mice exhibited significantly reduced pancreatic infiltration with F4/80⁺ and Arg1⁺ TAMs on IHC analysis. Flow cytometry confirmed an approximately 50% reduction in the fraction of TAMs in KC; Dectin-1^{-/-} pancreata (FIG. 4, Panel C). A similar decrease in the fraction of TAMs in orthotopic KPC tumors in Dectin-1^{-/-} hosts compared with WT (FIG. 4, Panel D). Accordingly, analysis of orthotopic KPC tumors using a PCR array suggested reduced expression of diverse inflammatory mediators in tumors in Dectin-1^{-/-} hosts compared with WT (FIG. 11, Panel D). Moreover, cellular phenotyping experiments indicated that Dectin-1 deletion induced immunogenic reprogramming of TAMs toward M1-like differentiation. Specifically, TAMs infiltrating Dectin-1^{-/-} pancreata expressed elevated MHC II, reduced CD206, and higher TNF-α and iNOS compared with Dectin-1^{+/+} hosts suggesting M1-like programming (FIG. 4, Panels E to G). By contrast, the prevalence and immune-phenotype of CD11c⁺MHCII⁺ DC was similar in both WT and Dectin-1^{-/-} PDA tumors (FIG. 11, Panels E and F).

Wild type mice were implanted with orthotopic KPC tumors and serially treated with the Dectin-1 specific ligand d-Zymosan or vehicle. It was confirmed that exogenous Dectin-1 ligand administration using d-Zymosan activates Syk signaling in the PDA TME (FIG. 4, Panel H). Accordingly, in vivo Dectin-1 ligation increased the fraction of TAMs in orthotopic KPC tumors (FIG. 4, Panel I), and upregulated CD206 expression (FIG. 4, Panel J); however, MHC II expression was not significantly altered (FIG. 4, Panel K). Similar effects on macrophage programming were seen after in vivo treatment with HKCA. Further, Dectin-1^{-/-} mice were subcutaneously implanted with KPC-derived PDA tumor cells admixed with WT or Dectin-1^{-/-} macrophages, and demonstrated that adoptive transfer of WT macrophages coincident with PDA tumor challenge in Dectin-1^{-/-} hosts results in an accelerated tumor growth rate compared with adoptive transfer of Dectin-1^{-/-} macrophages (FIG. 4, Panel L).

### Dectin-1 Signaling Suppresses T Cell Immunogenicity in PDA

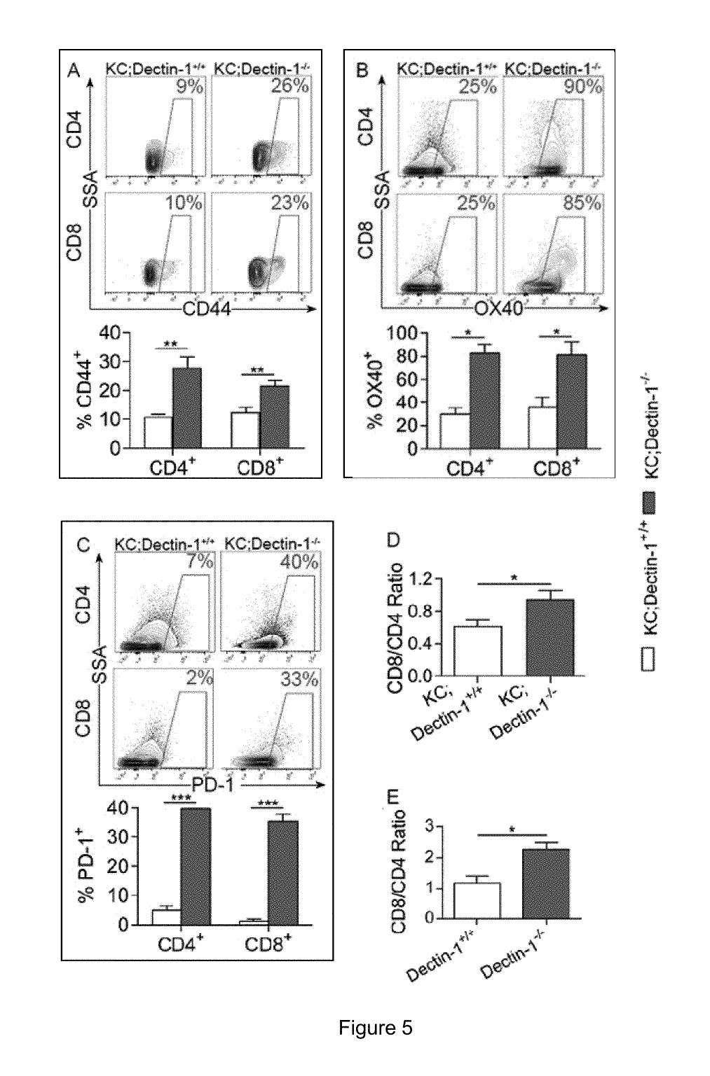

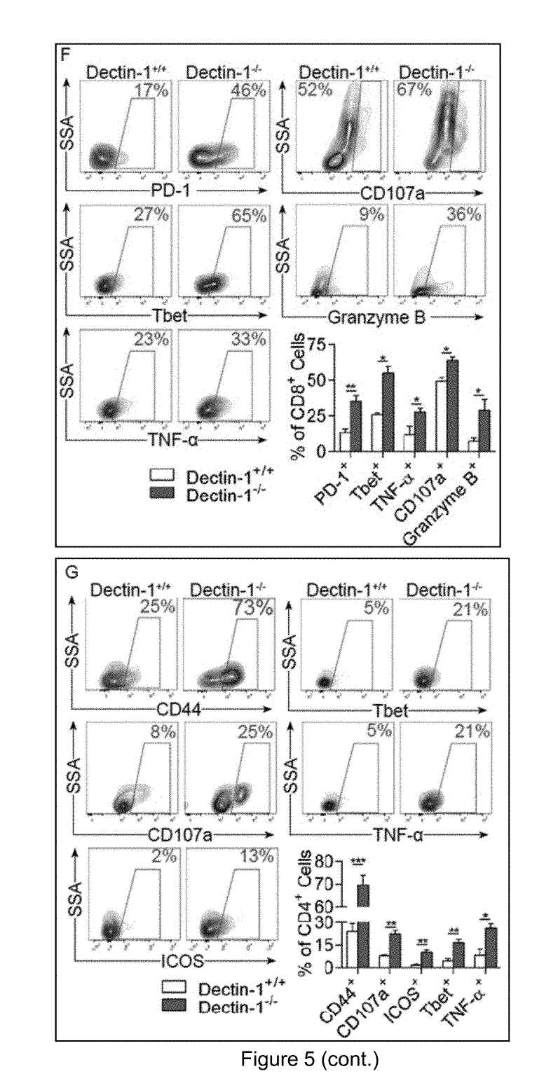

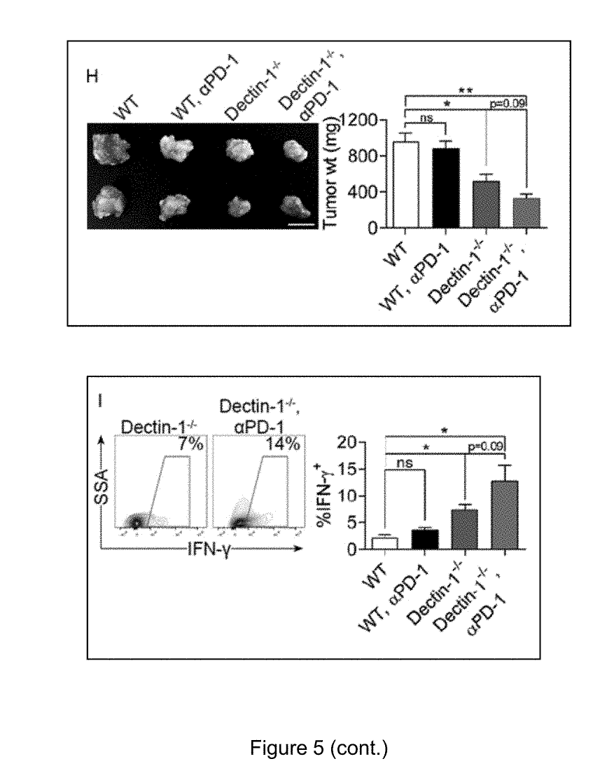

Based on the macrophage phenotyping and adoptive transfer experiments and the in vitro co-culture data, it was postulated that the reprogramming of TAMs resulting from Dectin-1 deletion in PDA leads to enhanced immunogenicity in tumor-entrained T cells. To test this, the T cell phenotype was interrogated in inferior pancreas-draining lymph nodes in KC vs KC; Dectin-1^{-/-} mice, as well as in orthotopic KPC tumors in WT vs Dectin-1^{-/-} hosts. Consistent with the hypothesis, Dectin-1 deletion in KC mice led to immunogenic reprogramming of tumor-draining CD4⁺ and CD8⁺ T cells, which exhibited upregulated expression of CD44, OX40, and PD-1 indicative of cellular activation (FIG. 5, Panels A to C). Dectin-1 deletion also increased the CD8:CD4 ratio in tumor draining lymph nodes (FIG. 5, Panel D). Further, wild type and Dectin-1^{-/-} mice were challenged with orthotopic KPC tumors, and Dectin-1 deletion was shown to increase the CD8:CD4 ratio in tumor-infiltrating T cells in orthotopic KPC tumors (FIG. 5, Panel E). Additionally, CD8⁺ T cells in KPC tumors in Dectin-1^{-/-} hosts exhibited an activated phenotype with high expression of PD-1, T-bet, TNF-α, CD107a, and Granzyme B, suggesting enhanced cytotoxic potential compared with orthotopic KPC tumors in WT hosts (FIG. 5, Panel F). Accordingly, orthotopic administration of KPC cells engineered to express OVA resulted in a markedly higher fraction of tumor-infiltrating OVA Pentamer⁺ cytotoxic T cells in Dectin-1^{-/-} hosts compared with WT (FIG. 11, Panels G and H). Similarly, PDA-infiltrating CD4⁺ T cells in Dectin-1^{-/-} hosts expressed higher CD44, CD107a, and ICOS and exhibited enhanced Th1 polarization as evidenced by upregulated expression of T-bet and TNF-α (FIG. 5, Panel G). Collectively, these data indicate enhanced T cell immunogenicity. Notably, consistent with higher T cell activation and PD-1 expression in PDA tumors in Dectin-1^{-/-} mice, combined Dectin-1 deletion+PD-1 blockade (αPD-1) trended to offer synergistic protection and further enhanced intra-tumoral Th1 polarization whereas PD-1 blockade had no efficacy in absence of Dectin-1 deletion (FIG. 5, Panels H and I).

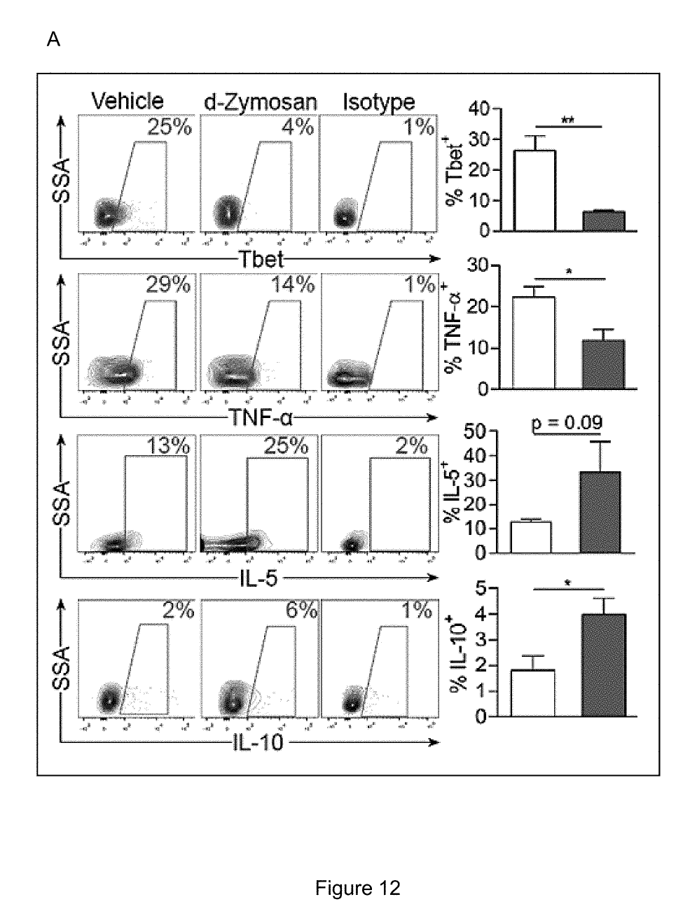

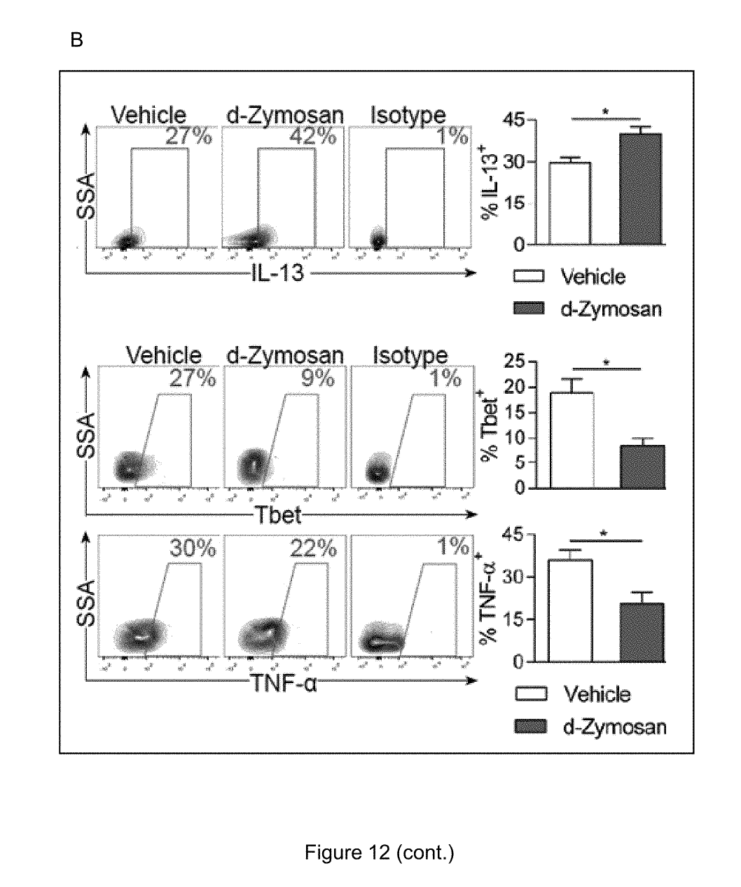

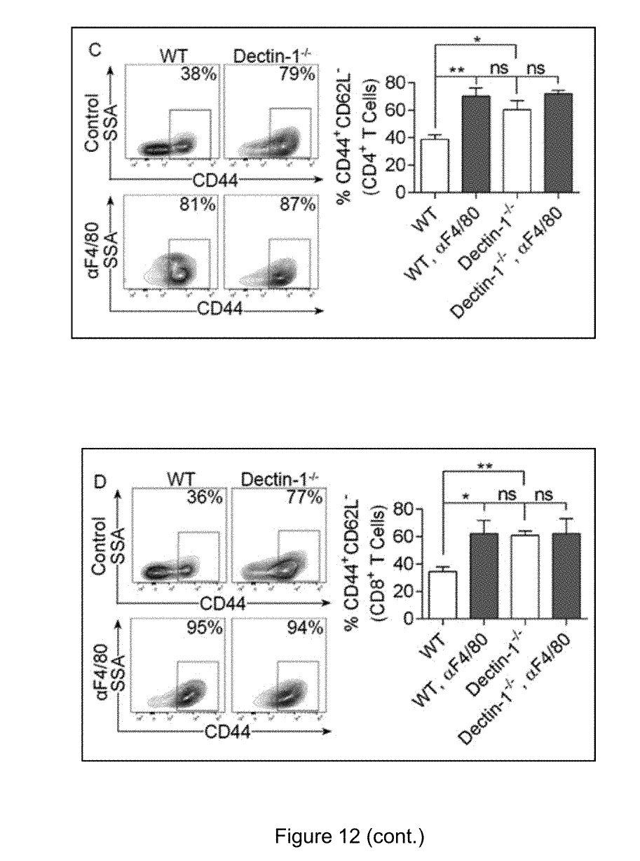

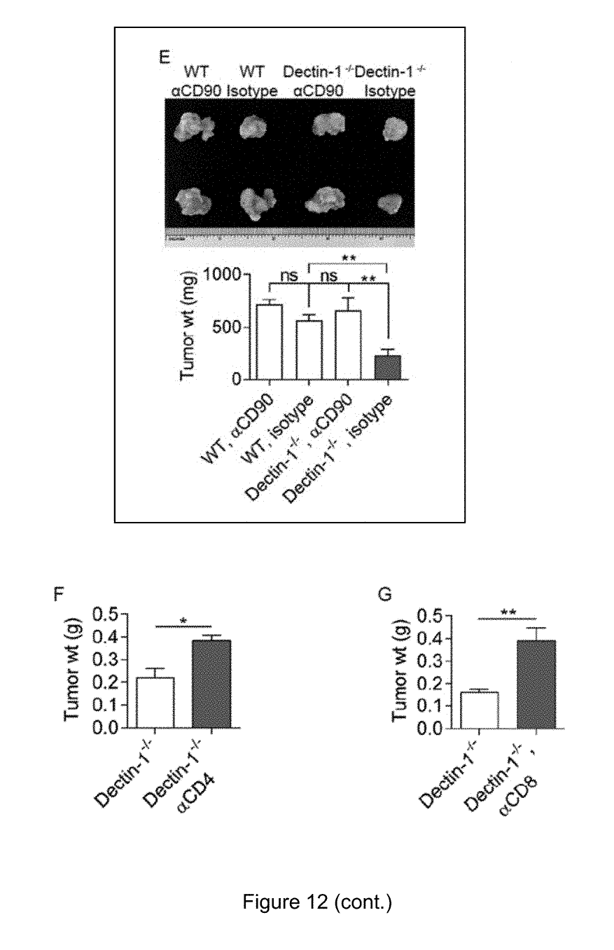

It was postulated that exogenous Dectin-1 ligation would induce an immune-suppressive Th2 phenotype and reduce CD8⁺ T cell activation. Accordingly, CD4⁺ T cells harvested from d-Zymosan treated KPC-tumor bearing mice exhibited reduced T-bet and TNF-α expression but higher IL-5, IL-10, and IL-13 expression (FIG. 12, Panel A). Similarly, CD8⁺ T cells exhibited diminished T-bet and TNF-α expression after Dectin-1 ligation (FIG. 12, Panel B). Further, cohorts of wild type and Dectin-1^{-/-} animals were challenged with orthotopic PDA and serially treated with a neutralizing αF4/80 mAb or isotype control, which demonstrated that in vivo macrophage depletion activated PDA-infiltrating CD4⁺ and CD8⁺ T cells exclusively in WT hosts but not in Dectin-1^{-/-} hosts, suggesting that Dectin-1-expressing macrophages drive T cell suppression in PDA (FIG. 12, Panels C and D).

To definitively test whether tumor-protection in the absence of Dectin-1 signaling is contingent on immunogenic T cell reprogramming, T cells were depleted coincident with orthotopic KPC tumor administration in cohorts of WT and Dectin-1^{-/-} animals. The cohorts of wild type and Dectin-1^{-/-} animals were serially treated with a neutralizing αCD90 monoclonal antibody or isotype control. Pan-T cell depletion did not affect PDA growth in WT mice; however, tumor protection was abrogated in Dectin-1^{-/-} cohorts (FIG. 12, Panel E). Dectin-1^{-/-} mice were challenged with orthotopic PDA and serially treated with neutralizing αCD4 (FIG. 12, Panel F) or αCD8 (FIG. 12, Panel G) monoclonal antibodies or isotype. Similarly, CD4⁺ and CD8⁺ T cell depletion alone each reversed tumor-protection in Dectin-1^{-/-} mice (FIG. 12, Panels F and G) but not in WT. These data suggest that in PDA-bearing WT hosts, T cells are dispensable to outcome; conversely, in the absence of Dectin-1 signaling, T cells are reprogrammed into indispensable mediators of tumor-protection.

### Galectin-9 Ligates Dectin-1 in PDA

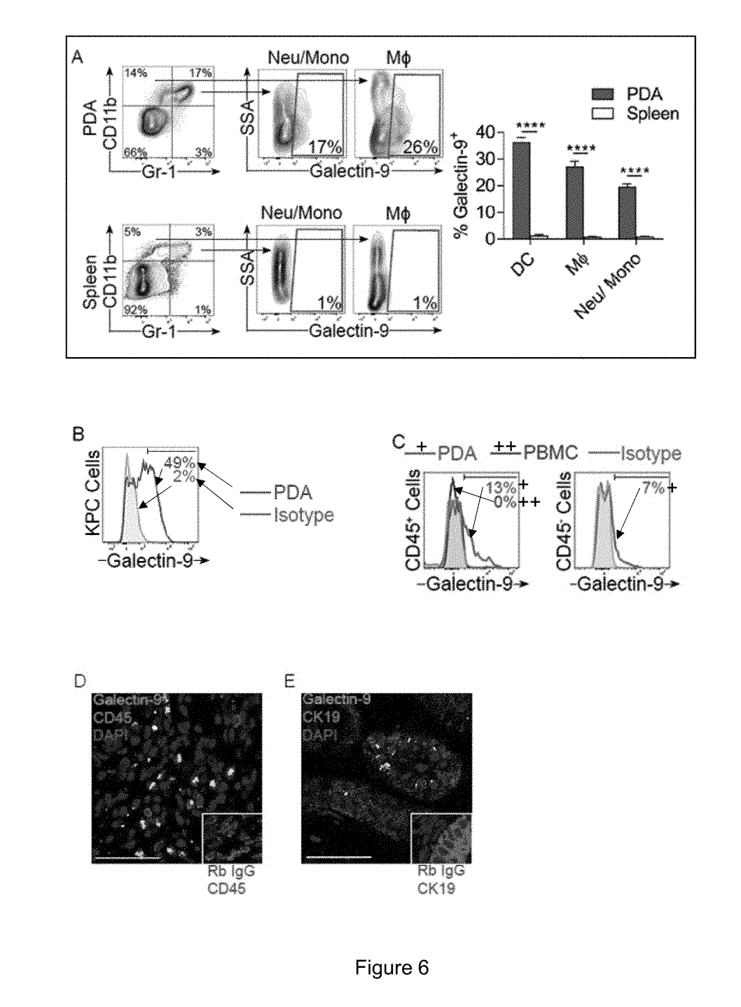

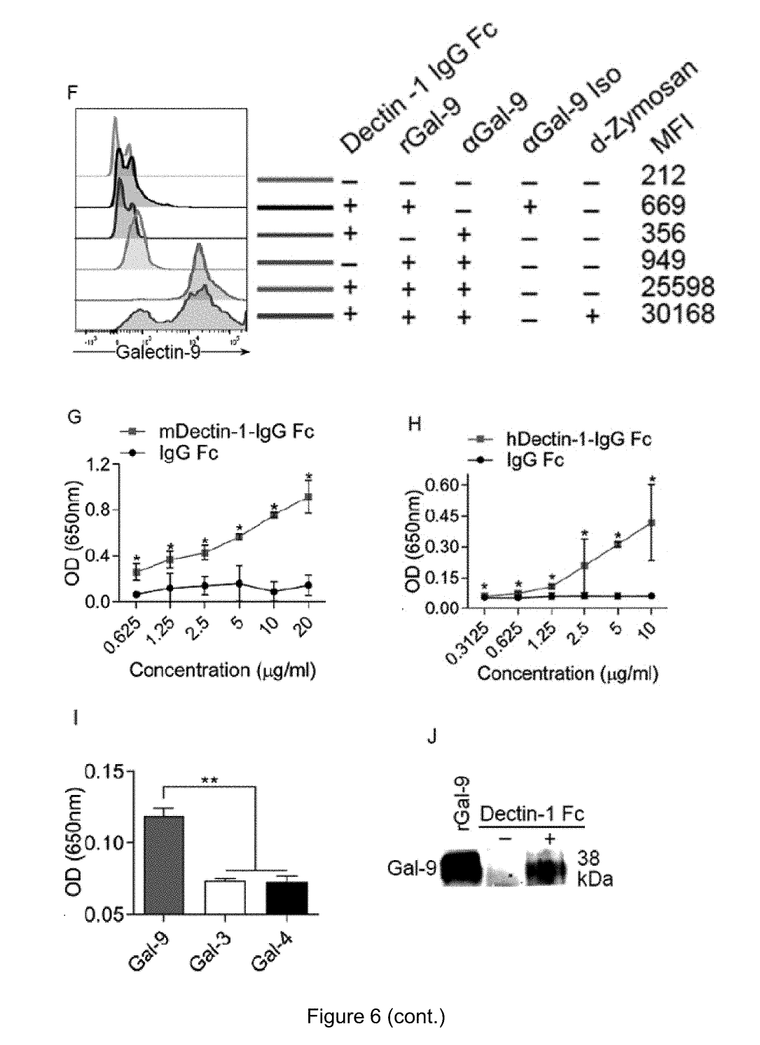

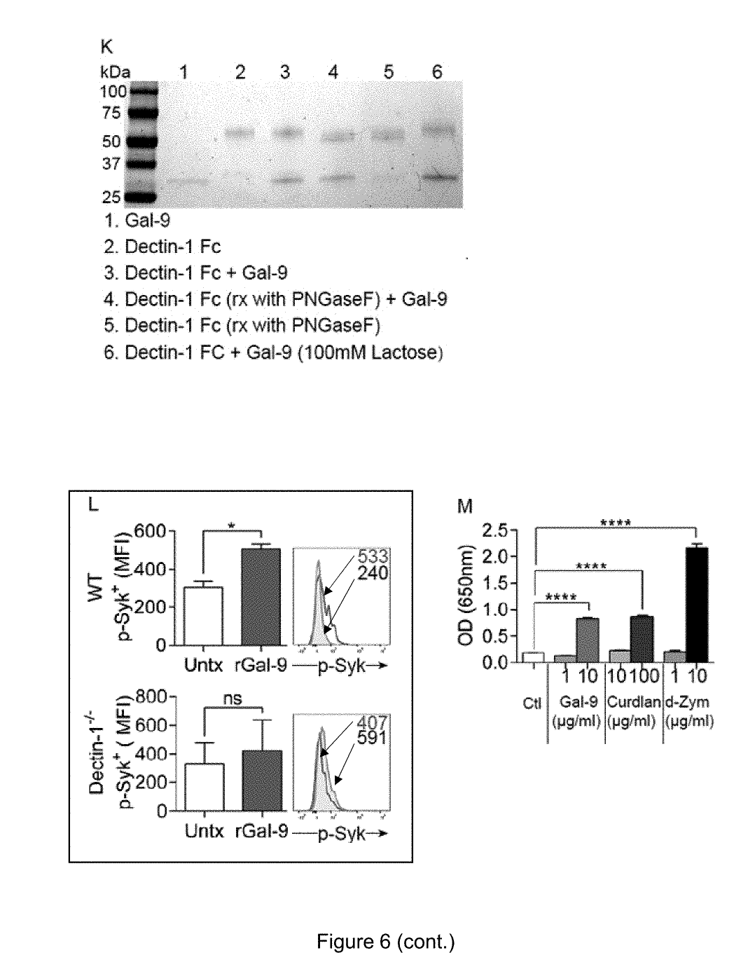

Non-pathogen derived Dectin-1 ligands have not been well-characterized. Therefore, affinity purification-mass spectrometry was performed using the IgG Fc-conjugated Dectin-1 fusion protein coupled to protein G beads to purify putative ligand(s) in KPC tumor extracts. The proteins co-purified with the Dectin-1 fusion protein were contrasted with proteins purified with protein G beads alone. Affinity purification coupled with mass spectrometry experiments were repeated twice and only proteins that uniquely co-purified with the IgG Fc-conjugated Dectin-1 fusion protein were considered possible candidate Dectin-1 ligands. A total of 19 proteins were identified. Among the co-purified proteins was Galectin-9 (Table 1). Since Galectin-9 is a member of the β-galactoside-binding family of lectins, it was hypothesized that Galectin-9 is a sterile ligand for Dectin-1. The presence of Galectin-9 was assayed for in the murine PDA TME and robust expression of Galectin-9 was found in diverse PDA-infiltrating myeloid cells and in cancer cells by flow cytometry (FIG. 6, Panels A and B). Modest expression of Galectin-9 was also found in both leukocytes and tumor cells in human PDA, whereas Galectin-9 was minimally expressed in leukocytes in PBMC (FIG. 6, Panel C). Expression of Galectin-9 was further demonstrated in PDA-infiltrating leukocytes (13%) and cancer cells (7%) by confocal microscopy (FIG. 6, Panels D and E). To investigate whether Dectin-1 ligates Galectin-9, protein G-magnetic beads were loaded with the Dectin-1 IgG Fc fusion protein or control IgG Fc. Bead-IgG Fc complexes were incubated with recombinant Galectin-9 and then labeled with a fluorescently-conjugated αGalectin-9 mAb and tested for fluorescence by flow cytometry. Controls included: unstained beads, bead-IgG Fc complexes+recombinant Galectin-9+fluorescently-conjugated isotype antibody, bead-IgG Fc complexes+fluorescently-conjugated anti-Galectin-9, beads without Dectin-1 IgG Fc incubated with recombinant Galectin-9+fluorescently-conjugated anti-Galectin-9. To test for competitive inhibition of Galectin-9 binding with a well-characterized Dectin-1 ligand, the bead-IgG Fc complexes were incubated with recombinant Galectin-9 together with d-Zymosan and then stained with fluorescently-conjugated anti-Galectin-9. This assay was repeated twice with similar results. It was found that the IgG Fc-conjugated Dectin-1 fusion protein avidly ligated Galectin-9 whereas controls failed to elicit a positive signal (FIG. 6, Panel F). Furthermore, the data suggest that Galectin-9 ligation of Dectin-1 may be competitively inhibited by the well-characterized Dectin-1 ligand d-Zymosan (FIG. 6, Panel F). Additionally, Galectin-9 coated ELISA plates were incubated with increasing doses of murine or human Dectin-1 IgG Fc or control IgG Fc. The Galectin-9-bound Dectin-1 IgG Fc was detected with anti-IgG-HRP. Galectin-9 was also found to bind murine (FIG. 6, Panel G) and human (FIG. 6, Panel H) Dectin-1 in a dose-dependent manner on ELISA. However, when Galectin-3, Galectin-4, and Galectin-9 coated ELISA plates were incubated with Dectin-1 IgG Fc or control IgG Fc (2.5 μg/ml) in parallel, and Galectin-bound Dectin-1 IgG Fc was detected with anti-IgG-HRP, murine Dectin-1 did not avidly bind Galectin-3 or Galectin-4 (FIG. 6, Panel I). The binding of Galectin-9 to Dectin-1 in situ was further confirmed by precipitating Dectin-1 ligands in pancreatic tissue extract from 6 month-old KC mice using the Dectin-1 IgG Fc or control IgG Fc and then probing for Galectin-9 by Western blotting. The results indicated Dectin-1-Galectin-9 complex formation (FIG. 6, Panel J). Given that Galectin-9 binds to polylactosamine epitopes on glycoproteins, whether Galectin-9 ligates Dectin-1 through a glycan/galectin-9 interaction was assessed. To test this, Dectin-1 was pretreated with PNGaseF, an enzyme that cleaves N-linked glycans, loaded onto Protein G beads and incubated with recombinant mouse Galectin-9 pre-incubated with 100 mM lactose or buffer. Whether this modulated the interaction was tested. No change in the binding between the proteins was observed, suggesting glycan-independent binding (FIG. 6, Panel K). In addition, Galectin-9 was pretreated with high concentrations of lactose, a known inhibitor of galectins, prior to incubation with Dectin-1. Again, no alteration of the Dectin-1/Galectin-9 interaction was observed confirming glycan-independent binding (FIG. 6, Panel K). To determine whether Galectin-9 is a functional Dectin-1 ligand, WT and Dectin-1^{-/-} macrophages were treated with recombinant Galectin-9 (10 ug/ml for 3 hours) and Syk phosphorylation was determined by flow cytometry compared with isotype control. Galectin-9 activated Syk in a Dectin-1-dependent manner (FIG. 6, Panel L). Similarly, Dectin-1 reporter HEK293 cells untreated or treated with low and high doses of Galectin-9 or well-characterized Dectin-1 ligands Curdlan and d-Zymosan, showed that Galectin-9 induced NF-κB signaling in a HEK293 Dectin-1 reporter cell line (Walachowski et al., PLoS One, 2016, 11:e0148464) in a dose dependent manner (FIG. 6, Panel M). An irrelevant ligand specific for the C-type lectin receptor Mincle did not activate the Dectin-1 reporter cells.

TABLE 1
Manually curated list of proteins exclusively identified in two different Dectin-1 pull
down experiments and not detected in the controls.

| | | IP1 | | | IP2 | | |
|---|---|---|---|---|---|---|---|
| | | Sequence | | | Sequence | | |
| | Gene | Coverage | Unique | # | Coverage | Unique | # |
| Protein Names | Names | % | Peptides | PSM | % | Peptides | PSM |
| Endoplasmin | Hsp90b1 | 29.68 | 23 | 30 | 11.85 | 7 | 8 |
| C-type lectin domain family | Clec7a | 27.05 | 5 | 25 | 42.21 | 9 | 98 |
| 7 member A | | | | | | | |
| Elongation factor 1-alpha 1 | Eef1a1 | 32.03 | 12 | 23 | 51.73 | 26 | 48 |
| Eef2 protein | Eef2 | 25.86 | 19 | 23 | 37.72 | 26 | 31 |
| Polyadenylate-binding | Pabpc1 | 21.23 | 11 | 13 | 55.03 | 36 | 92 |
| protein 1 | | | | | | | |
| Sodium/potassium- | Atp1a1 | 16.13 | 12 | 13 | 17.40 | 11 | 14 |
| transporting ATPase subunit | | | | | | | |
| alpha-1 | | | | | | | |
| Elongation factor 1-gamma | Eef1g | 21.74 | 10 | 11 | 26.54 | 9 | 11 |
| Galectin-9 | Lgals9 | 27.95 | 7 | 9 | 31.06 | 8 | 8 |
| Clusterin | Clu | 13.84 | 6 | 8 | 22.99 | 8 | 9 |
| Cytochrome b-c1 complex | Uqcrc2 | 16.56 | 6 | 8 | 24.94 | 8 | 8 |
| subunit 2, mitochondrial | | | | | | | |
| Probable ATP-dependent | Ddx17 | 10.77 | 7 | 7 | 38.92 | 25 | 36 |
| RNA helicase DDX17 | | | | | | | |
| Peroxiredoxin-1 | Prdx1 | 37.19 | 6 | 7 | 55.78 | 12 | 16 |
| Dolichyl- | Rpn1 | 14.83 | 7 | 7 | 30.15 | 12 | 14 |
| diphosphooligosaccharide- | | | | | | | |
| protein glycosyltransferase | | | | | | | |
| subunit 1 | | | | | | | |
| Isoform 2 of Microtubule- | Map4 | 8.99 | 6 | 6 | 20.20 | 14 | 23 |
| associated protein 4 | | | | | | | |
| Putative uncharacterized | Sqrdl | 19.33 | 6 | 6 | 34.44 | 16 | 19 |
| protein | | | | | | | |
| Prohibitin | Phb | 22.79 | 5 | 5 | 23.90 | 5 | 6 |
| 60S ribosomal protein L18 | Rp118 | 25.00 | 4 | 4 | 28.19 | 7 | 10 |
| Poly(A) binding protein, | Pabpc4 | 5.20 | 2 | 3 | 46.34 | 28 | 52 |
| cytoplasmic 4 | | | | | | | |

Table 1 shows a manually curated list of the proteins exclusively identified in two different Dectin1 pull down experiments and not detected in the controls. Galectin-9 is one of the proteins consistently identified in Dectin-1 pull down analysis. The #PSM on the table refers to Peptides Spectral Matches. It is the measure of number of times each peptide is identified.

### Galectin-9 Blockade is Protective Against PDA

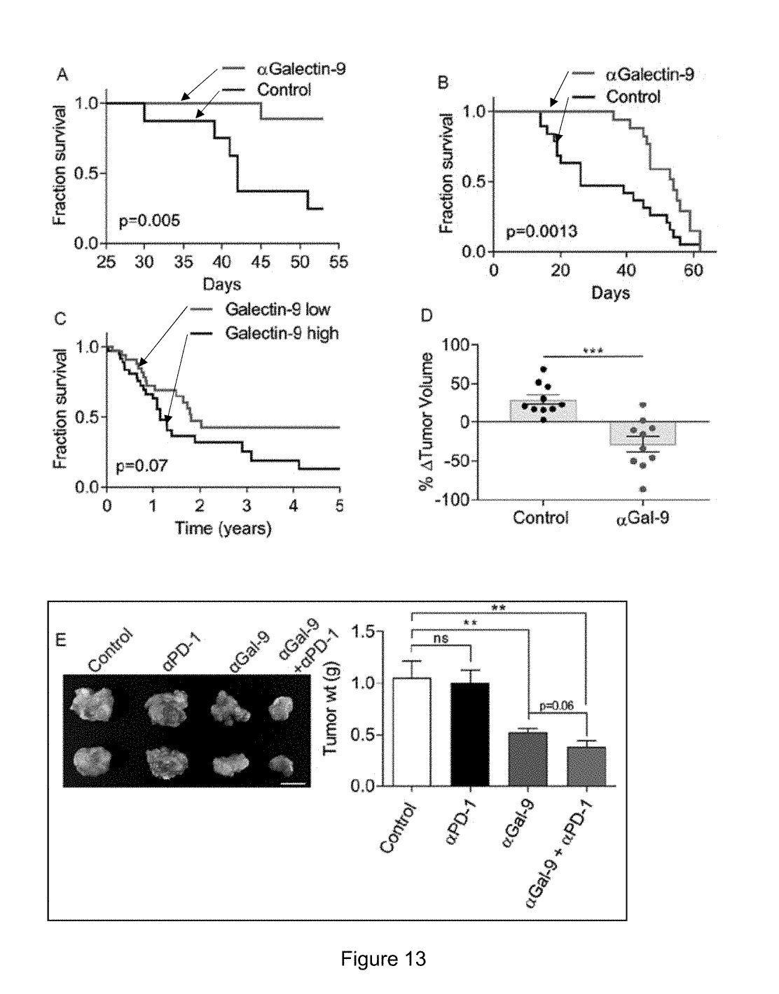

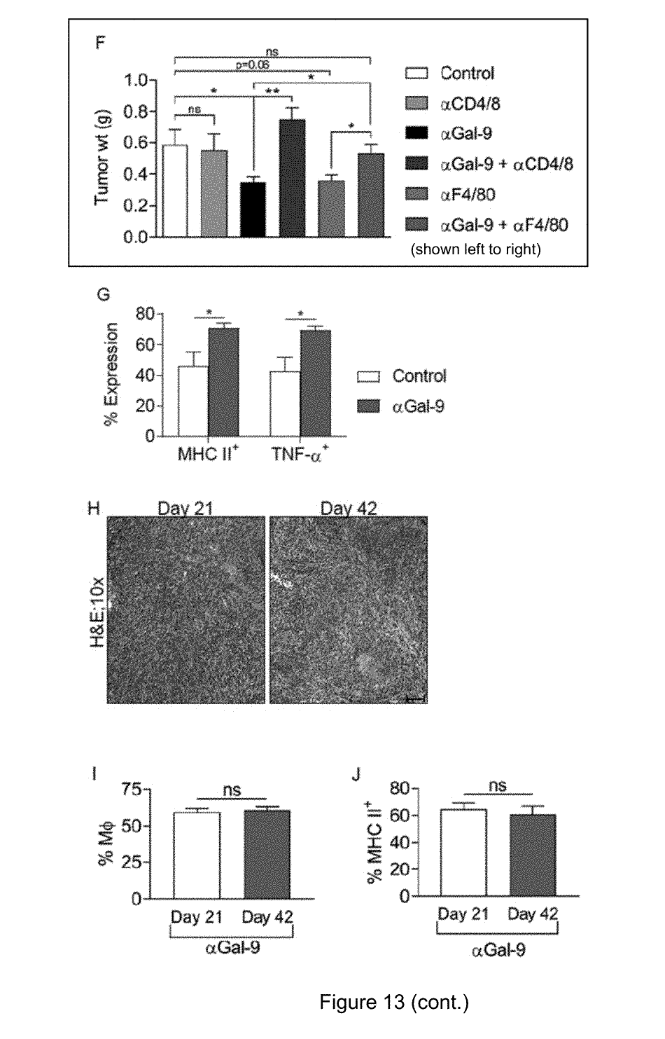

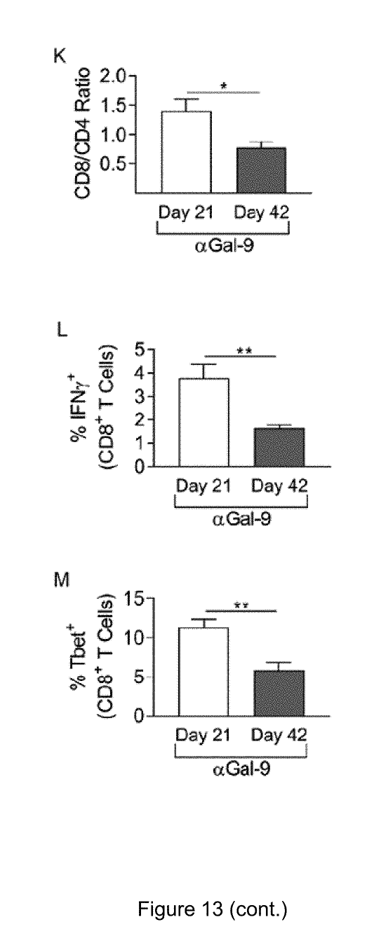

Since it was found that Galectin-9 activates Dectin-1 in PDA, it was postulated that Galectin-9 blockade would protect against tumor progression. It was found that serial treatment with a neutralizing α-Galectin-9 monoclonal antibody (beginning one day prior to tumor implantation) extended survival in mice harboring orthotopic KPC tumors (FIG. 13, Panel A). Additionally, WT mice orthotopically implanted with KPC-derived tumor cells and serially treated with a neutralizing α-Galectin-9 monoclonal antibody or isotype control beginning on day 8 after tumor implantation showed that Galectin-9 blockade also extended survival after monoclonal antibody treatment was initiated in mice harboring established orthotopic KPC tumors (FIG. 13, Panel B). Similarly, human PDA patients with respective high or low tertile levels of Galectin-9 expression were compared using the UCSC RNAseq database. Elevated Galectin-9 expression was associated with a trend toward reduced survival in human PDA (FIG. 13, Panel C). To determine whether Galectin-9 blockade induces tumor regression, mice were implanted subcutaneously with PDA cells and beginning on day 8 after tumor was established, animals were treated with a neutralizing α-Galectin-9 monoclonal antibody or isotype control. On day 14, the percentage increase or regression in tumor size compared with day 8 was determined, showing that Galectin-9 blockade resulted in substantial tumor regression (FIG. 13, Panel D). To determine the effect of the combined blockade of Galectin-9 and PD-1, WT mice were orthotopically implanted with KPC-derived tumor cells and serially treated with a neutralizing α-Galectin-9 mAb alone, a neutralizing αPD1 mAb alone, αGalectin-9+αPD1, or isotype controls. Cohorts of mice were sacrificed on day 21. Akin to Dectin-1 deletion, combined blockade of Galectin-9 and PD-1 trended to offer synergistic protection against orthotopic PDA (FIG. 13, Panel E) and resulted in enhanced T cell activation. Moreover, when WT mice were orthotopically implanted with KPC-derived tumor cells and serially treated with neutralizing αCD4 and αCD8 mAbs, a neutralizing αGalectin-9 mAb alone, αF4/80 alone, αGalectin-9+αCD4/αCD8, αGalectin-9+αF4/80, or respective isotype controls and sacrificed on day 21, T cell deletion abrogated the protective effects of Galectin-9 blockade (FIG. 13, Panel F). By contrast, macrophage depletion was protective against PDA in WT hosts as has been previously reported (Seifert, L. et al., Nature, 2016, 532, 245-249); however, depleting macrophages accelerated tumor growth in the context of Galectin-9 neutralization (FIG. 13, Panel F). Further, similar to Dectin-1 deletion, Galectin-9 neutralization was associated with immunogenic reprogramming of TAMs in PDA (FIG. 13, Panel G). These data suggest that Galectin-9 neutralization leads to macrophage-dependent adaptive anti-tumor immunity. Since Galectin-9-based immunotherapy of orthotopic PDA tumors eventually fails—despite more than doubling median survival—the cellular differentiation and inflammatory infiltrate in early (day 21) versus late (day 42) tumors were compared in mice treated with αGalectin-9. Two cohorts of WT mice were orthotopically implanted with KPC-derived tumor cells at 3 week intervals. Each cohort was serially treated with a neutralizing αGalectin-9 mAb or isotype control beginning on day 8 after tumor implantation. Both cohorts of mice were sacrificed together on the same day which coincided with day 21 or day 42 after tumor implantation, respectively (n=7/group). Tumor differentiation, macrophage infiltration, and polarization were similar in early and late tumors (FIG. 13, Panels H to J). However, CD8⁺ T cell infiltration was markedly diminished in advanced tumors and CD8⁺ T cells expressed lower IFN-γ and T-bet (FIG. 13, Panels K to M).

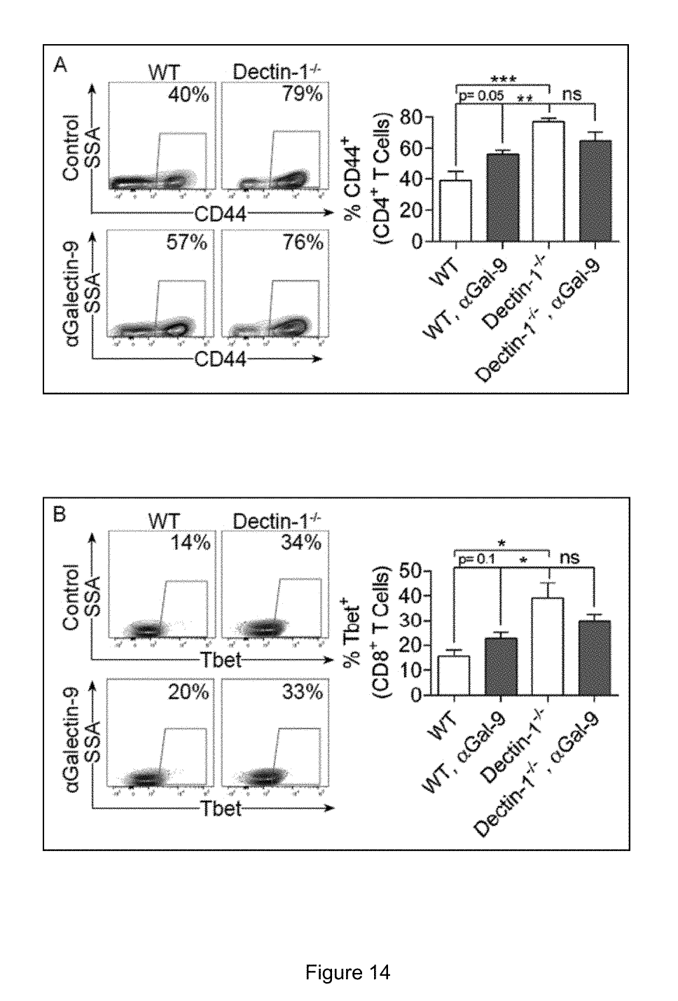

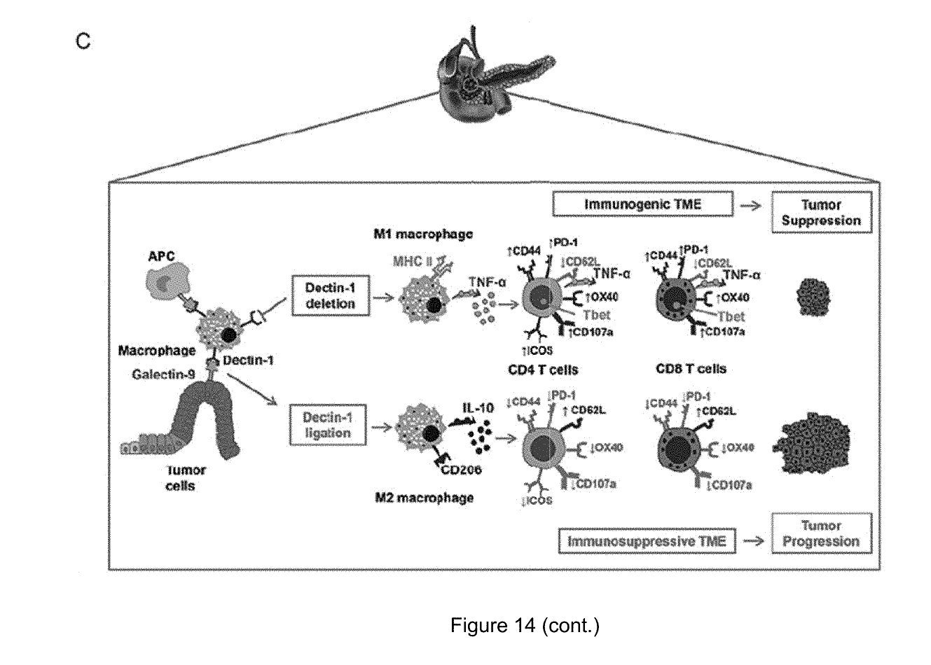

To determine whether the immune-suppressive T cell program associated with Dectin-1 signaling in the PDA TME was contingent on Galectin-9, Galectin-9 was serially blocked in KPC tumor bearing WT and Dectin-1^{-/-} mice with neutralizing αGalectin-9 monoclonal antibody or control. Galectin-9 neutralization enhanced intra-tumoral T cell activation in PDA in WT hosts. However, Galectin-9 neutralization failed to further enhance CD4⁺ or CD8⁺ T cell phenotype in the context of Dectin-1 deletion (FIG. 14, Panels A and B). Collectively, the data suggests that the Dectin-1-Galectin-9 axis μlays a pivotal role in the education of CD4⁺ and CD8⁺ T cells toward immunogenic or tolerogenic phenotypes in PDA, which regulates oncogenic progression (FIG. 14, Panel C).

Dectin-1 is vital in the innate immune defense against fungal pathogens (Vautier et al., Cytokine, 2012, 58, 89-99). Patients with genetic deficiencies in Dectin-1 are at risk for recurrent mucocutaneous fungal infections, such as vulvovaginal candidiasis or onychomycosis (Ferwerda et al., N Engl J Med., 2009, 361, 1760-17670. However, unlike their TLR cousins, a definitive role for Dectin-1 in promoting non-pathogen mediated inflammation or oncogenesis was lacking (Bianchi, J Leukoc Biol., 2007, 81, 1-5). In this study, Dectin-1 was shown to critically regulate macrophage phenotype in PDA, which dictates the immunogenic or tolerogenic properties of peritumoral T cells. Moreover, it is shown that, whereas T cells are dispensable in PDA, as T cell deletion does not influence tumor growth, Dectin-1 deletion renders T cells indispensable to tumor protection. These data suggest that targeting Dectin-1 may be an attractive strategy for PDA immunotherapy in experimental therapeutics.

Beyond elucidating a critical role for Dectin-1 signaling in macrophages in modulating T cell plasticity in the transformed pancreas, one of the most important observations in this study is the discovery that Galectin-9, a lectin with affinity for β-galactosides, is a functional ligand for Dectin-1. This finding could have far-reaching implications to a broader role for Dectin-1 in sterile inflammation and oncogenesis. Galectin-9 has been reputed as an exhaustion ligand for the TIM3 checkpoint receptor on T cells (Zhu et al., Nature Immunol., 2005, 6, 1245-1252). TIM3 is also expressed on macrophages and dendritic cells (Anderson et al, Science, 318, 1141-1143). However, the Galectin-9-TIM3 relationship has recently been called into question (Leitner et al., PLoS Pathogens, 2013, 9, e1003253).

The present data suggest that, akin to Dectin-1, Galectin-9 would be an attractive target for immunotherapy in PDA. In fact, the present data suggest that combination immunotherapy regimens targeting Dectin-1 or Galectin-9 and PD-1 are likely to have synergistic efficacy, with Dectin-1/Galectin-9 blockade enhancing T cell activation and PD-1 blockade preventing exhaustion via checkpoint receptor ligation. Thus, the development of therapeutics targeting Dectin-1 signaling could potentially enable immunotherapeutic options in human PDA.

### Example 2: Expression and Purification of Galectin-9 Targets for Preparing Anti-Galectin-9 Antibodies

Galectin-9, as described above, is a potential therapeutic target. The structure of Galectin-9 is highly conserved between mouse and human (FIG. 15). Cysteine-rich domain 1 (CRD1) shows 70% identity and 83% sequence similarity, while CRD2 has 75% identity and 82% similarity between the two species.

Expression platforms were designed using codon-optimized genes for human and mouse Galectin-9 CRD1 and CRD2 domains. The resulting polynucleotides were cloned into pHBT expression vectors, which are IPTG-inducible, and included an N-terminal 6×HisTag, AviTag, and a TEV cleavage site. The vectors were transformed and expressed in BL21(DE3) E. coli and purified after overnight incubation in 18° C. in 2×TY media and then subjected to Ni-NTA purification. The resulting human and mouse CRD2 fragments was then assayed using an ELISA. High-binding ELISA plates were coated with 0.2 μg/mL of an anti-Galectin-9 (BioXCell) antibody and then the resulting binding complexes were probed with neutravidin-HRP. Both the human and mouse Galectin-9 CRD2 fragments were recognized by the anti-Galectin-9 antibody. FIG. 17.

The purified human and mouse CRD2 fragments are to be used for inducing anti-Galectin-9 antibodies following routine practices.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. A method of treating a solid tumor in a subject, the method comprising administering to a subject in need thereof a therapeutically effective amount of a Dectin-1 antagonist.

2. The method of claim 1, wherein the Dectin-1 antagonist is an inhibitor of Dectin-1.

3. The method of claim 2, wherein the Dectin-1 inhibitor is a small molecule, an antibody, or an interfering RNA (RNAi).

4. The method of claim 1, wherein the Dectin-1 antagonist is an inhibitor of Galectin-9.

5. The method of claim 4, wherein the Galectin-9 inhibitor is a small molecule, an antibody, or an interfering RNA.

6. The method of claim 4, wherein the Galectin-9 inhibitor is an anti-Galectin-9 antibody that binds the CRD1 domain of Galectin-9.

7. The method of claim 4, wherein the Galectin-9 inhibitor is an anti-Galectin-9 antibody that binds the CRD2 domain of Galectin-9.

8. The method of claim 1, wherein the Dectin-1 antagonist is an inhibitor of spleen tyrosine kinase (Syk).

9. The method of claim 8, wherein the inhibitor of Syk blocks phosphorylation of the Syk.

10. The method of claim 9, wherein the inhibitor of Syk is selected from the group consisting of piceatannol, cerdulatinib (P505-15, PRT062607), fostamatinib disodium (R788), nilvadipine, ASN-002, MK-8457, entospletinib, GS-9876, TAK-659, TOP-1288, GSK-2646264, HMPL-523, SKIO-703, TOP-1630, AB-8779, CC-509, CVXL-0074, FF-10102, LAS-189386, PRT-2761, RO-9021, TAS-5567, TOP-1210, CG-103065, DNX-2000, Excellair, HM-029, HMPL-281, Jak3/Syk Dual Inhibitor, PRT-060318, PRT-2607, R-112, R-348, SKI-O-282, SKIO-592, R-333, R-343, C-13, R09021, and R-406.

11. The method of claim 1, further comprising administering to the subject an inhibitor of a checkpoint molecule, an activator of a co-stimulatory receptor, or an inhibitor of an innate immune cell target.

12. The method of claim 11, wherein the checkpoint molecule is selected from the group consisting of PD-1, PD-L1, PD-L2, CTLA-4, LAG3, TIM-3 and A2aR.

13. The method of claim 11, wherein the co-stimulatory receptor is selected from the group consisting of OX40, GITR, CD137, CD40, CD27, and ICOS.

14. The method of claim 11, wherein the innate immune cell target is selected from the group consisting of KIR, NKG2A, CD96, TLR, and IDO.

15. The method of claim 11, wherein the subject is administered an inhibitor of a checkpoint molecule, which is an anti-PD-1 antibody.

16. The method of claim 1, wherein the solid tumor is pancreatic ductal adenocarcinoma (PDA) colorectal cancer (CRC) pancreatic duct adenocarincoma (PDA), colorectal cancer (CRC), melanoma, breast cancer, lung cancer (for example, non-small cell lung cancer, NSCLC, and small cell lung cancer, SCLC), upper and lower gastrointestinal malignancies (including, but not limited to, esophageal, gastric, and hepatobiliary cancer), squamous cell head and neck cancer, genitourinary, and sarcomas.

17. A kit for treating a solid tumor in a subject, the kit comprising:
(a) a first pharmaceutical composition that comprises a Dectin-1 antagonist; and
(b) a second pharmaceutical composition that comprises an inhibitor of a checkpoint molecule, an activator of a co-stimulatory receptor, or an inhibitor of an innate immune cell target.

18. A pharmaceutical composition, comprising (i) a Dectin-1 antagonist, and (ii) an inhibitor of a checkpoint molecule, an activator of a co-stimulatory receptor, or an inhibitor of an innate immune cell target.

19. A method for analyzing a biological sample of a subject, the method comprising:
(a) obtaining a biological sample from a subject suspected of having pancreatic ductal adenocarcinoma (PDA); and
(b) measuring the level of Dectin-1 in the biological sample.

20. The method of claim 19, wherein the biological sample is a blood sample containing cells and the level of Dectin-1 expressed on the cells is measured.

21. The method of claim 19, wherein the biological sample is a tissue sample.

22. The method of claim 19, wherein the measuring step is performed by an immune assay involving an antibody specific to Dectin-1.

23. The method of claim 19, further comprising identifying the subject as having or at risk for PDA based on the level of Dectin-1 determined in step (b), wherein an elevated level of Dectin-1 relative to that of a control subject is indicative of presence or risk of PDA.

24. The method of claim 23, further comprising performing a treatment of PDA to the subject, if the subject is identified as having or at risk for PDA.

25. An isolated antibody, which binds an epitope in CRD1 or CRD2 of a Galectin-9 polypeptide.

26. The isolated antibody of claim 25, wherein the Galectin-9 is a human Galectin-9.

27. The isolated antibody of claim 25, which cross reacts with mouse Galectin-9.

28. The isolated antibody of claim 26, which binds human Galectin-9 more effectively than mouse Galectin-9 by a factor of at least 10-fold.
